SEQUENCE LISTING (5 sequences)

SEQ ID NO: 1 - protein, 775 aa, Artificial Sequence
  Synthetically derived Thermococcus sp. strain 9 degrees N-7 polypeptide
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 2 - protein, 775 aa, Artificial Sequence
  Synthetically derived Thermococcus sp. strain 9 degrees N-7 polypeptide
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIDTLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 3 - DNA, 12 bp, Artificial Sequence
  Synthetic oligonucleotide
atgcatgcatgc

SEQ ID NO: 4 - DNA, 12 bp, Artificial Sequence
  Synthetic oligonucleotide
gcatgcatgcat

SEQ ID NO: 5 - DNA, 18 bp, Artificial Sequence
  Synthetic oligonucleotide
atgcataaaaaagcatgc

(19) United States
(12) Patent Application Publication
(10) Pub. No.: US 2025/0154607 A1
(43) Pub. Date: May 15, 2025

(54) ISOTHERMAL AMPLIFICATION OF PATHOGENS

(71) Applicant: BECTON, DICKINSON AND COMPANY, Franklin Lakes, NJ (US)

(72) Inventors: Honghua ZHANG, San Diego, CA (US); Andrew P. MILLER, San Diego, CA (US); Brion MERMER, San Diego, CA (US); Li-Chun TSAI, San Diego, CA (US)

(21) Appl. No.: 18/548,972

(22) PCT Filed: Mar. 18, 2022
(86) PCT No.: PCT/US2022/021015

§ 371 Date: Sep. 5, 2023

Related U.S. Application Data
(60) Provisional application No. 63/163,399, filed on Mar. 19, 2021.
(60) Provisional application No. 63/307,099, filed on Feb. 5, 2022.

(51) Int. Cl. C12Q 1/689; C12Q 1/6844; C12Q 1/70

(52) U.S. Cl. CPC C12Q 1/689; C12Q 1/6844; C12Q 1/701

(57) ABSTRACT

Disclosed herein include methods, compositions, and kits for use in detecting a target nucleic acid sequence in a sample. The method can comprise the use of a lysis buffer comprising a lytic agent and/or a reducing agent for treating a sample and detecting the presence of a target nucleic acid sequence. In some embodiments, the method comprises contacting a reagent composition comprising amplification agents and one or more protectants (e.g., cyclodextrin compounds) capable of sequestering lytic agents with the treated sample to generate an amplification reaction mixture, for example under isothermal conditions, for detecting.

### RELATED APPLICATIONS

The present application is a U.S. national phase application under 35 U.S.C. § 371 of International Application No. PCT/US2022/021015, filed Mar. 18, 2022 and published as WO2022198086 on Sep. 22, 2022, which claims priority under 35 U.S.C. § 119(e) to U.S. Provisional Application No. 63/163,399, filed Mar. 19, 2021; and U.S. Provisional Application No. 63/307,099, filed Feb. 5, 2022. The entire contents of these applications are hereby expressly incorporated by reference in their entireties.

### REFERENCE TO SEQUENCE LISTING

The present application is being filed along with a Sequence Listing in electronic format. The Sequence Listing is provided as a file entitled Sequence_Listing_68EB-317309-WO, created Mar. 17, 2022, which is 16.0 kilobytes in size. The information in the electronic format of the Sequence Listing is incorporated herein by reference in its entirety.

### BACKGROUND

### Field

The present disclosure relates generally to methods and compositions for amplification (e.g., isothermal amplification) of nucleic acids.

### Description of the Related Art

Nucleic acid-based diagnostics can be useful for rapid detection of infection, disease and/or genetic variations. For example, identification of bacterial or viral nucleic acid in a sample can be useful for diagnosing a particular type of infection. Other examples include identification of single nucleotide polymorphisms for disease management or forensics, and identification of genetic variations indicative of genetically modified food products. Often, nucleic acid-based diagnostic assays require amplification of a specific portion of nucleic acid in a sample. A common technique for nucleic acid amplification is the polymerase chain reaction (PCR). This technique typically requires a cycling of temperatures (i.e., thermocycling) to proceed through the steps of denaturation (e.g., separation of the strands in the double-stranded DNA (dsDNA) complex), annealing of oligonucleotide primers (short strands of complementary DNA sequences), and extension of the primer along a complementary target by a polymerase. Such thermocycling can be a time consuming process that generally requires specialized machinery. Thus, a need exists for quicker nucleic acid amplification methods that can be performed without thermocycling. Such methods may be useful, for example, for on-site testing and point-of-care diagnostics. Moreover, there is a need for such compositions and methods of nucleic acid detection wherein lytic agents employed to lyse biological entities (e.g., viral particles, bacteria) are prevented from inactivating amplification reagents (e.g., polymerases) and wherein the deleterious activity of nucleases (e.g., reduced deoxyribonucleases, reduced ribonucleases) is inhibited at all stages.

### SUMMARY

Disclosed herein include methods for detecting a target nucleic acid sequence in a sample. In some embodiments, the method comprises: (a) contacting a sample comprising biological entities with a lysis buffer to generate a treated sample, wherein the lysis buffer comprises one or more lytic agents capable of lysing biological entities to release sample nucleic acids comprised therein, wherein the sample nucleic acids are suspected of comprising a target nucleic acid sequence. The method can comprise: (b) contacting a reagent composition (e.g., a dried composition) with the treated sample to generate an amplification reaction mixture, wherein the reagent composition comprises one or more protectants and one or more amplification reagents. The method can comprise: (c) amplifying a target nucleic acid sequence in the amplification reaction mixture, thereby generating a nucleic acid amplification product. The method can comprise: (d) detecting the nucleic acid amplification product, wherein the detecting is performed in less than about 20 minutes from the time the reagent composition is contacted with the treated sample.

The lysis buffer and/or reagent composition can comprise one or more reducing agents. In some embodiments, the pH of the lysis buffer is about 1.0 to about 10.0 (e.g., about 2.2). In some embodiments, the lysis buffer comprises one or more of magnesium sulfate, ammonium sulfate, EDTA, and EGTA. In some embodiments, the sample nucleic acids comprise sample ribonucleic acids and/or sample deoxyribonucleic acids. The sample ribonucleic acids can comprise a cellular RNA, a mRNA, a microRNA, a bacterial RNA, a viral RNA, or any combination thereof. The one or more amplification reagents can comprise a reverse transcriptase and/or an enzyme having a hyperthermophile polymerase activity. In some embodiments, the enzyme having a hyperthermophile polymerase activity has a reverse transcriptase activity. In some embodiments, contacting the dried composition with the treated sample comprises dissolving the dried composition in the treated sample. In some embodiments, the reagent composition comprises one or more of a reverse transcriptase, an enzyme having a hyperthermophile polymerase activity, a first primer, a second primer, and a reverse transcription primer. The amplifying can be performed in an isothermal amplification condition. Detecting the nucleic acid amplification product can comprises use of a real-time detection method. The one or more reducing agents can comprise one or more of 2-mercaptoethanol, dithiothreitol (DTT), tris(2-carboxyethyl)phosphine (TCEP), dithioerythritol (DTE), reduced glutathione, cysteamine, tri-n-butylphosphine (TBP), dithioerythriol, tris(3-hydroxypropyl)phosphine (THPP), 2-mercaptoethylamin-HCl, dithiobutylamine (DTBA), cysteine, cysteine-thioglycolate, salts of sulfurous acid, thioglycolic acid and hydroxyethyldisulphide (HED).

The reagent composition can be lyophilized and/or heat dried. The reagent composition can comprise one or more additives (e.g., an amino acid, a polymer, a sugar or sugar alcohol). The sugar or sugar alcohol can comprise sucrose, lactose, trehalose, dextran, erythritol, arabitol, xylitol, sorbitol, mannitol, or any combination thereof. The polymer can comprise polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof.

The molar ratio of the one or more protectants to the one or more amplification reagents can be between about 10:1 to about 1:10 (e.g., about 2:1). The molar ratio of the one or more protectants to the one or more lysis reagents can be between about 10:1 to about 1:10 (e.g., about 2:1). The one or more additives can comprise Tween 20, Triton X-100, Tween 80, a non-ionic detergent (e.g., a non-ionic surfactant), or any combination thereof. In some embodiments, the one or more additives help lyophilization of the reaction compositions and/or the dissolution of dried pellets. The one or more additives can comprise a nonionic detergent at a concentration of about 0.01% in the reagent composition (e.g., a dry composition such as dried pellet). In some embodiments, at a low concentration (such as 0.01%) the presence of the one or more additives (e.g., Tween 80 plus Triton x-100, nonionic detergents) in dried pellets improves APA reaction replicates. In some embodiments, the neutralization of ionic detergent by the one or more protectants (e.g., HP-betaCD) alone is sufficient even in the absence of nonionic detergent in lyophilized pellets. In some embodiments, the one or more protectants comprises a cyclodextrin compound of formula (I):

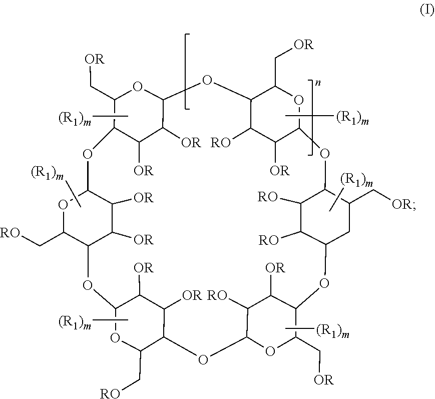

or a salt, ester, solvate or hydrate thereof, wherein,
each R is independently H, alkyl, alkenyl, alkynyl, cycloalkyl, heterocycloalkyl, aryl, or heteroaryl, each of which is optionally substituted; or —C(O)OR^{B}, —OC(O)R^{B}, —C(O)R^{B}, or —C(O)NR^{A}R^{B};
each R₁ is independently H, alkyl, alkenyl, alkynyl, cycloalkyl, aryl, heteroaryl, halogen, hydroxy, amino, —CN, —CF₃, —N₃, —NO₂, —OR^{B}, —SR^{B}, —SOR^{B}, SO₂R^{B}, —N(R^{B})S(O₂)—R^{B}, —N(R^{B}) S(O₂)NR^{A}R^{B}, —NR^{A}R^{B}, —C(O)OR^{B}, —OC(O)R^{B}, —C(O)R^{B}, —C(O)NR^{A}R^{B}, or —N(R^{B})C(O)R^{B}; each of which is optionally substituted;
each R^{A} is independently hydrogen, alkyl, alkenyl, alkynyl, cycloalkyl, heterocycloalkyl, aryl, or heteroaryl, each of which is optionally substituted;
each R^{B} is independently hydrogen, alkyl, alkenyl, alkynyl, cycloalkyl, heterocycloalkyl, aryl, or heteroaryl, each of which is optionally substituted;
n is 1, 2, 3, 4, 5, 6, 7, 8, 9, or 10; and
each m is independently 0, 1, 2, 3, 4, or 5.

In some embodiments, each R is independently H, optionally substituted alkyl, —C(O)OR^{B}, —OC(O)R^{B}, —C(O)R^{B}, or —C(O)NR^{A}R^{B}. In some embodiments, n is 1, 2, or 3. In some embodiments, each R is independently H, methyl, ethyl, propyl, butyl, pentyl, hexyl, heptyl, or octyl; wherein each is straight chain or branched. In some embodiments, the cyclodextrin compound is 2-hydroxypropyl-α-cyclodextrin, 2-hydroxypropyl-β-cyclodextrin (2HPPCD), hydroxypropyl-β-cyclodextrin (HPβCD), methyl-β-cyclodextrin (MPCD), 2-Hydroxypropyl-γ-cyclodextrin, α-cyclodextrin, β-cyclodextrin, or γ-cyclodextrin, or a salt, ester, solvate or hydrate thereof. In some embodiments, 2-hydroxypropyl-α-cyclodextrin is more effective against smaller inhibitors than HPβCD.

The one or more lytic reagents can comprise about 0.001% (w/v) to about 1.0% (w/v) of the treated sample, e.g., about 0.2% (w/v) of the treated sample. In some embodiments, the one or more lytic agents comprise a detergent. In some embodiments, the detergent comprises one or more of a cationic surfactant, an anionic surfactant, a non-ionic surfactant, and an amphoteric surfactant. The anionic surfactant can be any one or more of the anionic surfactants disclosed herein. In some embodiments, the anionic surfactant comprises NH₄⁺, K⁺, Na⁺, or Li⁺ as a counter ion. The cationic surfactant can be any one or more of the cationic surfactants disclosed herein. In some embodiments, the cationic surfactant comprises I⁻, Br⁻, or Cl⁻ as a counter ion.

The sample nucleic acids can comprise a nucleic acid comprising the target nucleic acid sequence. In some embodiments, the target nucleic acid sequence comprises a first strand and a second strand complementary to each other. In some embodiments, amplifying the target nucleic acid sequence comprises: amplifying a target nucleic acid sequence comprising a first strand and a second strand complementary to each other in an isothermal amplification condition, wherein the amplifying comprises contacting a nucleic acid comprising the target nucleic acid sequence with: i) a first primer and a second primer, wherein the first primer is capable of hybridizing to a sequence of the first strand of the target nucleic acid sequence, and the second primer is capable of hybridizing to a sequence of the second strand of the target nucleic acid sequence; and ii) an enzyme having a hyperthermophile polymerase activity, thereby generating a nucleic acid amplification product, wherein the nucleic acid amplification product comprises: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long. In some embodiments, the amplifying does not comprise using any enzyme other than the enzyme having a hyperthermophile polymerase activity, and the amplifying does not comprise heat denaturing and/or enzymatic denaturing the nucleic acid. In some embodiments, the method does not comprise contacting the nucleic acid with a single-stranded DNA binding protein prior to or during step (c). In some embodiments, the nucleic acid is a double-stranded DNA. In some embodiments, the nucleic acid is a product of reverse transcription reaction. In some embodiments, the nucleic acid is a product of reverse transcription reaction generated from sample ribonucleic acids. In some embodiments, step (c) comprises generating the nucleic acid by a reverse transcription reaction. In some embodiments, the method does not comprise thermal or enzymatic denaturation of the sample nucleic acid.

In some embodiments, the sample nucleic acids comprise sample ribonucleic acids, and wherein the method comprises contacting sample ribonucleic acids with a reverse transcriptase and/or a reverse transcription primer to generate a cDNA. In some embodiments, amplifying the target nucleic acid sequence comprises: (c1) contacting sample ribonucleic acids with a reverse transcriptase and/or a reverse transcription primer to generate a cDNA; (c2) contacting the cDNA with an enzyme having a hyperthermophile polymerase activity to generate a double-stranded DNA (dsDNA), wherein the dsDNA comprises a target nucleic acid sequence, and wherein the target nucleic acid sequence comprises a first strand and a second strand complementary to each other; (c3) amplifying the target nucleic acid sequence under an isothermal amplification condition. In some embodiments, the amplifying comprises contacting the dsDNA with: (i) a first primer and a second primer, wherein the first primer is capable of hybridizing to a sequence of the first strand of the target nucleic acid sequence, and the second primer is capable of hybridizing to a sequence of the second strand of the target nucleic acid sequence; and (ii) the enzyme having a hyperthermophile polymerase activity, thereby generating a nucleic acid amplification product, wherein the nucleic acid amplification product comprises: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long. In some embodiments, the method does not comprise using any enzymes other than the reverse transcriptase and/or the enzyme having a hyperthermophile polymerase activity.

In some embodiments, step (d) further comprises determining the amount of the dsDNA and/or nucleic acid that comprises the target nucleic acid sequence in the sample. In some embodiments, the enzyme having a hyperthermophile polymerase activity has an amino acid sequence that is at least about 90% identical or at least about 95% identical to the amino acid sequence of SEQ ID NO: 1 or a functional fragment thereof. In some embodiments, the enzyme having a hyperthermophile polymerase activity is a polymerase comprising the amino acid sequence of SEQ ID NO: 1. In some embodiments, the enzyme having a hyperthermophile polymerase activity has low or no exonuclease activity. Amplifying the target nucleic acid sequence can be performed at a constant temperature of about 55 degrees Celsius (“° C.”) to about 75° C., for example about 65° C.

The first primer, the second primer, or both can be about 8 to 17 bases long. In some embodiments, the first primer, the second primer, or both comprises one or more of DNA bases, modified DNA bases, or a combination thereof. The nucleic acid amplification product can be about 20 to 40 bases long. In some embodiments, the spacer sequence comprises a portion of the target nucleic acid sequence. In some embodiments, the spacer sequence is 1 to 10 bases long.

In some embodiments, the method comprises: contacting the nucleic acid amplification product with a signal-generating oligonucleotide capable of hybridizing to the amplification product, wherein the signal-generating oligonucleotide comprises a fluorophore, a quencher, or both. In some embodiments, detecting the nucleic acid amplification product comprises detecting a fluorescent signal. In some embodiments, the fluorescent signal is from a molecular beacon. In some embodiments, the method is performed in a single reaction vessel. In some embodiments, the sample ribonucleic acids are contacted with the reverse transcriptase and the enzyme having a hyperthermophile polymerase activity simultaneously. In some embodiments, the sample ribonucleic acids are contacted with the reverse transcriptase, the enzyme having a hyperthermophile polymerase activity, and the first and second primers simultaneously. In some embodiments, the sample ribonucleic acids are contacted with the reverse transcriptase, the enzyme having a hyperthermophile polymerase activity, the first primer, the second primer, and the reverse transcription primer simultaneously.

In some embodiments, the one or more lytic agents are capable of denaturing the one or more amplification reagents, and wherein the one or more protectants are capable of preventing the denaturing of the one or more amplification reagents by the one or more lytic agents. In some embodiments, the one or more protectants are capable of sequestering the one or more lytic agents, thereby preventing the denaturing of the one or more amplification reagents by the one or more lytic agents. In some embodiments, greater than about 90% of the one or more lytic agents are sequestered by the one or more protectants. In some embodiments, less than about 10% of the one or more amplification reagents are denatured by the one or more lytic agents.

In some embodiments, the sample comprises a plurality of nucleases (e.g., deoxyribonucleases, ribonucleases). In some embodiments, the one or more lytic agents are capable of denaturing the nucleases (e.g., deoxyribonucleases, ribonucleases) to generate denatured nucleases (e.g., denatured deoxyribonucleases, denatured ribonucleases), wherein the one or more reducing agents are capable of reducing the nucleases to generate reduced nucleases (e.g., reduced deoxyribonucleases, reduced ribonucleases), and wherein the one or more reducing agents and the one or more lytic agents are capable of generating reduced-denatured nucleases (e.g., reduced-denatured deoxyribonucleases, reduced-denatured ribonucleases). In some embodiments, denatured ribonucleases and/or reduced-denatured ribonucleases are capable of renaturing upon passage of time and/or upon reduced physical interaction with the one or more lytic agents, thereby generating renatured ribonucleases. In some embodiments, denatured deoxyribonucleases and/or reduced-denatured deoxyribonucleases are capable of renaturing upon passage of time and/or upon reduced physical interaction with the one or more lytic agents, thereby generating renatured deoxyribonucleases. In some embodiments, the sequestering of the one or more lytic agents reduces the physical interaction of the one or more lytic agents with denatured deoxyribonucleases, reduced-denatured deoxyribonucleases, denatured ribonucleases and/or reduced-denatured ribonucleases. In some embodiments, denatured deoxyribonucleases, reduced deoxyribonucleases, reduced-denatured deoxyribonucleases, denatured ribonucleases, reduced ribonucleases, and/or reduced-denatured ribonucleases are enzymatically inactive, and wherein renatured ribonucleases and/or renatured deoxyribonucleases are enzymatically active. In some embodiments, reduced-denatured ribonucleases renature at least about 1.1-fold more slowly than denatured ribonucleases. In some embodiments, reduced-denatured deoxyribonucleases renature at least about 1.1-fold more slowly than denatured deoxyribonucleases. In some embodiments, about 1.1-fold more sample ribonucleic acids are employed by the reverse transcriptase as template to generate cDNA as compared to a comparable method wherein the lysis buffer does not comprise one or more reducing agents. In some embodiments, about 1.1-fold more sample deoxyribonucleic acids are employed by an enzyme having a hyperthermophile polymerase activity as template to generate a nucleic acid amplification product as compared to a comparable method wherein the lysis buffer does not comprise one or more reducing agents. In some embodiments, the amplification reaction mixture comprises an at least 1.1-fold lower nuclease (e.g., deoxyribonuclease, ribonuclease) activity ten minutes after step (b) and/or step (c) as compared to a comparable method wherein the lysis buffer does not comprise one or more reducing agents.

The biological entities can comprise one or more of prokaryotic cells, eukaryotic cells, viral particles, exosomes, protoplasts, and microvesicles. In some embodiments, the biological entities comprise a virus, a bacteria, a fungi, a protozoa, portions thereof, or any combination thereof. In some embodiments, the target nucleic acid sequence is a nucleic acid sequence of a virus, bacteria, fungi, or protozoa. In some embodiments, the sample nucleic acids are derived from a virus, bacteria, fungi, or protozoa. The virus can comprise one or more of SARS-CoV-2, Human Immunodeficiency Virus Type 1 (HIV-1), Human T-Cell Lymphotrophic Virus Type 1 (HTLV-1), Hepatitis B Virus (HBV), Hepatitis C Virus (HCV), Herpes Simplex, Herpesvirus 6, Herpesvirus 7, Epstein-Barr Virus, Respiratory Syncytial Virus (RSV), Cytomegalo-virus, Varicella-Zoster Virus, JC Virus, Parvovirus B19, Influenza A, Influenza B, Influenza C, Rotavirus, Human Adenovirus, Rubella Virus, Human Enteroviruses, genital Human Papillomavirus (HPV), and Hantavirus. In some embodiments, the bacteria comprises one or more of Mycobacteria tuberculosis, Rickettsia rickettsii, Ehrlichia chaffeensis, Borrelia burgdorferi, Yersinia pestis, Treponema pallidum, Chlamydia trachomatis, Chlamydia pneumoniae, Mycoplasma pneumoniae, Mycoplasma sp., Legionella pneumophila, Legionella dumoffii, Mycoplasma fermentans, Ehrlichia sp., Haemophilus influenzae, Neisseria meningitidis, Neisseria gonorrhoeae, Streptococcus pneumonia, S. agalactiae, and Listeria monocytogenes. In some embodiments, the fungi comprises one or more of Cryptococcus neoformans, Pneumocystis carinii, Histoplasma capsulatum, Blastomyces dermatitidis, Coccidioides immitis, and Trichophyton rubrum. In some embodiments, the protozoa comprises one or more of Trypanosoma cruzi, Leishmania sp., Plasmodium, Entamoeba histolytica, Babesia microti, Giardia lamblia, Cyclospora sp., and Eimeria sp.. In some embodiments, the amplifying step comprises multiplex amplification of two or more target nucleic acid sequences, and where the detecting step comprises multiplex detection of two more nucleic acid amplification products derived from said two or more target nucleic acid sequences. In some embodiments, the two or more target nucleic acid sequences are specific to two or more different organisms. In some embodiments, the two or more different organisms comprise one or more of Chlamydia trachomatis, Neisseria gonorrhoeae, SARS-CoV-2, Influenza A, Influenza B, and/or Influenza C.

In some embodiments, the amplifying comprises and/or does not comprise, one or more of the following: loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA). In some embodiments, the amplifying does not comprise LAMP. In some embodiments, the method does not comprise one or more of the following: (i) dilution of the treated sample; (ii) dilution of the amplification reaction mixture; (iii) heat denaturation of the treated sample; (iv) sonication of the treated sample; (v) sonication of the amplification reaction mixture; (vi) the addition of ribonuclease inhibitors and/or deoxyribonuclease inhibitors to the treated sample; (vii) the addition of ribonuclease inhibitors and/or deoxyribonuclease inhibitors to the amplification reaction mixture; (viii) purification of the sample; (ix) purification of the sample nucleic acids; (x) purification of the nucleic acid amplification product; (xi) removal of the one or more lytic agents from the treated sample or the amplification reaction mixture; (xii) heating denaturing and/or enzymatic denaturing of the sample nucleic acids prior to and/or during amplification; and (xiii) the addition of ribonuclease H to the treated sample or amplification reaction mixture. In some embodiments, the sample is held at an amplification temperature (e.g., 67° C.). In some embodiments, the sample is held at temperature between room temperature and reaction temperature for 1-2 minutes before amplification reaction to facilitate reverse transcription reaction for RNA samples

Disclosed herein include kits for detecting a target nucleic acid sequence in a sample. In some embodiments, the kit comprises: (a) a lysis buffer comprising one or more lytic agents, wherein the one or more lytic agents are capable of lysing biological entities to release sample nucleic acids comprised therein, wherein the sample nucleic acids are suspected of comprising a target nucleic acid sequence; and (b) a reagent composition (e.g., a dried composition) comprising one or more protectants and one or more amplification reagents, wherein the one or more amplification reagents comprise one or more components for amplifying a target nucleic acid sequence under isothermal amplification conditions. In some embodiments, said components comprise: (i) a first primer and a second primer, wherein the first primer is capable of hybridizing to a sequence of a first strand of the target nucleic acid sequence, and the second primer is capable of hybridizing to a sequence of a second strand of the target nucleic acid sequence; and (ii) an enzyme having a hyperthermophile polymerase activity capable of generating a nucleic acid amplification product. In some embodiments, the lysis buffer and/or reagent composition comprises one or more reducing agents. The one or more reducing agents can comprise one or more of 2-mercaptoethanol, DTT, TCEP, DTE, reduced glutathione, cysteamine, TBP, dithioerythriol, THPP, 2-mercaptoethylamin-HCl, DTBA, cysteine, cysteine-thioglycolate, salts of sulfurous acid, thioglycolic acid and HED.

The kit can comprise: at least one component providing real-time detection activity for a nucleic acid amplification product. In some embodiments, the real-time detection activity is provided by a molecular beacon. In some embodiments, the reagent composition (e.g., the dried composition) comprises a reverse transcriptase and/or a reverse transcription primer. The enzyme having a hyperthermophile polymerase activity can have an amino acid sequence that is at least about 90% identical or at least about 95% identical to the amino acid sequence of SEQ ID NO: 1 or a functional fragment thereof. In some embodiments, the enzyme having a hyperthermophile polymerase activity is a polymerase comprising the amino acid sequence of SEQ ID NO: 1. In some embodiments, the nucleic acid amplification product is about 20 to 40 bases long, and wherein the nucleic acid amplification product comprises: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

The molar ratio of the one or more protectants to the one or more amplification reagents can be between about 10:1 to about 1:10, e.g., about 2:1. In some embodiments, the one or more additives comprise Tween 20, Triton X-100, Tween 80, a non-ionic detergent (e.g., a non-ionic surfactant), or any combination thereof. In some embodiments, the one or more protectants comprises a cyclodextrin compound of formula (I). In some embodiments, the one or more protectants are capable of sequestering the one or more lytic agents, thereby preventing the denaturing of the one or more amplification reagents by the one or more lytic agents. In some embodiments, the one or more lytic reagents comprise about 0.001% (w/v) to about 1.0% (w/v) of the treated sample, for example about 0.2% (w/v) of the treated sample. In some embodiments, the one or more lytic agents comprise a detergent. The detergent can comprise one or more of a cationic surfactant, an anionic surfactant, a non-ionic surfactant, and an amphoteric surfactant

### BRIEF DESCRIPTION OF THE DRAWINGS

FIGS. 1A-lB show a non-limiting exemplary schematic of an isothermal amplification reaction provided herein.

FIGS. 2A-2B depict data related to the effect of anionic detergent SDS and HP-βCD on Neisseria gonorrhea DNA amplification. Assays with no protective agent (FIG. 2A) or with 4% HP-βCD (FIG. 2B) are shown.

FIGS. 3A-3B depict data related to the effect of cationic detergent CTAB on Chlamydia trachomatis DNA Amplification.

FIG. 4 depicts data related to lysis of Chlamydia trachomatis.

FIGS. 5A-5B depict data related to the effect of hydroxypropyl-β-cyclodextrin (HPβCD) protection on Flu B virus detection.

FIG. 6 depicts data related to an order-of-addition assay.

FIG. 7 depicts data related to the effect of DTT on the irreversibility of RNase A inactivation in the presence of CTAB at various temperatures.

FIG. 8 depicts data related to effect of DTT and HP-βCD on RNase A inactivation.

FIG. 9 depicts data related to requirements for irreversible RNase inactivation.

FIG. 10A depicts data related to protection of RNA from nasal swab ribonucleases. FIG. 10B depicts data related to an assay calibration to assess degree of protection.

FIGS. 11A-11B depict data related to RNase A activity at 580 without βCD (FIG. 11A) and with βCD (FIG. 11B).

FIGS. 12A-12B depict data related to RNase A inactivation in an influenza B assay with DTT in ELB (Elution/Lysis Buffer).

FIG. 13 depicts data related to the rapid irreversibility of nasal ribonuclease inactivation.

FIGS. 14A-14B depict data related to direct lysis for isothermal influenza B virus detection in the presence and absence of clinical matrix.

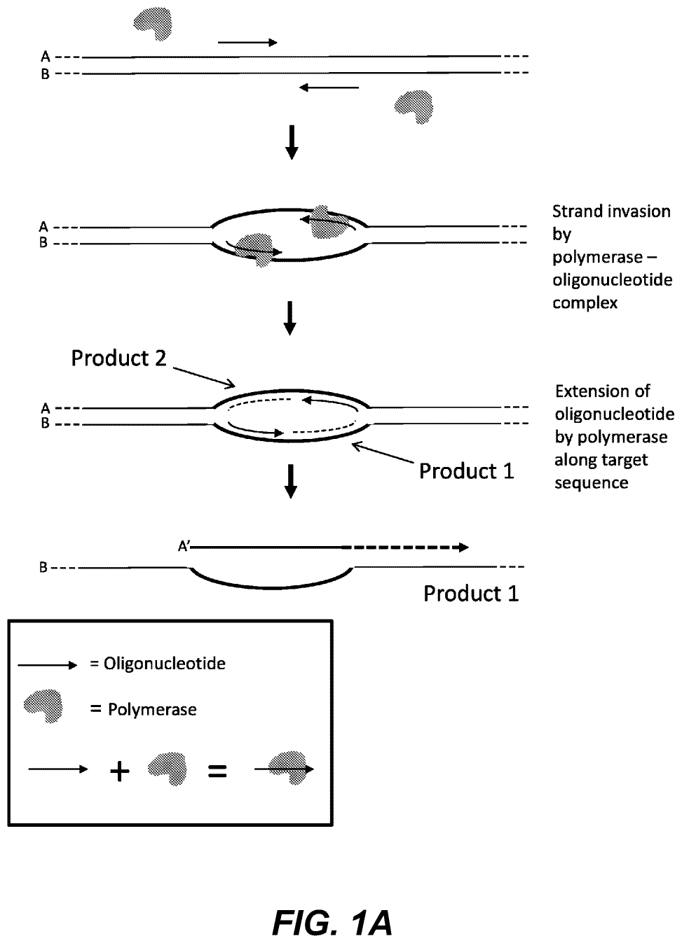

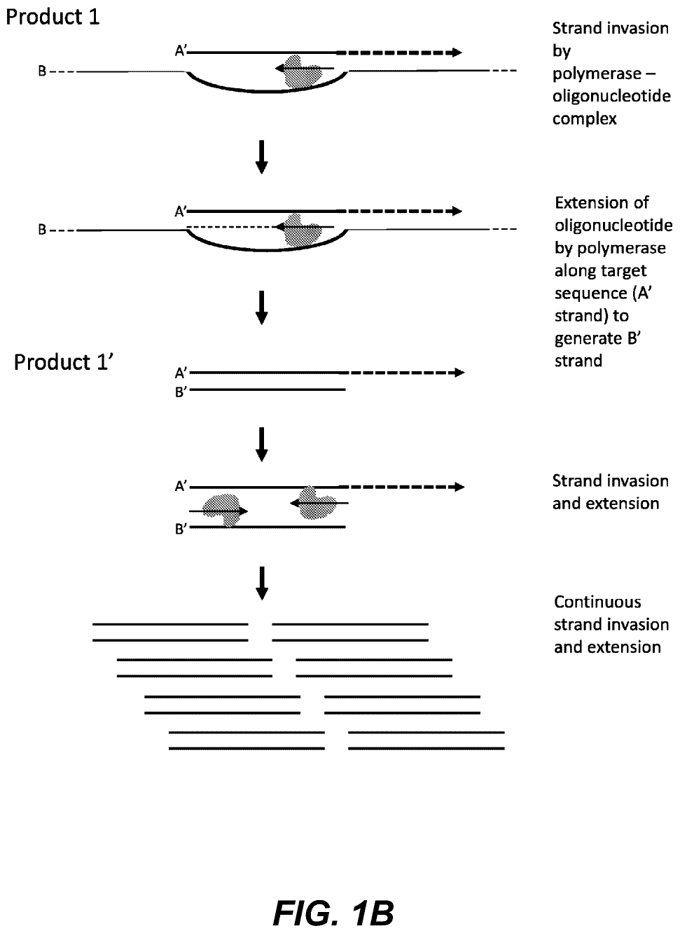

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

Disclosed herein include methods for detecting a target nucleic acid sequence in a sample. In some embodiments, the method comprises: (a) contacting a sample comprising biological entities with a lysis buffer to generate a treated sample, wherein the lysis buffer comprises one or more lytic agents, wherein the one or more lytic agents are capable of lysing biological entities to release sample nucleic acids comprised therein, wherein the sample nucleic acids are suspected of comprising a target nucleic acid sequence. The method can comprise: (b) contacting a reagent composition (e.g., a dried composition) with the treated sample to generate an amplification reaction mixture, wherein the reagent composition comprises one or more protectants and one or more amplification reagents. The method can comprise: (c) amplifying a target nucleic acid sequence in the amplification reaction mixture, thereby generating a nucleic acid amplification product. The method can comprise: (d) detecting the nucleic acid amplification product, wherein the detecting is performed in less than about 20 minutes, about 15 minutes, about 10 minutes, about 5 minutes, or about 2.5 minutes, from the time the reagent composition is contacted with the treated sample.

Disclosed herein include kits for detecting a target nucleic acid sequence in a sample. In some embodiments, the kit comprises: (a) a lysis buffer comprising one or more lytic agents, wherein the one or more lytic agents are capable of lysing biological entities to release sample nucleic acids comprised therein, wherein the sample nucleic acids are suspected of comprising a target nucleic acid sequence; and (b) a reagent composition (e.g., a dried composition) comprising one or more protectants and one or more amplification reagents, wherein the one or more amplification reagents comprise one or more components for amplifying a target nucleic acid sequence under isothermal amplification conditions. In some embodiments, said components comprise: (i) a first primer and a second primer, wherein the first primer is capable of hybridizing to a sequence of a first strand of the target nucleic acid sequence, and the second primer is capable of hybridizing to a sequence of a second strand of the target nucleic acid sequence; and (ii) an enzyme having a hyperthermophile polymerase activity capable of generating a nucleic acid amplification product. The lysis buffer and/or reagent composition can comprise one or more reducing agents (including but not limited one or more of 2-mercaptoethanol, DTT, TCEP, DTE, reduced glutathione, cysteamine, TBP, dithioerythriol, THPP, 2-mercaptoethylamin-HCl, DTBA, cysteine, cysteine-thioglycolate, salts of sulfurous acid, thioglycolic acid and HED).

Provided herein are methods and compositions for amplifying nucleic acid. Traditional nucleic acid amplification methods typically require a thermocycling process, nucleic acid denaturation, proteins (e.g., enzymes) that promote strand unwinding, strand separation and/or strand exchange (e.g., helicases, recombinases), and/or endonuclease agents (e.g., restriction enzymes, nicking enzymes), and often require a minimum reaction time of 20 to 30 minutes. The nucleic acid amplification methods provided herein can be performed without thermocycling, without thermal denaturation and/or enzymatic denaturation of sample nucleic acids, without added proteins (e.g., enzymes) to promote strand unwinding, strand separation and/or strand exchange, without endonuclease agents, and within a reaction time of about 10-15 minutes.

### Methods of Nucleic Acid Amplification

Provided herein include, methods and compositions for amplification (e.g., isothermal amplification) of nucleic acids derived from a sample, for example in an ionic detergent-based lysis solution. In some embodiments, the methods include lysis of biological entities (e.g., pathogens) in a sample followed directly by isothermal amplification and real-time detection of released RNA target sequences. In some embodiments, these methods advantageously demonstrate inactivation of nucleases (e.g., deoxyribonucleases, ribonucleases) and prevention of DNA and/or RNA degradation in clinical and physiological samples. Methods and compositions are also provided herein for direct lysis of viral and bacterial pathogens in clinical samples prior to isothermal nucleic acid amplification and real-time detection without any purification or separation steps. Disclosed herein, in some embodiments, is a two-step method comprised of a first step chemical lysis of viral particles or bacterial cells in clinical samples containing nucleases (e.g., deoxyribonucleases, ribonucleases) using lytic agents that inactivate nucleases followed by a second step wherein a chemical agent can prevent reactivation of the nucleases under subsequent reactions conditions that favor enzyme activity. In some embodiments, the two-step method provided herein does not comprise sample purification, and/or nucleic acid isolation/purification. Also provided herein are one-step methods comprising simultaneous chemical lysis and inactivation of nucleases (e.g., deoxyribonucleases, ribonucleases) when both lytic agents and reducing agents are present in lysis buffer. In some embodiments, the methods provided herein are two-step methods.

Provided herein include methods and compositions for direct pathogen lysis in clinical samples to enable isothermal amplification using an archaeal polymerase and real-time detection of nucleic acids. Rapid point of care (POC) diagnostics can be developed that do not require sample purification. Viral particles, bacterial cells, or other pathogens still need be lysed so that their DNA and RNA can be released from the cell and can be available for an amplification reaction. Conventional chemical lysis methods (e.g., employing strong bases, ionic detergents, and chaotropic agents) are incompatible with enzyme function, as these agents will also inactivate any DNA polymerase or other enzymes. Therefore, there is significant value in an effective chemical lysis method that is compatible with enzyme function and does not require any nucleic acid purification steps to remove the lysis reagents.

In some embodiments, a clinical sample containing pathogen is added to a lysis buffer containing a detergent (e.g., a potent ionic detergent) to lyse the biological entity (e.g., target pathogen) to release nucleic acid. The lysis solution can be transferred directly into a reaction containing lyophilized amplification reagents and a protectant against the ionic detergent that effectively neutralizes the chemical detergents used in the lysis buffer, thereby enabling the reaction to proceed without intermediate purification or separation steps. The methods and compositions provided herein advantageously enable a direct, rapid target amplification reaction with patient samples (as used herein, the term “direct” can mean no nucleic acid purification or dilution of the sample following its delivery into an ionic-detergent based lysis solution designed to liberate the target organisms' protective coating). This solution can then be used to dissolve the dried reaction reagents. Since the lytic ionic detergents are incompatible with the amplification chemistry, the dried reagents to accomplish that chemistry can include a cyclodextrin which sequesters the ionic detergent, preventing what would otherwise be a deleterious interaction with enzymes in the reaction chemistry. Reagents and drying conditions provided herein can be such that, upon delivery of the lysis solution to the dried reagents, the cyclodextrin dissolves more rapidly than the enzymes and/or functions rapidly enough to protect the enzymes, since even very brief exposure of the enzymes to the ionic detergent would be adequate to inactivate the enzymes. While nucleic acid amplification methods such as PCR and RT-PCR assays have a theoretical detection limit of one genomic copy per reaction, the presence of inhibitors in the clinical samples can limit the functioning of enzymes for nucleic acid amplification. Thus, for molecular detection of pathogens in clinical samples, currently available methods often involve purification or separation steps for nucleic acids (DNA or RNA) amplification. Ionic detergents are commonly used in bacterial cell lysis, and RNA or DNA extraction. Due to the strong denaturing impact and inhibition of ionic detergents on amplification methods such as PCR using Taq polymerase, the ionic detergent cannot be used for pathogen lysis without a separation or purification steps. In contrast to other methods, the disclosed compositions and methods provide direct lysis of pathogens in clinical samples using ionic detergents and allows isothermal amplification and real-time detection of the released nucleic acids without purification or separation steps, thereby reducing the assay time and complexity, as well as the risk of contamination.

The compositions and methods disclosed herein can overcome a series of obstacles encountered while developing a system for rapid, point-of-care, isothermal target amplification/detection of RNA viruses like influenza or coronavirus in patient respiratory samples. As the most effective viral lysis method, a potent ionic detergent, such as sodium dodecyl sulfate (SDS), was selected for proof-of-principle experiments. SDS can inhibit a variety of detrimental enzymes normally present in the sample. In a direct test, the same lysis agent that can inhibit harmful enzymes can also inhibit the enzymes needed to accomplish the amplification/detection reaction. To overcome that obstacle, the SDS was sequestered by inclusion of a cyclodextrin compound in the lyophilized reaction mixture which contained enzymes for subsequent amplification of the released ribonucleic acids. It was then found, however, that this sequestration can also allow rapid reactivation of detrimental ribonuclease enzyme activity from the sample. To overcome that reactivation obstacle, reducing agents can be included during the lysis step. Thus, detrimental ribonuclease activity can be rapidly and irreversibly inactivated, resulting in intact ribonucleic acids ready for amplification without a need for separation or purification. More precisely, patient respiratory samples containing RNA viruses can inevitably contain ubiquitous ribonucleases. During the intended procedure for rapid molecular Archaeal Polymerase Amplification (APA) testing for these viral RNAs, there can be two steps during which the target RNA would be susceptible to destruction by these ribonucleases. The first lytic step occurs after delivery of the sample to a lysis buffer containing lytic agents, which is designed to release viral RNA from its protective coatings, to produce a lysate. The released RNA would thus no longer be protected from ribonuclease activity. The second reaction step can begin with direct dissolution by this lysate of dried APA reagents, with continued susceptibility of the RNA to sample ribonucleases. Risk of RNA destruction can persist until the included enzyme reverse transcriptase had completed a DNA copy of the RNA target segment as a prelude to amplification and detection of that DNA copy by the APA process, or any isothermal process. Ionic detergents, such as SDS and/or cetyltrimethylammonium bromide (CTAB), can be employed as lytic agents in the methods and compositions provided herein, and can allow efficient lysis and also inhibit ribonucleases by denaturation, thus protecting the target RNA during the lysis step. However the subsequent direct, rapid molecular testing reaction, initiated by dissolution of a dried reaction mix directly by the lysed sample without separation or nucleic acid purification, can require rapid removal of the SDS (and/or CTAB), since it would also denature the enzymes reverse transcriptase and polymerase needed for the reaction. However, removal of some agents used to denature ribonuclease could thus enable its reactivation, i.e. restoration of its enzymatic activity. As disclosed herein, rapid removal of SDS (and/or CTAB) can be enabled by inclusion of a cyclodextrin in the dried reaction mix. This rapid removal may, in fact, also accelerate refolding of some denatured proteins. As disclosed herein, it was indeed shown that ribonuclease reactivation was very rapid in the presence of cyclodextrin, leaving the target RNA unprotected. The addition of reducing agents during ribonuclease denaturation by some denaturants can delay reactivation following relatively slow removal of the denaturant and the reducing agents, usually hours. As described herein, the addition of reducing agents during the lysis step, in combination with the SDS (and/or CTAB), instantly rendered the ribonuclease inactivation sufficiently irreversible to enable enzyme-mediated molecular detection, despite the likely acceleration of ribonuclease refolding due to rapid removal of the SDS (and/or CTAB) by cyclodextrins after the brief lysis step. The reducing agents can be compatible with enzymatic detection so need not be removed in some embodiments.

The methods and compositions provided herein provide various unexpected advantages over currently available nucleic acid detection methods and compositions, for example, those described in U.S. Pat. Nos. 5,705,345, 5,422,241, 6,777,210, and 6,204,375; U.S. Patent Application Publication Nos. 20170152546 and 20140322761, Anfinsen, Christian B., et al. (Proc Natd Acad Sci USA 47.9 (1961): 1309), Anand (IJC 2013), and Yamaguchi, Hiroshi (Biomolecules 4.1 (2014): 235-251).

For example, currently available methods use cyclodextrin to neutralize an ionic detergent-containing lysis solution to enable subsequent amplification in wet reactions (not with dried reagents). Those available methods require addition of cyclodextrin to the detergent lysate, an extra step inconsistent with point-of-care molecular diagnostics as disclosed herein. In some embodiments of the methods provided herein, lysate is added to dried reagents containing CD, under conditions where CD can dissolve before enzymes. In contrast, currently available methods, after neutralization of detergent at 0.5% SDS, use dilution into the amplification reaction mixture for final 0.05% SDS (i.e., diluted into amplification reaction, inconsistent with point-of-care). Provided herein, the neutralization and amplification can occur in the same solution, at 0.2% SDS. The currently available methods also include nucleic acids being immobilized on a solid support, such as an FTE paper, and the lysis reagent is embedded onto the solid matrix. However, even though the sample type can extend to human blood, which is easy to lyse, such methods do not provide efficient lysis nor amplification for hard-to-lyse pathogens. As such, these currently available methods have limited use for clinical samples and are also substantially different from those disclosed herein. Moreover, the currently available methods fail to achieve equivalent performance without SDS or SDS plus cyclodextrins for RNA targets with reverse transcriptase.

The compositions and methods currently available (e.g., for ribonuclease inactivation) are shown to be inadequate in the context of the applications contemplated herein. For example, those currently available compositions and methods only shows efficacy post-inactivation, not during (e.g., in this context during the reaction step and the lytic step, respectively). Moreover, currently available methods require at least 4 minutes at 60 0° C. for irreversibility while the compositions and methods provided herein can permit virtually immediate irreversible inactivation at 68° C., enabling a more rapid lysis step, an essential element in a point-of-care application. For example, Myhrvold et al (Science. 2018 April 27; 360(6387): 444-448) discloses the inactivation of RNases with multi-step, cumbersome and unacceptably slow prior art methods, and does not disclose the compositions (e.g., lysis buffers, protectants) and methods provided herein. Additionally, currently available methods employ a non-calibrated method of unknown sensitivity to assess residual ribonuclease activity while provided herein the assessment of ribonuclease activity after inactivation used the intended molecular diagnostic test as the efficacy test. Furthermore, currently available methods can only achieve irreversible ribonuclease inactivation at a maximum 200 ng/ml while the compositions and methods provided herein showed utility with 10 μg/ml to accommodate potentially larger amounts in patient samples. Moreover, currently available methods attempt to show efficacy after ribonuclease inactivation, not during. Additionally, currently available methods are designed for analytical applications wherein pure RNA and ribonuclease are intentionally mixed and incubated prior to addition of the denaturants, and with an additional extraction, separation, or purification step involving treatment with reagents which would purify undigested RNA and ribonuclease by precipitation, with unexplored effect on recovery of ribonuclease activity, a process incompatible with a rapid point-of-care application. Unlike currently available methods showing delayed reactivation of ribonuclease after lengthy denaturation in the presence of the denaturant urea and reducing agents and then relatively slow removal of both, provided herein are conditions that achieve a similar but rapid, immediate impact on adequately preventing ribonuclease reactivation when rapid renaturation was achieved by rapid removal of only the denaturing ionic detergent. Moreover, the current hypothesis of the impact of cyclodextrins on refolding of various proteins but not necessarily their reactivation, has not been applied to ribonucleases. Additionally, it is currently unknown if the time scales involved in renaturation and reactivation following cyclodextrin treatment of ribonucleases (e.g., RNase A) follows the same unfolding and refolding pathways as model proteins studied. Furthermore, currently available methods contemplate the inclusion of ribonuclease inhibitors to enable the process to proceed to a next enzymatic step after removal of the ionic detergent (rather than inclusion of reducing agents during inactivation).

Although currently available methods contemplated effective reverse-transcription of RNA in samples containing ribonucleases, without nucleic acid purification, directly after ribonuclease inactivation with reducing agents, none were so designed to enable inclusion of harsh chemical viral lysis reagents, such as anionic detergent SDS, or cationic detergent CTAB, as required for a rapid molecular diagnostic as described herein. Unexpectedly, and as shown herein, there was essentially instantaneous inactivation of ribonuclease by denaturant and reducing agent, allowing direct subsequent reverse transcription, as rapidly as needed for the applications contemplated herein.

Unexpectedly, and as described herein, operational ribonuclease irreversibility under conditions required for a rapid molecular test required ionic detergent in addition to reducing agent when followed by selective rapid removal of the detergent. Bender et al (J Mol Diagn. 2020 Aug;22(8):1030-1040) discloses the inactivation of endogenous ribonucleases using methods comprising long incubations, proteinase K, and/or elevated incubation temperatures, which are advantageously avoided by the methods and compositions provided herein. The methods and compositions provided herein overcome various problems in the currently available methods to achieve real direct, rapid molecular RNA testing in patient samples. Specifically, the lytic agents provided herein can be an ionic detergent, such as SDS or CTAB, which also inactivates nucleases (e.g., deoxyribonucleases, ribonucleases) during the lytic step. The inclusion of a second component, a reducing agent, such as dithiothreitol (DTT) or cysteine, can ensure that during the reaction step, upon removal of the detergent by cyclodextrin, that ribonuclease reactivation is prevented, at least long enough until a DNA copy of the viral RNA has been produced. As disclosed herein, operational irreversibility of ribonuclease inactivation can be achieved virtually immediately during lysis at 68° C. With only ionic detergent, but without reducing agent, although ribonuclease activity was prevented during lysis (in the presence of the detergent), after treatment with cyclodextrin to remove detergent, ribonuclease activity was fully and rapidly recovered and interfered with viral RNA detection. As disclosed herein, without ionic detergent but only reducing agent ribonuclease activity rendered RNA undetectable, presumably due to ribonuclease activity both during lysis and during the reaction. Thus, as described herein, simultaneous treatment with ionic detergent and reducing agent was required for successful detection under these conditions.

In some embodiments, the disclosed methods and compositions enable isothermal amplification and real-time detection of nucleic acids without a need for sample separation or purification for point-of-care molecular diagnostics for direct pathogen lysis in clinical samples. There is provided, in some embodiments, a lysis buffer containing a potent ionic detergent that can be used to lyse pathogens in clinical samples. The amplification reagents can comprise a protectant against the lysis reagent, and can be dried (e.g., lyophilized, heat dried) and used for the amplification of the released nucleic acids in point-of-care settings.

The compositions and methods provided herein enable point-of-care molecular diagnostics for RNA/RNA viruses in clinical samples that can rapidly and irreversibly inactivate nucleases (e.g., deoxyribonucleases, ribonucleases) without a need for separation, purification or extraction for the amplification of the deoxyribonucleic acids and/or ribonucleic acids. The methods and compositions provided herein are compatible with a variety of lysis buffers and lytic agents, including, but not limited to, anionic detergents, alkyl Sulfates, alkyl Sulfonates, bile salts, and cationic detergents including alkyltrimethylammonium surfactants with various alkyl chain lengths.

Ionic detergents are potent chemicals for cell lysis. Ionics detergent such as SDS solubilize the phospholipid and protein components of the cell membrane, leading to cell lysis and release of the cell contents. SDS can have an inhibitory effect at a concentration greater than 0.01% (w/v) for PCR with Taq polymerase, as well as amplification with archaeal polymerases. Cyclodextrins (CDs) are cyclic oligosaccharides which resemble truncated cones with hydrophobic inner cavity and hydrophilic outer surface. Natural cyclodextrins include a, 3 and γ-CD. These CDs can have limited solubility, while chemical modified CD derivatives such as hydroxypropyl 0-CD can improve solubility up to 50% in aqueous media. j-CD forms a strong inclusion complex (more so than α-CD and T-CD) with ionic detergents such as sodium dodecyl sulfate (SDS) in a predominately 1:1 stoichiometry. An ionic detergent can be used in lysis buffer for cell lysis/sample preparation. As described herein, cyclodextrins can be incorporated in a dried (e.g., lyophilized, heat dried) pellet containing enzyme and other assay components as a protectant against the harmful effect from an ionic detergent. After the lysis step, sample solution can be mixed with a dried (e.g., lyophilized, heat dried) pellet containing cyclodextrin to facilitate CD and SDS inclusion complexation.

Rapid, direct molecular testing for RNA viruses in respiratory samples requires an initial lysis step to release viral RNA from its protective coating comprised of carbohydrate, fats, and proteins. The lysis must sufficiently disrupt, e.g. denature, protein structure, in an irreversible manner, to render the RNA intact and accessible to the subsequent enzyme-mediated amplification step. Direct molecular testing means that this same harsh lysis solution must then dissolve reaction reagents/enzymes and thus be compatible with the enzyme-mediated amplification step. Since enzymes are proteins, the protein-denaturing elements which accomplished lysis must be removed, and done so rapidly in this context. To render the lysis solution compatible with the amplification step, there are provided, in some embodiments, dried amplification reagents comprising a cyclodextrin compound which sequesters the ionic detergent immediately upon dissolution of the dried reagents with the lysis solution, before the ionic detergent has denatured the amplification enzymes. In some embodiments, the dissolution of the dried reagents is designed so that this order of dissolution obtains. In some embodiments, the dissolution of the dried reagents is designed so that this order of events occurs. Respiratory samples containing RNA viruses also contain potentially interfering ribonuclease enzymes. Depending upon the method used to achieve lysis, release of viral RNA could render it susceptible to destruction by these ribonucleases. An advantage of lysis via ionic detergents is that ribonucleases, as proteins, are also denatured and therefore inactive during the lysis step. Ribonucleases, however, demonstrate the highly unusual property, compared with most other proteins, that denaturation may be reversible, e.g. renaturation (with restoration of enzymatic activity) occurs upon removal of the denaturing agent. Thus, upon removal of the ionic detergent by the cyclodextrins provided herein after dissolving the dried reagents, sample ribonucleases can become reactivated and able to interfere with RNA reverse transcription. It had not been previously shown that sequestration of ionic detergents after treatment accelerated renaturation of ribonucleases. The addition of reducing agents during denaturation of ribonucleases, for example, with denaturants like urea rather than detergents, can delay renaturation following relatively slow removal of both. This may be the case because the reducing agents caused modification of ribonuclease sulfhydryl amino acid residues, preventing their essential role in guiding ribonuclease refolding and restoration of enzymatic activity. As described herein, reducing agents did prevent, or sufficiently delay, subsequent reactivation of ribonucleases which would interfere with RNA detection upon sequestration of ionic detergent denaturants with cyclodextrin. This disclosure provides the first demonstration of the effectiveness of the disclosed combination of treatments, e.g. ionic detergent with reducing agent, such that ribonuclease inactivation was sufficiently rapid and persistent following selective, rapid removal of the ionic detergent with cyclodextrin. All of these properties can be essential in a rapid, direct molecular test for RNA viruses in respiratory samples.

The methods and compositions provided herein can be applied to other amplification methods for sample preparation without purification or separation, for example, PCR, RT-PCR, or other isothermal amplification methods. The methods and compositions provided herein can also be applied to genome sequencing methods or any nucleic acids (DNA or RNA) amplification or detection methods that require a sample preparation step. The methods and compositions provided herein can also find use in genotyping, diagnostics and forensics. The disclosed methods and compositions are not limited to isothermal amplification methods, but rather can be applied to other amplification/detection methods, for example, RT-PCR, WGS sequencing, and can also be applied to RNA purification/extraction without separation.

As described herein, it was unexpectedly found that the kinetics of hydroxypropyl β-CD was sufficiently rapid at a molar ratio of 2:1 of hydroxypropyl β-CD and SDS. Inactivation of ribonucleases by denaturation in the presence of reducing agents and reactivation during or after removal of the inactivating agents can occur. However, after denaturation with ionic detergents in the presence of reducing agents, the kinetics of reactivation upon rapid detergent removal by cyclodextrins had not previously been demonstrated. Unexpectedly, and as described herein, it was found that the kinetics of reactivation under these conditions, in the absence of reducing agents, were sufficiently rapid that they could interfere with subsequent production of a DNA copy of RNA mediated by the enzyme reverse transcriptase. This was the case despite the fact that only a very short, about 30 nucleotide segment of RNA was reverse-transcribed in a matter of seconds. Although not previously demonstrated in the art, as described herein it was unexpectedly found that inclusion of reducing agents prior to cyclodextrin treatment delayed reactivation of ribonucleases sufficiently to enable completion of the reverse transcription and subsequent amplification and detection of the DNA copy. Advantageously, the effectively irreversible inactivation of ribonucleases thus observed during the practice of the methods, kits and compositions disclosed herein was virtually instantaneous, unlike currently available methods based upon heat, and that currently available methods, such as those d described above, under various different conditions, were not sufficiently irreversible to be effective for the rapid diagnostic applications under the conditions described herein.

Disclosed herein include methods for detecting a target nucleic acid sequence in a sample. In some embodiments, the method comprises: (a) contacting a sample comprising biological entities with a lysis buffer to generate a treated sample, wherein the lysis buffer comprises one or more lytic agents, wherein the one or more lytic agents are capable of lysing biological entities to release sample nucleic acids comprised therein, wherein the sample nucleic acids are suspected of comprising a target nucleic acid sequence. The method can comprise: (b) contacting a reagent composition (e.g., a dried composition) with the treated sample to generate an amplification reaction mixture, wherein the reagent composition comprises one or more protectants and one or more amplification reagents. The method can comprise: (c) amplifying a target nucleic acid sequence in the amplification reaction mixture, thereby generating a nucleic acid amplification product. The method can comprise: (d) detecting the nucleic acid amplification product, wherein the detecting is performed in less than about 20 minutes (e.g., about 20, 19, 18, 17, 16, 15, 14, 13, 12, 11, 10, 9, 8, 7, 6, 5, 4, 3, 2, or 1 minute(s), or a number or a range between any two of these values) from the time the reagent composition is contacted with the treated sample. In some embodiments, steps (b) and (c) are performed concurrently (e.g., the amplification begins once the contacting of the dried composition and the treated sample has occurred). In some embodiments, the reagent composition comprises two or more reagent compositions (comprising the same or different components). In some embodiments, the lysis buffer comprises two or more lysis buffers (comprising the same or different components).

The sample nucleic acids can comprise sample ribonucleic acids and/or sample deoxyribonucleic acids. The sample ribonucleic acids can comprise a cellular RNA, a mRNA, a microRNA, a bacterial RNA, a viral RNA, or any combination thereof. The one or more amplification reagents can comprise a reverse transcriptase and/or an enzyme having a hyperthermophile polymerase activity. In some embodiments, the enzyme having a hyperthermophile polymerase activity has a reverse transcriptase activity. Contacting the reagent composition (e.g., dried composition) with the treated sample can comprise dissolving the reagent composition in the treated sample. The reagent composition can comprise one or more of a reverse transcriptase, an enzyme having a hyperthermophile polymerase activity, a first primer, a second primer, and a reverse transcription primer. The amplifying can be performed in an isothermal amplification condition. Detecting the nucleic acid amplification product can comprise use of a real-time detection method.

The one or more lytic reagents can comprise about 0.001% (w/v) to about 1.0% (w/v) of the treated sample, e.g., about 0.2% (w/v) of the treated sample. The sample nucleic acids can comprise a nucleic acid comprising the target nucleic acid sequence. The target nucleic acid sequence can comprise a first strand and a second strand complementary to each other.

Amplifying the target nucleic acid sequence can comprise: amplifying a target nucleic acid sequence comprising a first strand and a second strand complementary to each other in an isothermal amplification condition, wherein the amplifying comprises contacting a nucleic acid comprising the target nucleic acid sequence with: i) a first primer and a second primer, wherein the first primer is capable of hybridizing to a sequence of the first strand of the target nucleic acid sequence, and the second primer is capable of hybridizing to a sequence of the second strand of the target nucleic acid sequence; and ii) an enzyme having a hyperthermophile polymerase activity, thereby generating a nucleic acid amplification product, wherein the nucleic acid amplification product comprises: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

In some embodiments, the amplifying does not comprise using any enzyme other than the enzyme having a hyperthermophile polymerase activity, and the amplifying does not comprise denaturing the nucleic acid. In some embodiments, the method does not comprise contacting the nucleic acid with a single-stranded DNA binding protein prior to or during step (c). In some embodiments, the method does not comprise thermal or enzymatic denaturation of the sample nucleic acid.

The nucleic acid can be a double-stranded DNA. The nucleic acid can be a product of reverse transcription reaction. The nucleic acid can be a product of reverse transcription reaction generated from sample ribonucleic acids. Step (c) can comprise generating the nucleic acid by a reverse transcription reaction.

The sample nucleic acids can comprise sample ribonucleic acids. The method can comprise contacting sample ribonucleic acids with a reverse transcriptase and/or a reverse transcription primer to generate a cDNA. Amplifying the target nucleic acid sequence can comprise: (c1) contacting sample ribonucleic acids with a reverse transcriptase and/or a reverse transcription primer to generate a cDNA; (c2) contacting the cDNA with an enzyme having a hyperthermophile polymerase activity to generate a dsDNA, wherein the dsDNA comprises a target nucleic acid sequence, and wherein the target nucleic acid sequence comprises a first strand and a second strand complementary to each other; (c3) amplifying the target nucleic acid sequence under an isothermal amplification condition, wherein the amplifying comprises contacting the dsDNA with: (i) a first primer and a second primer, wherein the first primer is capable of hybridizing to a sequence of the first strand of the target nucleic acid sequence, and the second primer is capable of hybridizing to a sequence of the second strand of the target nucleic acid sequence; and (ii) the enzyme having a hyperthermophile polymerase activity, thereby generating a nucleic acid amplification product, wherein the nucleic acid amplification product comprises: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

In some embodiments, the method does not comprise using any enzymes other than the reverse transcriptase and/or the enzyme having a hyperthermophile polymerase activity. Step (d) further can comprise determining the amount of the dsDNA and/or nucleic acid that comprises the target nucleic acid sequence in the sample. In some embodiments, the enzyme having a hyperthermophile polymerase activity has an amino acid sequence that is at least about 90% or at least about 95% identical to the amino acid sequence of SEQ ID NO: 1 or a functional fragment thereof. The enzyme having a hyperthermophile polymerase activity can be a polymerase comprising the amino acid sequence of SEQ ID NO: 1. In some embodiments, the enzyme having a hyperthermophile polymerase activity has low or no exonuclease activity.

Amplifying the target nucleic acid sequence can be performed at a constant temperature of about 55° C. to about 75° C., for example about 65° C. The first primer, the second primer, or both can be about 8 to 17 bases long. The first primer, the second primer, or both can comprise one or more of DNA bases, modified DNA bases, or a combination thereof. The nucleic acid amplification product can be about 20 to 40 bases long. The spacer sequence can comprise a portion of the target nucleic acid sequence. The spacer sequence can be 1 to 10 bases long.

In some embodiments, the method comprises: contacting the nucleic acid amplification product with a signal-generating oligonucleotide capable of hybridizing to the amplification product. The signal-generating oligonucleotide can comprise a fluorophore, a quencher, or both. Detecting the nucleic acid amplification product can comprise detecting a fluorescent signal. The fluorescent signal can be from a molecular beacon. The method can be performed in a single reaction vessel. The sample ribonucleic acids can be contacted with the reverse transcriptase and the enzyme having a hyperthermophile polymerase activity simultaneously. The sample ribonucleic acids can be contacted with the reverse transcriptase, the enzyme having a hyperthermophile polymerase activity, and the first and second primers simultaneously. In some embodiments, the sample ribonucleic acids are contacted with the reverse transcriptase, the enzyme having a hyperthermophile polymerase activity, the first primer, the second primer, and the reverse transcription primer simultaneously. Reverse transcription of the sample ribonucleic acids can occur by the addition of a reverse transcription primer. In some embodiments, the reverse transcription primer is an oligo(dT) primer, random hexanucleotide primer, or a target-specific oligonucleotide primer. In some embodiments, oligo(dT) primers are 12-18 nucleotides in length and bind to the endogenous poly(A)+ tail at the 3′ end of mRNA. Random hexanucleotide primers can bind to sample ribonucleic acids at a variety of complementary sites. Target-specific oligonucleotide primers typically selectively prime the sample ribonucleic acids of interest. The first primer and/or second primer can be a reverse transcription primer.

The amplifying step can comprise multiplex amplification of two or more target nucleic acid sequences. The detecting step can comprise multiplex detection of two more nucleic acid amplification products derived from said two or more target nucleic acid sequences. The two or more target nucleic acid sequences can be specific to two or more different organisms, including but not limited to, one or more of Chlamydia trachomatis, Neisseria gonorrhoeae, SARS-CoV-2, Influenza A, Influenza B, and/or Influenza C.

In some embodiments, the amplification comprises one or more of the following amplification methods: APA, LAMP, HDA, RPA, SDA, NASBA, TMA, NEAR, RCA, MDA, RAM, cHDA, SPIA, SMART, 3SR, GEAR and IMDA. In some embodiments, the amplifying does not comprise one or more of the following: LAMP, HDA, RPA, SDA, NASBA, TMA, NEAR, RCA, MDA, RAM, cHDA, SPIA, SMART, 3SR, GEAR and IMDA. In some embodiments, the amplifying does not comprise LAMP.

In some embodiments, the method does not comprise one or more, or any, of the following: (i) dilution of the treated sample; (ii) dilution of the amplification reaction mixture; (iii) heat denaturation of the treated sample; (iv) sonication of the treated sample; (v) sonication of the amplification reaction mixture; (vi) the addition of nuclease inhibitors to the treated sample; (vii) the addition of nuclease inhibitors to the amplification reaction mixture; (viii) purification of the sample; (ix) purification of the sample nucleic acids; (x) purification of the nucleic acid amplification product; (xi) removal of the one or more lytic agents from the treated sample or the amplification reaction mixture; (xii) heating denaturing and/or enzymatic denaturing of the sample nucleic acids prior to and/or during amplification; and (xiii) the addition of ribonuclease H to the treated sample or amplification reaction mixture. In some embodiments, the sample is held at an amplification temperature (e.g., 67° C.). Step (a), step (b), step (c), and/or step (d) can be performed for a period of about 20, 15, 10, 5, 2.5, or 1 minute(s). In some embodiments, step (a), step (b), step (c), and/or step (d) comprises sonication, osmotic shock, chemical treatment, heating, or any combination thereof.

The one or more lytic agents can be capable of denaturing the one or more amplification reagents. The one or more protectants can be capable of preventing the denaturing of the one or more amplification reagents by the one or more lytic agents. The one or more protectants can be capable of sequestering (e.g., complexing) the one or more lytic agents, thereby preventing the denaturing of the one or more amplification reagents by the one or more lytic agents. In some embodiments, at least about 5% (e.g., at least about 5%, 10%, 15%, 20%, 25%, 30%, 35%, 40%, 45%, 50%, 55%, 60%, 65%, 70%, 75%, 80%, 85%, 90%, 95%, or 100%, or a number or a range between any two of these values) of the one or more lytic agents in the amplification reaction mixture are sequestered by the one or more protectants. In some embodiments, less than about 40%, 30%, 20%, 10%, 5%, 3%, 1%, 0.1%, 0%, or a number or a range between any of these values, of the one or more amplification reagents are denatured by the one or more lytic agents.

The sample can comprise a plurality of nucleases (e.g., deoxyribonucleases, ribonucleases). The one or more lytic agents can be capable of denaturing the nucleases to generate denatured nucleases (e.g., denatured deoxyribonucleases, denatured ribonucleases). The one or more reducing agents can be capable of reducing the ribonucleases to generate reduced nucleases (e.g., reduced deoxyribonucleases, reduced ribonucleases). The one or more reducing agents and the one or more lytic agents can be capable of generating reduced-denatured nucleases (e.g., reduced-denatured deoxyribonucleases, reduced-denatured ribonucleases). Denatured ribonucleases and/or reduced-denatured ribonucleases can be capable of renaturing upon passage of time and/or upon reduced physical interaction with the one or more lytic agents, thereby generating renatured ribonucleases. In some embodiments, denatured deoxyribonucleases and/or reduced-denatured deoxyribonucleases are capable of renaturing upon passage of time and/or upon reduced physical interaction with the one or more lytic agents, thereby generating renatured deoxyribonucleases. In some embodiments, the sequestering of the one or more lytic agents reduces the physical interaction of the one or more lytic agents with denatured deoxyribonucleases, reduced-denatured deoxyribonucleases, denatured ribonucleases and/or reduced-denatured ribonucleases. Denatured deoxyribonucleases, reduced deoxyribonucleases, reduced-denatured deoxyribonucleases, denatured ribonucleases, reduced ribonucleases, and/or reduced-denatured ribonucleases can be enzymatically inactive. The renatured nucleases can be enzymatically active. Renaturing can comprise refolding. “Refolding,” as used herein, shall be given its ordinary meaning, and shall also describe any process, reaction or method which transforms disulfide bond containing polypeptides from an improperly folded or unfolded state to a native or properly folded conformation with respect to disulfide bonds.

Reduced-denatured nucleases (e.g., reduced-denatured deoxyribonucleases, reduced-denatured ribonucleases) can renature at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) more slowly than denatured nucleases (e.g., denatured deoxyribonucleases, denatured ribonucleases). At least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) more sample ribonucleic acids and/or sample deoxyribonucleic acids can be employed by the reverse transcriptase and/or the enzyme having a hyperthermophile polymerase activity as template to generate cDNA and/or a nucleic acid amplification product, respectively, as compared to a comparable method wherein the lysis buffer does not comprise one or more reducing agents.

The amplification reaction mixture can comprise an at least 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold, or a number or a range between any of these values) lower nuclease (e.g., deoxyribonuclease, ribonuclease) activity 1, 2, 3, 4, 5, 6, 7, 8, 9, 10, 11, 12, 13, 14, 15, 16, 17, 18, 19, and/or 20 minutes after step (b) and/or step (c) of the methods provided herein as compared to a comparable method wherein the lysis buffer does not comprise one or more reducing agents. In some embodiments, at least about 5% (e.g., at least about 5%, 10%, 15%, 20%, 25%, 30%, 35%, 40%, 45%, 50%, 55%, 60%, 65%, 70%, 75%, 80%, 85%, 90%, 95%, or 100%, or a number or a range between any two of these values) of the nucleases in the sample, the treated sample, and/or the amplification reaction mixture are enzymatically inactive.

In some embodiments, cDNA and/or nucleic acid amplification product is generated from at least about 5% (e.g., at least about 5%, 10%, 15%, 20%, 25%, 30%, 35%, 40%, 45%, 50%, 55%, 60%, 65%, 70%, 75%, 80%, 85%, 90%, 95%, or 100%, or a number or a range between any two of these values) of the sample ribonucleic acids and/or sample deoxyribonucleic acids, respectively. In some embodiments, less than about 30% (e.g., 30%, 20%, 10%, 5%, 3%, 1%, 0.1%, or a number or a range between any of these values) of sample ribonucleic acids and/or sample deoxyribonucleic acids are degraded by nucleases prior to being used as template in a reverse transcription reaction and/or amplification reaction, respectively.

The term “isothermal amplification reaction” shall be given its ordinary meaning and shall also include reactions wherein the temperature does not significantly change during the reaction. In some embodiments, the temperature of the isothermal amplification reaction does not deviate by more than 10° C., for example by not more than 5° C. or by not more than 2° C. during the main enzymatic reaction step where amplification takes place. Depending on the method of isothermal amplification of nucleic acids, different enzymes can be used for amplification. Isothermal amplification compositions and methods are described in PCT Application published as WO2017176404, the content of which is incorporated herein by reference in its entirety.

In some embodiments, the methods and components described herein comprise a storage-stable lysis buffer. In some embodiments, the lysis buffer is resistant to the formation of a precipitate for a period of time under a storage condition (e.g., storage-stable lysis buffer). Compositions, kits, and methods wherein lysis buffers resist precipitation are described in the U.S. Provisional Patent Application No. 63/307,092 entitled “NON-OPAQUE LYTIC BUFFER COMPOSITION FORMULATIONS” and filed on Feb. 5, 2022, the content of which is incorporated herein by reference in its entirety.

Some embodiments of the methods and compositions provided herein do not comprise agents and/or conditions that denature nucleic acids (e.g., promote strand separation and/or promote unwinding) other than acids and/or low pH conditions. Compositions, kits, and methods for nucleic acid detection wherein nucleic acid strands are dissociated under low pH conditions (e.g., via contact with an acidic lysis buffer) to facilitate subsequent rapid amplification and detection are described in the U.S. Provisional Patent Application No. 63/307,085 entitled “METHOD FOR SEPARATING GENOMIC DNA FOR AMPLIFICATION OF SHORT NUCLEIC ACID TARGETS” and filed on Feb. 5, 2022, the content of which is incorporated herein by reference in its entirety.

Provided herein include methods for amplifying nucleic acid. In some embodiments, the method comprises contacting sample nucleic acid under isothermal amplification conditions with components comprising a) at least one oligonucleotide, which at least one oligonucleotide comprises a polynucleotide complementary to a target sequence in the sample nucleic acid, and b) at least one component providing hyperthermophile polymerase activity, thereby generating a nucleic acid amplification product. In some embodiments, the method comprises contacting sample nucleic acid under isothermal amplification conditions with a) non-enzymatic components comprising at least one oligonucleotide, which at least one oligonucleotide comprises a polynucleotide complementary to a target sequence in the sample nucleic acid, and b) an enzymatic component consisting of a hyperthermophile polymerase or a polymerase comprising an amino acid sequence that is at least about 90% identical to a hyperthermophile polymerase, thereby generating a nucleic acid amplification product. In some embodiments, the method comprises contacting sample nucleic acid under isothermal amplification conditions with a) non-enzymatic components comprising at least one oligonucleotide, which at least one oligonucleotide comprises a polynucleotide complementary to a target sequence in the sample nucleic acid, and b) enzymatic activity consisting of i) hyperthermophile polymerase activity and, optionally, ii) reverse transcriptase activity, thereby generating a nucleic acid amplification product.

Also provided herein include methods for determining the presence, absence or amount of a target sequence in sample nucleic acid, comprising a) amplifying a target sequence in the sample nucleic acid, where the target sequence comprises a first strand and a second strand, the first strand and second strand are complementary to each other, and the amplifying comprises contacting sample nucleic acid under helicase-free and/or recombinase-free isothermal amplification conditions with i) a first oligonucleotide and a second oligonucleotide, where the first oligonucleotide comprises, or consists of, a first polynucleotide continuously complementary to a sequence in the first strand, and the second oligonucleotide comprises, or consists of, a second polynucleotide continuously complementary to a sequence in the second strand; and ii) at least one component providing a hyperthermophile polymerase activity, thereby generating a nucleic acid amplification product, where the nucleic acid amplification product comprises, or consists of, 1) a first nucleotide sequence that is continuously complementary to or substantially identical to the first polynucleotide of the first oligonucleotide, 2) a second nucleotide sequence that is continuously complementary to or substantially identical to the second polynucleotide of the second oligonucleotide, and 3) a spacer sequence comprising 1 to 10 bases, and the spacer sequence is flanked by the first nucleotide sequence and the second nucleotide sequence; and b) detecting the nucleic acid amplification product, where detecting the nucleic acid amplification product comprises use of a real-time detection method and is performed in 10 minutes or less from the time the sample nucleic acid is contacted with (a)(i) and (a)(ii), whereby the presence, absence or amount of a target sequence in sample nucleic acid is determined.

Nucleic acid, subjects, samples and nucleic acid processing

Provided herein are methods and compositions for amplifying nucleic acid. The terms “nucleic acid” and “nucleic acid molecule” may be used interchangeably herein. The terms refer to nucleic acids of any composition, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid can be, or can be from, a plasmid, phage, autonomously replicating sequence (ARS), centromere, artificial chromosome, chromosome, or other nucleic acid able to replicate or be replicated in vitro or in a host cell, a cell, a cell nucleus, a mitochondria, or cytoplasm of a cell. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid may be used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame, “forward” strand or “reverse” strand) and double-stranded polynucleotides. The term “gene” means the segment of DNA involved in producing a polypeptide chain; and generally includes regions preceding and following the coding region (leader and trailer) involved in the transcription/translation of the gene product and the regulation of the transcription/translation, as well as intervening sequences (introns) between individual coding segments (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size may be expressed as a number of bases.

In some embodiments of the methods provided herein, one or more nucleic acid targets are amplified. Target nucleic acids may be referred to as target sequences, target polynucleotides, and/or target polynucleotide sequences, and may include double-stranded and single-stranded nucleic acid molecules. Target nucleic acid may be, for example, DNA or RNA. Where the target nucleic acid is an RNA molecule, the molecule may be, for example, double-stranded, single-stranded, or the RNA molecule may comprise a target sequence that is single-stranded. Where the target nucleic acid is double stranded, the target nucleic acid generally includes a first strand and a second strand. A first strand and a second strand may be referred to as a forward strand and a reverse strand and generally are complementary to each other. Where the target nucleic acid is single stranded, a complementary strand may be generated, for example by polymerization and/or reverse transcription, rendering the target nucleic acid double stranded and having a first/forward strand and a second/reverse strand.

A target sequence may refer to either the sense or antisense strand of a nucleic acid sequence, and also may refer to sequences as they exist on target nucleic acids, amplified copies, or amplification products, of the original target sequence. A target sequence can be a subsequence within a larger polynucleotide. For example, a target sequence can be a short sequence (e.g., 20 to 50 bases) within a nucleic acid fragment, a chromosome, a plasmid, that is targeted for amplification. In some embodiments, a target sequence may refer to a sequence in a target nucleic acid that is complementary to an oligonucleotide (e.g., primer) used for amplifying a nucleic acid. Thus, a target sequence may refer to the entire sequence targeted for amplification or may refer to a subsequence in the target nucleic acid where an oligonucleotide binds. An amplification product may be a larger molecule that comprises the target sequence, as well as at least one other sequence, or other nucleotides. The amplification product can be about the same length as the target sequence, for example exactly the same length as the target sequence. The amplification product can comprise, or consist of, the target sequence.

The length of the target sequence, and/or the guanosine cytosine (GC) concentration (percent), may depend, in part, on the temperature at which an amplification reaction is run, and this temperature may depend, in part, on the stability of the polymerase(s) used in the reaction. Sample assays may be performed to determine an appropriate target sequence length and GC concentration for a set of reaction conditions. For example, where a polymerase is stable up to 60° C. to 65° C., then the target sequence may be, for example, from 19 to 50 nucleotides in length, or for example, from about 40 to 50, 20 to 45, 20 to 40, or 20 to 30 nucleotides in length. GC concentration under these conditions may be, for example, less than 60%, less than 55%, less than 50%, or less than 45%.

Target nucleic acid can include, for example, genomic nucleic acid, plasmid nucleic acid, mitochondrial nucleic acid, cellular nucleic acid, extracellular nucleic acid, bacterial nucleic acid and viral nucleic acid. In some embodiments, target nucleic acid may include genomic DNA, chromosomal DNA, plasmid DNA, mitochondrial DNA, a gene, any type of cellular RNA, messenger RNA, bacterial RNA, viral RNA or a synthetic oligonucleotide. Genomic nucleic acid can include any nucleic acid from any genome, for example, animal, plant, insect, viral and bacterial genomes (e.g., genomes present in spores). In some embodiments, genomic target nucleic acid is within a particular genomic locus or a plurality of genomic loci. A genomic locus can include any or a combination of open reading frame DNA, non-transcribed DNA, intronic sequences, extronic sequences, promoter sequences, enhancer sequences, flanking sequences, or any sequences considered associated with a given genomic locus.

The target sequence can comprise one or more repetitive elements (e.g., multiple repeat sequences, inverted repeat sequences, palindromic sequences, tandem repeats, microsatellites, minisatellites, and the like). In some embodiments, a target sequence is present within a sample nucleic acid (e.g., within a nucleic acid fragment, a chromosome, a genome, a plasmid) as a repetitive element (e.g., a multiple repeat sequence, an inverted repeat sequence, a palindromic sequence, a tandem repeat, a microsatellite repeat, a minisatellite repeat and the like). For example, a target sequence may occur multiple times as a repetitive element and one, some, or all occurrences of the target sequence within a repetitive element may be amplified (e.g., using a single pair of primers) using methods described herein. In some embodiments, a target sequence is present within a sample nucleic acid (e.g., within a nucleic acid fragment, a chromosome, a genome, a plasmid) as a duplication and/or a paralog.

Target nucleic acid can include microRNAs. MicroRNAs, miRNAs, or small temporal RNAs (stRNAs) are short (e.g., about 21 to 23 nucleotides long) and single-stranded RNA sequences involved in gene regulation. MicroRNAs may interfere with translation of messenger RNAs and are partially complementary to messenger RNAs. Target nucleic acid can include microRNA precursors such as primary transcript (pri-miRNA) and pre-miRNA stem-loop-structured RNA that is further processed into miRNA. Target nucleic acid can include short interfering RNAs (siRNAs), which are short (e.g., about 20 to 25 nucleotides long) and at least partially double-stranded RNA molecules involved in RNA interference (e.g., down-regulation of viral replication or gene expression).

Nucleic acid utilized in methods described herein can be obtained from any suitable biological specimen or sample, e.g., isolated from a sample obtained from a subject. A subject can be any living or non-living organism, including but not limited to a human, a non-human animal, a plant, a bacterium, a fungus, a virus, or a protist. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female, and a subject may be any age (e.g., an embryo, a fetus, infant, child, adult).

A sample or test sample can be any specimen that is isolated or obtained from a subject or part thereof. Non-limiting examples of specimens include fluid or tissue from a subject, including, without limitation, blood or a blood product (e.g., serum, plasma, or the like), umbilical cord blood, bone marrow, chorionic villi, amniotic fluid, cerebrospinal fluid, spinal fluid, lavage fluid (e.g., bronchoalveolar, gastric, peritoneal, ductal, ear, arthroscopic), serum, plasma, urine, aspirate, biopsy sample, celocentesis sample, cells (e.g., blood cells) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, hard tissues (e.g., liver, spleen, kidney, lung, or ovary), the like or combinations thereof. The term blood encompasses whole blood, blood product or any fraction of blood, such as serum, plasma, buffy coat, or the like as conventionally defined. Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to or after preparation.

A sample can include samples containing spores, viruses, cells, nucleic acids from prokaryotes or eukaryotes, and/or any free nucleic acid. For example, a method described herein can be used for detecting nucleic acid on the outside of spores (e.g., without the need for lysis). A sample can be isolated from any material suspected of containing a target sequence, such as from a subject described above. In some embodiments, a target sequence is present in air, plant, soil, or other materials suspected of containing biological organisms.

Nucleic acid can be derived (e.g., isolated, extracted, purified) from one or more sources by methods known in the art. Any suitable method can be used for isolating, extracting and/or purifying nucleic acid from a biological sample, including methods of DNA preparation in the art, and various commercially available reagents or kits, such as Qiagen's QIAamp Circulating Nucleic Acid Kit, QiaAmp DNA Mini Kit or QiaAmp DNA Blood Mini Kit (Qiagen, Hilden, Germany), GenomicPrep™ Blood DNA Isolation Kit (Promega, Madison, Wis.), GFX™ Genomic Blood DNA Purification Kit (Amersham, Piscataway, N.J.), and the like or combinations thereof. U.S. Pat. No. 7,888,006 provides DNA purification methods and does not disclose the compositions (e.g., lysis buffers, protectants) and methods provided herein

In some embodiments, a cell lysis procedure is performed. Cell lysis can be performed prior to initiation of an amplification reaction described herein (e.g., to release DNA and/or RNA from cells for amplification). Cell lysis procedures and reagents are known in the art and may be performed by chemical (e.g., detergent, hypotonic solutions, enzymatic procedures, and the like, or combination thereof), physical (e.g., French press, sonication, and the like), or electrolytic lysis methods. For example, chemical methods generally employ lysing agents to disrupt cells and extract nucleic acids from the cells, followed by treatment with chaotropic salts. In some embodiments, cell lysis comprises use of detergents (e.g., ionic, nonionic, anionic, zwitterionic). In some embodiments, cell lysis comprises use of ionic detergents (e.g., sodium dodecyl sulfate (SDS), sodium lauryl sulfate (SLS), deoxycholate, cholate, sarkosyl). Physical methods such as freeze/thaw followed by grinding, the use of cell presses and the like also may be useful. High salt lysis procedures also may be used. For example, an alkaline lysis procedure may be utilized. The latter procedure traditionally incorporates the use of phenol-chloroform solutions, and an alternative phenol-chloroform-free procedure involving three solutions may be utilized. In the latter procedures, one solution can contain 15 mM Tris, pH 8.0; 10 mM EDTA and 100 μg/ml Rnase A; a second solution can contain 0.2N NaOH and 1% SDS; and a third solution can contain 3M KOAc, pH 5.5, for example. In some embodiments, a cell lysis buffer is used in conjunction with the methods and components described herein.

Nucleic acid can be provided for conducting methods described herein without processing of the sample(s) containing the nucleic acid. For example, nucleic acid can be provided for conducting amplification methods described herein without prior nucleic acid purification. In some embodiments, a target sequence is amplified directly from a sample (e.g., without performing any nucleic acid extraction, isolation, purification and/or partial purification steps). In some embodiments, nucleic acid is provided for conducting methods described herein after processing of the sample(s) containing the nucleic acid. For example, a nucleic acid can be extracted, isolated, purified, or partially purified from the sample(s). The term “isolated” generally refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” can refer to a nucleic acid removed from a subject (e.g., a human subject). An isolated nucleic acid can be provided with fewer non-nucleic acid components (e.g., protein, lipid, carbohydrate) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” generally refers to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components.

Nucleic acid may be provided for conducting methods described herein without modifying the nucleic acid. Modifications can include, for example, denaturation, digestion, nicking, unwinding, incorporation and/or ligation of heterogeneous sequences, addition of epigenetic modifications, addition of labels (e.g., radiolabels such as ³²P, ³³P, ¹²⁵I, or ³⁵S; enzyme labels such as alkaline phosphatase; fluorescent labels such as fluorescein isothiocyanate (FITC); or other labels such as biotin, avidin, digoxigenin, antigens, haptens, fluorochromes), and the like. Accordingly, in some embodiments, an unmodified nucleic acid is amplified.

Methods disclosed herein for detecting a target nucleic acid sequence (single-stranded or ds DNA and/or RNA) in a sample can detect a target nucleic acid sequence (e.g., DNA or RNA) with a high degree of sensitivity. In some embodiments, the method can be used to detect a target DNA/RNA present in a sample comprising a plurality of RNAs/DNAs (including the target RNA/DNA and a plurality of non-target RNAs/DNAs), wherein the target RNA/DNA is present at one or more copies per 10, 20, 25, 50, 100, 500, 10³, 5×10³, 10⁴, 5×10⁴, 10⁵, 5×10⁵, 10⁶, or 10⁷, non-target DNAs/RNAs. As used herein, the terms “RNA/DNA” and “RNAs/DNAs” shall be given their ordinary meaning, and shall also refer to DNA, or RNA, or a combination of DNA and RNA.

The threshold of detection, for a method of detecting a target RNA/DNA in a sample, can be, for example 10 nM or less. The term “threshold of detection” shall be given its ordinary meaning, and shall also describe the minimal amount of target RNA/DNA that must be present in a sample in order for detection to occur. As an illustrative example, when a threshold of detection is 10 nM, then a signal can be detected when a target RNA/DNA is present in the sample at a concentration of 10 nM or more. In some embodiments, a disclosed method has a threshold of detection of 5 nM or less, 1 nM or less, 0.5 nM or less, 0.1 nM or less, 0.05 nM or less, 0.01 nM or less, 0.005 nM or less, 0.001 nM or less, 0.0005 nM or less, 0.0001 nM or less, 0.00005 nM or less, 0.00001 nM or less, 10 pM or less, 1 pM or less, 500 fM or less, 250 fM or less, 100 fM or less, 50 fM or less, 500 aM (attomolar) or less, 250 aM or less, 100 aM or less, 50 aM or less, 10 aM or less, or 1 aM or less. In some embodiments, a disclosed composition or method exhibits an attamolar (aM), femtomolar (fM), picomolar (pM), and/or nanomolar (nM), sensitivity of detection.

A sample can comprise sample nucleic acids (e.g., a plurality of sample nucleic acids). The term “plurality” is used herein to mean two or more. Thus, in some embodiments, a sample includes two or more (e.g., 3 or more, 5 or more, 10 or more, 20 or more, 50 or more, 100 or more, 500 or more, 1,000 or more, or 5,000 or more) sample nucleic acids (e.g., DNAs/RNAs). A disclosed method can be used as a very sensitive way to detect a target nucleic acid present in a sample (e.g., in a complex mixture of nucleic acids such as DNAs/RNAs). In some embodiments the sample includes 5, 10, 20, 25, 50, 100, 500, 10³, 5×10³, 10⁴, 5×10⁴, 10⁵, 5×10⁵, 10⁶, or 10⁷, 50, or more, DNAs/RNAs that differ from one another in sequence. In some embodiments, the sample includes DNAs/RNAs from a cell (e.g., a eukaryotic cell, a mammalian cell, or a human cell) or a cell lysate (e.g., a eukaryotic cell lysate, a mammalian cell lysate, a human cell lysate, a prokaryotic cell lysate, a plant cell lysate, or the like).

The term “sample” is used here shall be given its ordinary meaning and shall include any sample that includes RNA and/or DNA (e.g., in order to determine whether a target DNA and/or target RNA is present among a population of RNAs and/or DNAs). The sample can be derived from any source, e.g., the sample can be a synthetic combination of purified DNAs and/or RNAs; the sample can be a cell lysate, an DNA/RNA-enriched cell lysate, or DNAs/RNAs isolated and/or purified from a cell lysate. The sample can be from a patient (e.g., for the purpose of diagnosis). The sample can be from permeabilized cells, crosslinked cells, tissue sections, or combination thereof. The sample can be from tissues prepared by crosslinking followed by delipidation and adjustment to make a uniform refractive index. A sample can include a target nucleic acid (e.g., target DNA/RNA) and a plurality of non-target DNAs/RNAs. In some embodiments, the target DNA/RNA is present in the sample at one copy per 10, 20, 25, 50, 100, 500, 103, 5×10³, 10⁴, 5×10⁴, 10⁵, 5×10⁵, 10⁶, or 10⁷, non-target DNAs/RNAs.

A sample with respect to a patient encompasses blood and other liquid samples of biological origin, solid tissue samples such as a biopsy specimen or tissue cultures or cells derived therefrom and the progeny thereof, as well as samples that have been manipulated in any way after their procurement (such as by treatment with reagents); washed; or enriched for certain cell populations (e.g., cancer cells) or particular types of molecules (e.g., RNAs). A sample can comprise, or be, a biological sample including but not limited to a clinical sample such as blood, plasma, serum, aspirate, cerebral spinal fluid (CSF), and also includes tissue obtained by surgical resection, tissue obtained by biopsy, cells in culture, cell supernatants, cell lysates, tissue samples, organs, bone marrow, and the like. A biological sample can comprise biological fluids derived therefrom (e.g., cancerous cell, infected cell, etc.), e.g., a sample comprising RNAs that is obtained from such cells (e.g., a cell lysate or other cell extract comprising RNAs).

The source of the sample can be a (or is suspected of being a) diseased cell, fluid, tissue, or organ; or a normal (non-diseased) cell, fluid, tissue, or organ. In some embodiments, the source of the sample is a (or is suspected of being a) pathogen-infected cell, tissue, or organ. For example, the source of a sample can be an individual who may or may not be infected- and the sample can be any biological sample (e.g., blood, saliva, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, an epithelial cell sample (e.g., epithelial cell scraping), etc.) collected from the individual. The sample can be a cell-free liquid sample or a liquid sample that comprise cells. Pathogens can be viruses, fungi, helminths, protozoa, malarial parasites, Plasmodium parasites, Toxoplasma parasites, Schistosoma parasites, and the like. “Helminths” include roundworms, heartworms, and phytophagous nematodes (Nematoda), flukes (Tematoda), Acanthocephala, and tapeworms (Cestoda). Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, Plasmodium vivax, Trypanosoma cruzi and Toxoplasma gondii. Fungal pathogens include, but are not limited to: Cryptococcus neoformans, Histoplasma capsulatum, Coccidioides immitis, Blastomyces dermatitidis, Chlamydia trachomatis, and Candida albicans. Pathogenic viruses include, e.g., immunodeficiency virus (e.g., HIV); influenza virus; dengue; West Nile virus; herpes virus; yellow fever virus; Hepatitis C virus; Hepatitis A virus; Hepatitis B virus; papillomavirus; and the like. Pathogenic viruses can include DNA viruses such as: a papovavirus (e.g., HPV, polyomavirus); a hepadnavirus; a herpesvirus (e.g., HSV (e.g., HSV I, HSV II), varicella zoster virus (VZV), epstein-barr virus (EBV), cytomegalovirus (CMV), herpes lymphotropic virus, Pityriasis Rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. Non-limiting examples of pathogens include Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, human serum parvo-like virus, respiratory syncytial virus, measles virus, adenovirus, human T-cell leukemia viruses, murine leukemia virus, mumps virus, vesicular stomatitis virus, Sindbis virus, lymphocytic choriomeningitis virus, wart virus, blue tongue virus, Sendai virus, feline leukemia virus, reovirus, polio virus, simian virus 40, mouse mammary tumor virus, dengue virus, rubella virus, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae.

### Amplification

Provided herein are methods for amplifying nucleic acid. In some embodiments, nucleic acids are amplified using a suitable amplification process. Nucleic acid amplification typically involves enzymatic synthesis of nucleic acid amplicons (copies), which contain a sequence complementary to a nucleotide sequence being amplified. In some embodiments, an amplification method is performed in a single vessel, a single chamber, and/or a single volume (i.e., contiguous volume). In some embodiments, an amplification method and a detection method (e.g., a detection method described herein) are performed in a single vessel, a single chamber, and/or a single volume (i.e., contiguous volume).

The terms “amplify”, “amplification”, “amplification reaction”, or “amplifying” refer to any in vitro process for multiplying the copies of a target nucleic acid. Amplification sometimes refers to an “exponential” increase in target nucleic acid. “Amplifying” can also refer to linear increases in the numbers of a target nucleic acid, but is different than a one-time, single primer extension step. In some embodiments a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may limit inaccuracies associated with depleted reactants in certain amplification reactions, and also may reduce amplification biases due to nucleotide sequence or species abundance of the target. In some embodiments a one-time primer extension may be performed as a prelude to linear or exponential amplification.

A generalized description of an amplification process is presented herein. Primers (e.g., oligonucleotides described herein) and target nucleic acid are contacted, and complementary sequences anneal or hybridize to one another, for example. Primers can anneal to a target nucleic acid, at or near (e.g., adjacent to, abutting, and the like) a sequence of interest. A primer annealed to a target may be referred to as a primer-target hybrid, hybridized primer-target, or a primer-target duplex. The terms near or adjacent to when referring to a nucleotide sequence of interest refer to a distance (e.g., number of bases) or region between the end of the primer and the nucleotide or nucleotides (e.g., nucleotide sequence) of a target. Generally, adjacent is in the range of about 1 nucleotide to about 50 nucleotides (e.g., about 1, 2, 3, 4, 5, 6, 7, 8, 9, 10, 20, 30, 40, or 50 nucleotide(s)) away from a nucleotide or nucleotide sequence of interest. In some embodiments, primers in a set (e.g., a pair of primers, a forward and a reverse primer, a first oligonucleotide and a second oligonucleotide) anneal within about 1 to 20 nucleotides from a nucleotide or nucleotide sequence of interest and produce amplified products. In some embodiments, primers anneal within a nucleotide or a nucleotide sequence of interest. After annealing, each primer is extended along the target (i.e., template strand) by a polymerase to generate a complementary strand. Several cycles of primer annealing and extension can be carried out, for example, until a detectable amount of amplification product is generated. In some embodiments, where a target nucleic acid is RNA, a DNA copy (cDNA) of the target RNA is synthesized prior to or during the amplification step by reverse transcription.

Components of an amplification reaction (e.g., the one or more amplification reagents) can include, for example, one or more primers (e.g., individual primers, primer pairs, primer sets, oligonucleotides, multiple primer sets for multiplex amplification, and the like), nucleic acid target(s) (e.g., target nucleic acid from a sample), one or more polymerases, nucleotides (e.g., dNTPs and the like), and a suitable buffer (e.g., a buffer comprising a detergent, a reducing agent, monovalent ions, and divalent ions). An amplification reaction can further include one or more of: a reverse transcriptase, a reverse transcription primer, and one or more detection agents.

Nucleic acid amplification can be conducted in the presence of native nucleotides, for example, dideoxyribonucleoside triphosphates (dNTPs), and/or derivatized nucleotides. A native nucleotide generally refers to adenylic acid, guanylic acid, cytidylic acid, thymidylic acid, or uridylic acid. A derivatized nucleotide generally is a nucleotide other than a native nucleotide. A ribonucleoside triphosphate is referred to as NTP or rNTP, where N can be A, G, C, U. A deoxynucleoside triphosphate substrates is referred to as dNTP, where N can be A, G, C, T, or U. Monomeric nucleotide subunits may be denoted as A, G, C, T, or U herein with no particular reference to DNA or RNA. In some embodiments, non-naturally occurring nucleotides or nucleotide analogs, such as analogs containing a detectable label (e.g., fluorescent or colorimetric label), may be used. For example, nucleic acid amplification can be carried out in the presence of labeled dNTPs, for example, radiolabels such as ³²P, ³³P, ¹²⁵I, or ³⁵S; enzyme labels such as alkaline phosphatase; fluorescent labels such as fluorescein isothiocyanate (FITC); or other labels such as biotin, avidin, digoxigenin, antigens, haptens, or fluorochromes. In some embodiments, nucleic acid amplification may be carried out in the presence of modified dNTPs, for example, heat activated dNTPs (e.g., CleanAmp™ dNTPs from TriLink).

The one or more amplification reagents can include non-enzymatic components and enzymatic components. Non-enzymatic components can include, for example, primers, nucleotides, buffers, salts, reducing agents, detergents, and ions. In some embodiments, the Non-enzymatic components do not include proteins (e.g., nucleic acid binding proteins), enzymes, or proteins having enzymatic activity, for example, polymerases, reverse transcriptases, helicases, topoisomerases, ligases, exonucleases, endonucleases, restriction enzymes, nicking enzymes, recombinases and the like. In some embodiments, an enzymatic component consists of a polymerase or consists of a polymerase and a reverse transcriptase. Accordingly, such enzymatic components would exclude other proteins (e.g., nucleic acid binding proteins and/or proteins having enzymatic activity), for example, helicases, topoisomerases, ligases, exonucleases, endonucleases, restriction enzymes, nicking enzymes, recombinases, and the like.

In some embodiments, amplification conditions comprise an enzymatic activity (e.g., an enzymatic activity provided by a polymerase or provided by a polymerase and a reverse transcriptase). In some embodiments, the enzymatic activity does not include enzymatic activity provided by enzymes other than the polymerase and/or the reverse transcriptase, for example, helicases, topoisomerases, ligases, exonucleases, endonucleases, restriction enzymes, nicking enzymes, recombinases, and the like. A polymerase activity and a reverse transcriptase activity can be provided by separate enzymes or separate enzyme types (e.g., polymerase(s) and reverse transcriptase(s)), or provided by a single enzyme or enzyme type (e.g., polymerase(s)).

Amplification of nucleic acid can comprise a non-thermocycling type of PCR. In some embodiments, amplification of nucleic acid comprises an isothermal amplification process, for example an isothermal polymerase chain reaction (iPCR). Isothermal amplification generally is an amplification process performed at a constant temperature. Terms such as isothermal conditions, isothermally and constant temperature generally refer to reaction conditions where the temperature of the reaction is kept essentially constant during the course of the amplification reaction. Isothermal amplification conditions generally do not include a thermocycling (i.e., cycling between an upper temperature and a lower temperature) component in the amplification process. When amplifying under isothermal conditions, the reaction can be kept at an essentially constant temperature, which means the temperature may not be maintained at precisely one temperature. For example, small fluctuations in temperature (e.g., ±1 to 5° C.) may occur in an isothermal amplification process due to, for example, environmental or equipment-based variables. Often, the entire reaction volume is kept at an essentially constant temperature, and isothermal reactions herein generally do not include amplification conditions that rely on a temperature gradient generated within a reaction vessel and/or convective-flow based temperature cycling.

Isothermal amplification reactions herein can be conducted at an essentially constant temperature. In some embodiments, isothermal amplification reactions herein are conducted at a temperature of about 55° C. to a temperature of about 75° C., for example at a temperature of, or a temperature of about, 55, 56, 57, 58, 59, 60, 61, 62, 63, 64, 65, 66, 67, 68, 69, 70, 71, 72, 73, 74, or about 75° C., or a number or a range between any two of these values. In some embodiments, a temperature element (e.g., heat source) is kept at an essentially constant temperature, for example an essentially constant temperature at or below about 75° C., at or below about 70 degrees Celsius, at or below about 65° C., or at or below about 60° C.

An amplification process herein can be conducted over a certain length of time, for example until a detectable nucleic acid amplification product is generated. A nucleic acid amplification product may be detected by any suitable detection process and/or a detection process described herein. The amplification process can be conducted over a length of time within about 20 minutes or less, or about 10 minutes or less. For example, an amplification process can be conducted within about 1, 2, 3, 4, 5, 6, 7, 8, 9, 10, 11, 12, 13, 14, 15, 16, 17, 18, 19 or 20 minutes, or a number or a range between any two of these values.

Nucleic acid targets can be amplified without exposure to agents or conditions that denature nucleic acid, in some embodiments. Nucleic acid targets can be amplified without exposure to agents or conditions that promote strand separation during the amplification step (and/or other steps) in some embodiments. Nucleic acid targets can be amplified without exposure to agents or conditions that promote unwinding during the amplification step (and/or other steps) in some embodiments. Agents or conditions that denature nucleic acid and/or promote strand separation and/or promote unwinding may include, for example, thermal conditions (e.g., high temperatures), pH conditions (e.g., high or low pH), chemical agents, proteins (e.g., enzymatic agents), and the like.

In some embodiments, the methods disclosed herein does not comprise thermal denaturation (e.g., heating a solution containing a nucleic acid to an elevated temperature, such as, for example a temperature above 75° C., 80° C., 90° C., or 95° C., or higher) or protein-based (e.g., enzymatic) denaturation of a nucleic acid. Protein-based (e.g., enzymatic) denaturation can comprise contacting a nucleic acid with one or more of a helicase, a topoisomerase, a ligase, an exonuclease, an endonuclease, a restriction enzyme, a nicking enzyme, a recombinase, a RNA replicase, and a nucleic acid binding protein (e.g., single-stranded binding protein). In some embodiments, the compositions provided herein do not comprise a helicase, a topoisomerase, a ligase, an exonuclease, an endonuclease, a restriction enzyme, a nicking enzyme, a recombinase, a RNA replicase, and/or a nucleic acid binding protein (e.g., single-stranded binding protein). In some embodiments, the compositions and methods provided herein do not comprise intercalators, alkylating agents, and/or chemicals such as formamide, glycerol, urea, dimethyl sulfoxide (DMSO), or N,N,N-trimethylglycine (betaine). In some embodiments, the disclosed methods do not comprise contacting a nucleic acid with denaturing agents (e.g., formamide). In some embodiments, the amplifying step does not comprise agents and/or conditions that denature nucleic acids (e.g., promote strand separation and/or promote unwinding). In some embodiments, the amplifying step (e.g., step (c)) does not comprise agents and/or conditions that denature nucleic acids (e.g., promote strand separation and/or promote unwinding) other than a polymerase (e.g., a hyperthermophile polymerase). In some embodiments, the methods and compositions provided herein not comprise agents and/or conditions that denature nucleic acids (e.g., promote strand separation and/or promote unwinding) other than a polymerase (e.g., a hyperthermophile polymerase) and/or low pH conditions (e.g., contact with acid(s)).

Nucleic acid targets can be amplified without exposure to agents or conditions that promote strand separation and/or unwinding, for example a helicase, a topoisomerase, a ligase, an exonuclease, an endonuclease, a restriction enzyme, a nicking enzyme, a recombinase, a RNA replicase, a nucleic acid binding protein (e.g., single-stranded binding protein), or any combination thereof. For example, nucleic acid targets can be amplified without exposure to a helicase, including but not limited to DNA helicases and RNA helicases. Amplification conditions that do not include use of a helicase are helicase-free amplification conditions.

Nucleic acid targets can be amplified without exposure to a recombinase, including but not limited to, Cre recombinase, Hin recombinase, Tre recombinase, FLP recombinase, RecA, RAD51, RadA, T4 uvsX. In some embodiments, nucleic acid targets are amplified without exposure to a recombinase accessory protein, for example, a recombinase loading factor (e.g., T4 uvsY). Nucleic acid targets can be amplified without exposure to a nucleic acid binding protein (e.g., single-stranded binding protein or single-strand DNA-binding protein (SSB)), for example, T4 gp32. In some embodiments, nucleic acid targets are amplified without exposure to a topoisomerase. Nucleic acid targets can be amplified with or without exposure to agents or conditions that destabilize nucleic acid. As used herein, the term “destabilization” shall be given its ordinary meaning, and shall also refer to a disruption in the overall organization and geometric orientation of a nucleic acid molecule (e.g., double helical structure) by one or more of tilt, roll, twist, slip, and flip effects (e.g., as described in Lenglet et al., (2010) Journal of Nucleic Acids Volume 2010, Article ID 290935, 17 pages). Destabilization generally does not refer to melting or separation of nucleic acid strands (e.g., denaturation). Nucleic acid destabilization can be achieved, for example, by exposure to agents such as intercalators or alkylating agents, and/or chemicals such as formamide, urea, dimethyl sulfoxide (DMSO), or N,N,N-trimethylglycine (betaine). In some embodiments, methods provided herein include use of one or more destabilizing agents. In some embodiments, methods provided herein exclude use of destabilizing agents. In some embodiments, nucleic acid targets are amplified without exposure to a ligase and/or an RNA replicase.

Nucleic acid targets can be amplified without cleavage or digestion, in some embodiments. For example, nucleic acid targets can be amplified without prior exposure to one or more cleavage agents, and intact nucleic acid is amplified. In some embodiments, nucleic acid targets are amplified without exposure to one or more cleavage agents during amplification. In some embodiments, nucleic acid targets are amplified without exposure to one or more cleavage agents after amplification. Amplification conditions that do not include use of a cleavage agent may be referred to herein as cleavage agent-free amplification conditions. The term “cleavage agent” generally refers to an agent, sometimes a chemical or an enzyme that can cleave a nucleic acid at one or more specific or non-specific sites. Specific cleavage agents often cleave specifically according to a particular nucleotide sequence at a particular site. Cleavage agents can include endonucleases (e.g., restriction enzymes, nicking enzymes, and the like); exonucleases (DNAses, RNAses (e.g., RNAseH), 5′ to 3′ exonucleases (e.g. exonuclease II), 3′ to 5′ exonucleases (e.g. exonuclease I), and poly(A)-specific 3′ to 5′ exonucleases); and chemical cleavage agents.

Nucleic acid targets can be amplified without use of restriction enzymes and/or nicking enzymes. In some embodiments, nucleic acid is amplified without prior exposure to restriction enzymes and/or nicking enzymes. In some embodiments, nucleic acid is amplified without exposure to restriction enzymes and/or nicking enzymes during amplification. In some embodiments, nucleic acid is amplified without exposure to restriction enzymes and/or nicking enzymes after amplification. Nucleic acid targets can be amplified without exonuclease treatment. Exonucleases include, for example, DNAses, RNAses (e.g., RNAseH), 5′ to 3′ exonucleases (e.g. exonuclease II), 3′ to 5′ exonucleases (e.g. exonuclease I), and poly(A)-specific 3′ to 5′ exonucleases. In some embodiments, nucleic acid is amplified without exonuclease treatment prior to, during, and/or after amplification. Amplification conditions that do not include use of an exonuclease are exonuclease-free amplification conditions. In some embodiments, nucleic acid is amplified without DNAse treatment and/or RNAse treatment. In some embodiments, nucleic acid is amplified without RNAseH treatment.

An amplified nucleic acid may be referred to herein as a nucleic acid amplification product or amplicon. In some embodiments, the amplification product includes naturally occurring nucleotides, non-naturally occurring nucleotides, nucleotide analogs and the like and combinations of the foregoing. An amplification product typically has a nucleotide sequence that is identical to or substantially identical to a sequence in a sample nucleic acid (e.g., target sequence) or complement thereof. A “substantially identical” nucleotide sequence in an amplification product will generally have a high degree of sequence identity to the nucleotide sequence being amplified or complement thereof (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% sequence identity), and variations sometimes are a result of polymerase infidelity or other variables.

In some embodiments, a nucleic acid amplification product comprises a polynucleotide that is continuously complementary to or substantially identical to a target sequence in sample nucleic acid. Continuously complementary generally refers to a nucleotide sequence in a first strand, for example, where each base in order (e.g., read 5′ to 3′) pairs with a correspondingly ordered base in a second strand, and there are no gaps, additional sequences or unpaired bases within the sequence considered as continuously complementary. Stated another way, continuously complementary generally refers to all contiguous bases of a nucleotide sequence in a first stand being complementary to corresponding contiguous bases of a nucleotide sequence in a second strand. For example, a first strand having a sequence 5′-ATGCATGCATGC-3′ (SEQ ID NO: 3) would be considered as continuously complementary to a second strand having a sequence 5′-GCATGCATGCAT-3′ (SEQ ID NO: 4), where all contiguous bases in the first strand are complementary to all corresponding contiguous bases in the second strand. However, a first strand having a sequence 5′-ATGCATAAAAAAGCATGC-3′ (SEQ ID NO: 5) would not be considered as continuously complementary to a second strand having a sequence 5′-GCATGCATGCAT-3′ (SEQ ID NO: 4), because the sequence of six adenines (6 As) in the middle of the first strand would not pair with bases in the second strand. A continuously complementary sequence sometimes is about 5 to about 25 contiguous bases in length, e.g., about 5, 6, 7, 8, 9, 10, 11, 12, 13, 14, 15, 16, 17, 18, 19, 20, 21, 22, 23, 24, 25, or a range between any two of these values, contiguous bases in length. In some embodiments, a nucleic acid amplification product consists of a polynucleotide that is continuously complementary to or substantially identical to a target sequence in sample nucleic acid. Accordingly, in some embodiments, a nucleic acid amplification product does not include any additional sequences (e.g., at the 5′ and/or 3′ end, or within the product) that are not continuously complementary to or substantially identical to a target sequence, for example, additional sequences incorporated into an amplification product by way of tailed primers or ligation, and/or additional sequences providing cleavage agent recognition sites (e.g., nicking enzyme recognition sites). Generally, unless a target sequence comprises tandem repeats, an amplification product does not include product in the form of tandem repeats.

Nucleic acid amplification products can comprise sequences complementary to or substantially identical to one or more primers used in an amplification reaction. In some embodiments, a nucleic acid amplification product comprises a first nucleotide sequence that is continuously complementary to or identical to a first primer sequence, and a second nucleotide sequence that is continuously complementary to or identical to a second primer sequence.

Nucleic acid amplification products can comprise a spacer sequence. As described herein, a spacer sequence in an amplification product is a sequence (1 or more bases) continuously complementary to or substantially identical to a portion of a target sequence in the sample nucleic acid, and is flanked by sequences in the amplification product that are complementary to or substantially identical to one or more primers used in an amplification reaction. A spacer sequence flanked by sequences in the amplification product generally lies between a first sequence (complementary to or substantially identical to a first primer) and a second sequence (complementary to or substantially identical to a second primer). Thus, an amplification product typically includes a first sequence followed by a spacer sequences followed by a second sequence. A spacer sequence generally is not complementary to or substantially identical to a sequence in the primer(s). A spacer sequence can be, or can comprise, about 1 to 10 bases, including 1, 2, 3, 4, 5, 6, 7, 8, 9, or 10 bases. In some embodiments, a nucleic acid amplification product consists of, or consists essentially of, a first nucleotide sequence that is continuously complementary to or identical to a first primer sequence, a second nucleotide sequence that is continuously complementary to or identical to a second primer sequence, and a spacer sequence. In some embodiments, a nucleic acid amplification product does not include any additional sequences (e.g., at the 5′ and/or 3′ end; or within the product) that are not continuously complementary to or identical to a first primer sequence and a second primer sequence, and are not part of a spacer sequence, for example, additional sequences incorporated into an amplification product by way of tailed or looped primers, ligation or other mechanism. In some embodiments, a nucleic acid amplification product generally does not include additional sequences (e.g., at the 5′ and/or 3′ end; or within the product) that are not continuously complementary to or identical to a first primer sequence and a second primer sequence, and are not part of a spacer sequence, for example, additional sequences incorporated into an amplification product by way of tailed or looped primers, ligation or other mechanism. However, in such embodiments, a nucleic acid amplification product may include, for example, some mismatched (i.e., non-complementary) bases or one more extra bases (e.g., at the 5′ and/or 3′ end; or within the product) introduced into the product by way of error or promiscuity in the amplification process.

Nucleic acid amplification products can be up to 50 bases in length, including 10, 15, 20, 25, 30, 35, 40, 45, 50, or a number or a range between any two of these values, bases long. In some embodiments, nucleic acid amplification products for a given target sequence have the same length or substantially the same length (e.g., within 1 to 10 bases). Accordingly, nucleic acid amplification products for a given target sequence may produce a single signal (e.g., band on an electrophoresis gel) and generally do not produce multiple signals indicative of multiple lengths (e.g., a ladder or smear on an electrophoresis gel). For multiplex reactions, nucleic acid amplification products for different target sequences may have different lengths.

The methods and components described herein can be used for multiplex amplification which generally refers to the amplification of more than one nucleic acid of interest (e.g., amplification or more than one target sequence). For example, multiplex amplification can refer to amplification of multiple sequences from the same sample or amplification of one of several sequences in a sample. Multiplex amplification also can refer to amplification of one or more sequences present in multiple samples either simultaneously or in step-wise fashion. For example, a multiplex amplification can be used for amplifying least two target sequences that are capable of being amplified (e.g., the amplification reaction comprises the appropriate primers and enzymes to amplify at least two target sequences). In some embodiments, an amplification reaction is prepared to detect at least two target sequences, but only one of the target sequences is present in the sample being tested, such that both sequences are capable of being amplified, but only one sequence is amplified. In some embodiments, where two target sequences are present, an amplification reaction results in the amplification of both target sequences. A multiplex amplification reaction can result in the amplification of one, some, or all of the target sequences for which it comprises the appropriate primers and enzymes. In some embodiments, an amplification reaction is prepared to detect two sequences with one pair of primers, where one sequence is a target sequence and one sequence is a control sequence (e.g., a synthetic sequence capable of being amplified by the same primers as the target sequence and having a different spacer base or sequence than the target). In some embodiments, an amplification reaction is prepared to detect multiple sets of sequences with corresponding primer pairs, where each set includes a target sequence and a control sequence.

### Primers

Nucleic acid amplification generally is conducted in the presence of one or more primers. A primer is generally characterized as an oligonucleotide that includes a nucleotide sequence capable of hybridizing or annealing to a target nucleic acid, at or near (e.g., adjacent to) a specific region of interest (i.e., target sequence). Primers can allow for specific determination of a target nucleic acid nucleotide sequence or detection of the target nucleic acid (e.g., presence or absence of a sequence), or feature thereof, for example. A primer can be naturally occurring or synthetic. The term specific, or specificity, generally refers to the binding or hybridization of one molecule to another molecule, such as a primer for a target polynucleotide. That is, specific or specificity refers to the recognition, contact, and formation of a stable complex between two molecules, as compared to substantially less recognition, contact, or complex formation of either of those two molecules with other molecules. The term anneal or hybridize generally refers to the formation of a stable complex between two molecules. The terms primer, oligo, or oligonucleotide may be used interchangeably herein, when referring to primers.

A primer can be designed and synthesized using suitable processes, and can be of any length suitable for hybridizing to a target sequence and performing an amplification process described herein. Primers often are designed according to a sequence in a target nucleic acid. A primer in some embodiments may be about 5 to about 30 bases in length, for example 5, 6, 7, 8, 9, 10, 11, 12, 13, 14, 15, 16, 17, 18, 19, 20, 21, 22, 23, 24, 25, 26, 27, 28, 29 or 30 bases in length. A primer may be composed of naturally occurring and/or non-naturally occurring nucleotides (e.g., modified nucleotides, labeled nucleotides), or a mixture thereof. Modifications and modified bases may include, for example, phosphorylation, (e.g., 3′ phosphorylation, 5′ phosphorylation); attachment chemistry or linkers modifications (e.g., Acrydite™, adenylation, azide (NHS ester), digoxigenin (NHS ester), cholesteryl-TEG, I-Linker™, amino modifiers (e.g., amino modifier C6, amino modifier C12, amino modifier C6 dT, Uni-Link™ amino modifier), alkynes (e.g., 5′ hexynyl, 5-octadiynyl dU), biotinylation (e.g., biotin, biotin (azide), biotin dT, biotin-TEG, dual biotin, PC biotin, desthiobiotin-TEG), thiol modifications (e.g., thiol modifier C3 S—S, dithiol, thiol modifier C6 S—S)); fluorophores (e.g., Freedom™ Dyes, Alexa Fluor® Dyes, LI-COR IRDyes®, ATTO™ Dyes, Rhodamine Dyes, WellRED Dyes, 6-FAM (azide), Texas Red®-X (NHS ester), Lightcycler® 640 (NHS ester), Dy 750 (NHS ester)); Iowa Black® dark quenchers modifications (e.g., Iowa Black® FQ, Iowa Black® RQ); dark quenchers modifications (e.g., Black Hole Quencher®-1, Black Hole Quencher®-2, Dabcyl); spacers (C3 spacer, PC spacer, hexanediol, spacer 9, spacer 18, 1′,2′-dideoxyribose (dSpacer); modified bases (e.g., 2-aminopurine, 2,6-diaminopurine (2-amino-dA), 5-bromo dU, deoxyUridine, inverted dT, inverted dideoxy-T, dideoxy-C, 5-methyl dC, deoxylnosine, Super T®, Super G®, locked nucleic acids (LNA's), 5-nitroindole, 2′-O-methyl RNA bases, hydroxmethyl dC, UNA unlocked nucleic acid (e.g., UNA-A, UNA-U, UNA-C, UNA-G), Iso-dC, Iso-dG, Fluoro C, Fluoro U, Fluoro A, Fluoro G); phosphorothioate (PS) bonds modifications (e.g., phosphorothioated DNA bases, phosphorothioated RNA bases, phosphorothioated 2′ O-methyl bases, phosphorothioated LNA bases); and click chemistry modifications. In some embodiments, modifications and modified bases include uracil bases, ribonucleotide bases, O-methyl RNA bases, PS linkages, 3′ phosphate groups, spacer bases (such as C3 spacer or other spacer bases). For example, a primer may comprise one or more O-methyl RNA bases (e.g., 2′-O-methyl RNA bases). 2′-O-methyl RNA generally is a post-transcriptional modification of RNA found in tRNA and other small RNAs. Primers can be directly synthesized that include 2′-O-methyl RNA bases. This modification can, for example, increase Tm of RNA:RNA duplexes and provide stability in the presence of single-stranded ribonucleases and DNases. 2′-O-methyl RNA bases may be included in primers, for example, to increase stability and binding affinity to a target sequence. In some embodiments, a primer may comprise one or more phosphorothioate (PS) linkages (e.g., PS bond modifications). A PS bond substitutes a sulfur atom for a non-bridging oxygen in the phosphate backbone of a primer. This modification typically renders the internucleotide linkage resistant to nuclease degradation. PS bonds can be introduced between about the last 3 to 5 nucleotides at the 5′-end or the 3′-end of a primer to inhibit exonuclease degradation, for example. PS bonds included throughout an entire primer can help reduce attack by endonucleases, in some embodiments. A primer can, for example, comprise a 3′ phosphate group. 3′ phosphorylation can inhibit degradation by certain 3′-exonucleases and can be used to block extension by DNA polymerases, in certain instances. In some embodiments, a primer comprises one or more spacer bases (e.g., one or more C3 spacers). A C3 spacer phosphoramidite can be incorporated internally or at the 5′-end of a primer. Multiple C3 spacers can be added at either end of a primer to introduce a long hydrophilic spacer arm for the attachment of fluorophores or other pendent groups, for example.

A primer can comprises DNA bases, RNA bases, or both, where one or more of the DNA bases and RNA bases is modified or unmodified. For example, a primer can be a mixture of DNA bases and RNA bases. The primer can consist of DNA bases (e.g., modified DNA bases and/or unmodified DNA bases). In some embodiments, the primer consists of unmodified DNA bases. In some embodiments, the primer consists of modified DNA bases. The primer can consist of RNA bases (e.g., modified RNA bases and/or unmodified RNA bases). In some embodiments, the primer consists of unmodified RNA bases. In some embodiments, the primer consists of modified RNA bases. In some embodiments, a primer comprises no RNA bases. In some embodiments, a primer comprises no DNA bases. In some embodiments, the primer comprises no cleavage agent recognition sites (e.g., no nicking enzyme recognition sites). In some embodiments, a primer comprises no tail (e.g., no tail comprising a nicking enzyme recognition site).

All or a portion of a primer sequence can be complementary or substantially complementary to a target nucleic acid, in some embodiments. Substantially complementary with respect to sequences generally refers to nucleotide sequences that will hybridize with each other. The stringency of the hybridization conditions can be altered to tolerate varying amounts of sequence mismatch. The target and primer sequences can be, for example, at least 75% complementary to each other, including 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to each other. Primers that are substantially complimentary to a target nucleic acid sequence typically are also substantially identical to the complement of the target nucleic acid sequence (i.e., the sequence of the anti-sense strand of the target nucleic acid). The primer and the anti-sense strand of the target nucleic acid can be at least 75% identical in sequence, for example 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each other.

In some embodiments, primers comprise a pair of primers. A pair of primers may include a forward primer and a reverse primer (e.g., primers that bind to the sense and antisense strands of a target nucleic acid). In some embodiments, primers consist of a pair of primers (i.e. a forward primer and a reverse primer). Accordingly, in some embodiments, amplification of a target sequence is performed using a pair of primers and no additional primers or oligonucleotides are included in the amplification of the target sequence (e.g., the amplification reaction components comprise no additional primer pairs for a given target sequence, no nested primers, no bumper primers, no oligonucleotides other than the primers, no probes, and the like). In some embodiments, primers consist of a pair of primers. In some embodiments, an amplification reaction can include additional primer pairs for amplifying different target sequences, such as in a multiplex amplification. In some embodiments, primers consist of a pair of primers, however, in some embodiments, an amplification reaction can include additional primers, oligonucleotides or probes for a detection process that are not considered part of amplification. In some embodiments, primers are used in sets. An amplification primer set can include a pair of forward and reverse primers for a given target sequence. For multiplex amplification, primers that amplify a first target sequence are considered a primer set, and primers that amplify a second target sequence are considered a different primer set.

Amplification reaction components can comprise, or consist of, a first primer (first oligonucleotide) complementary to a target sequence in a first strand (e.g., sense strand, forward strand) of a sample nucleic acid, and a second primer (second oligonucleotide) complementary to a target sequence in a second strand (e.g., antisense strand, reverse strand) of a sample nucleic acid. In some embodiments, a first primer (first oligonucleotide) comprises a first polynucleotide continuously complementary to a target sequence in a first strand of sample nucleic acid, and a second primer (second oligonucleotide) comprises a second polynucleotide continuously complementary to a target sequence in a second strand of sample nucleic acid. Continuously complementary for a primer-target generally refers to a nucleotide sequence in a primer, where each base in order pairs with a correspondingly ordered base in a target sequence, and there are no gaps, additional sequences or unpaired bases within the sequence considered as continuously complementary. In some embodiments, a primer does not include any additional sequences (e.g., at the 5′ and/or 3′ end, or within the primer) that are not continuously complementary to a target sequence, for example, additional sequences present in tailed primers or looped primers, and/or additional sequences providing cleavage agent recognition sites (e.g., nicking enzyme recognition sites). In some embodiments, amplification reaction components do not comprise primers comprising additional sequences (i.e., sequences other than the sequence that is continuously complementary to a target sequence), for example, tailed primers, looped primers, primers capable of forming step-loop structures, hairpin structures, and/or additional sequences providing cleavage agent recognition sites (e.g., nicking enzyme recognition sites), and the like.

The primer, in some embodiments, can contain a modification such as one or more inosines, abasic sites, locked nucleic acids, minor groove binders, duplex stabilizers (e.g., acridine, spermidine), Tm modifiers or any modifier that changes the binding properties of the primer. The primer, in some embodiments, can contain a detectable molecule or entity (e.g., a fluorophore, radioisotope, colorimetric agent, particle, enzyme and the like).

### Polymerase

Amplification reaction components (e.g., one or more amplification reagents) can comprise one or more polymerases. Polymerases are proteins capable of catalyzing the specific incorporation of nucleotides to extend a 3′ hydroxyl terminus of a primer molecule, for example, an amplification primer described herein, against a nucleic acid target sequence (e.g., to which a primer is annealed). Non-limiting examples of polymerases include thermophilic or hyperthermophilic polymerases that can have activity at an elevated reaction temperature (e.g., above 55, 60, 65, 70, 75, 80, 85, 90, 95, or 100° C.). A hyperthermophilic polymerase may be referred to as a hyperthermophile polymerase. A polymerase may or may not have strand displacement capabilities. In some embodiments, a polymerase can incorporate about 1 to about 50 nucleotides in a single synthesis, for example about 1, 5, 10, 15, 20, 25, 30, 35, 40, 45 or 50 nucleotides, or a number or a range between any two of these values, in a single synthesis.

The amplification reaction components can comprise one or more DNA polymerases selected from: 9° N DNA polymerase; 9° Nm™ DNA polymerase; Therminator™ DNA Polymerase; Therminator™ II DNA Polymerase; Therminator™ III DNA Polymerase; Therminator™ γ DNA Polymerase; Bst DNA polymerase; Bst DNA polymerase (large fragment); Phi29 DNA polymerase, DNA polymerase I (E. coli), DNA polymerase I, large (Klenow) fragment; Klenow fragment (3′-5′ exo-); T4 DNA polymerase; T7 DNA polymerase; Deep VentR™ (exo-) DNA Polymerase; Deep VentR™ DNA Polymerase; DyNAzyme™ EXT DNA; DyNAzyme™ II Hot Start DNA Polymerase; Phusion™ High-Fidelity DNA Polymerase; VentR® DNA Polymerase; VentR® (exo-) DNA Polymerase; RepliPHI™ Phi29 DNA Polymerase; rBst DNA Polymerase, large fragment (IsoTherm™ DNA Polymerase); MasterAmp™ AmpliTherm™ DNA Polymerase; Tag DNA polymerase; Tth DNA polymerase; Tfl DNA polymerase; Tgo DNA polymerase; SP6 DNA polymerase; Tbr DNA polymerase; DNA polymerase Beta; and ThermoPhi DNA polymerase.

In some embodiments, the amplification reaction components comprise one or more hyperthermophile DNA polymerases (e.g., hyperthermophile DNA polymerases that are thermostable at high temperatures). The hyperthermophile DNA polymerase can have a half-life of about 5 to 10 hours at 95° C. and a half-life of about 1 to 3 hours at 100° C. For example, the amplification reaction components can comprise one or more hyperthermophile DNA polymerases from Archaea (e.g., hyperthermophile DNA polymerases from Thermococcus, or hyperthermophile DNA polymerases from Thermococcaceaen archaean). In some embodiments, amplification reaction components comprise one or more hyperthermophile DNA polymerases from Pyrococcus, Methanococcaceae, Methanococcus, or Thermus. In some embodiments, amplification reaction components comprise one or more hyperthermophile DNA polymerases from Thermus thermophiles.

In some embodiments, amplification reaction components comprise a hyperthermophile DNA polymerase or functional fragment thereof. A functional fragment generally retains one or more functions of a full-length polymerase, for example, the capability to polymerize DNA (e.g., in an amplification reaction). In some instances, a functional fragment performs a function (e.g., polymerization of DNA in an amplification reaction) at a level that is at least about 50%, at least about 75%, at least about 90%, at least about 95% the level of function for a full length polymerase. Levels of polymerase activity can be assessed, for example, using a detectable nucleic acid amplification method, such as a method described herein. In some embodiments, amplification reaction components comprise a hyperthermophile DNA polymerase comprising an amino acid sequence of SEQ ID NO: 1 or SEQ ID NO: 2, or a functional fragment of SEQ ID NO: 1 or SEQ ID NO: 2.

In some embodiments, amplification reaction components (e.g., one or more amplification reagents) comprise a polymerase comprising an amino acid sequence that is at least about 90% identical to a hyperthermophile polymerase or a functional fragment thereof. In some embodiments, amplification reaction components comprise a polymerase comprising an amino acid sequence that is at least about 90%, 95%, or 99% identical to the amino acid sequence of SEQ ID NO: 1 or SEQ ID NO: 2, or a functional fragment thereof.

The polymerase can possess reverse transcription capabilities. In such embodiments, the amplification reaction can amplify RNA targets, for example, in a single step without the use of a separate reverse transcriptase. Non-limiting examples of polymerases that possess reverse transcriptase capabilities include Bst (large fragment), 9° N DNA polymerase, 9° Nm™ DNA polymerase, Therminator™, Therminator™ II, and the like). Amplification reaction components can comprise one or more separate reverse transcriptases. In some embodiments, more than one polymerase is included in in an amplification reaction. For example, an amplification reaction may comprise a polymerase having reverse transcriptase activity and a second polymerase having no reverse transcriptase activity.

In some embodiments, one or more polymerases having exonuclease activity are used during amplification. In some embodiments, one or more polymerases having no or low exonuclease activity are used during amplification. In some embodiments, a polymerase having no or low exonuclease activity comprises one or more modifications (e.g., amino acid substitutions) that reduce or eliminate the exonuclease activity of the polymerase. For example, a modified polymerase having low exonuclease activity can have 10% or less exonuclease activity compared to an unmodified polymerase, for example less than about 10%, 9%, 8%, 7%, 6%, 5%, 4%, 3%, 2%, or 1% exonuclease activity compared to an unmodified polymerase. In some embodiments, a polymerase has no or low 5′ to 3′ exonuclease activity, and/or no or low 3′ to 5′ exonuclease activity. In some embodiments, a polymerase has no or low single strand dependent exonuclease activity, and/or no or low double strand dependent exonuclease activity. Nonlimiting examples of the modifications that can reduce or eliminate exonuclease activity for a polymerase include one or more amino acid substitutions at position 141 and/or 143 and/or 458 of SEQ ID NO: 1 (e.g., D141A, E143A, E143D and A485L), or at a position corresponding to position 141 and/or 143 and/or 458 of SEQ ID NO: 1.

### Detection and Quantification

The methods described herein can comprise detecting and/or quantifying nucleic acid amplification product(s). Amplification product(s) can be detected and/or quantified, for example, by any suitable detection and/or quantification method described herein. Non-limiting examples of detection and/or quantification methods include molecular beacon (e.g., real-time, endpoint), lateral flow, fluorescence resonance energy transfer (FRET), fluorescence polarization (FP), surface capture, 5′ to 3′ exonuclease hydrolysis probes (e.g., TAQMAN), intercalating/binding dyes, absorbance methods (e.g., colorimetric, turbidity), electrophoresis (e.g., gel electrophoresis, capillary electrophoresis), mass spectrometry, nucleic acid sequencing, digital amplification, a primer extension method (e.g., iPLEX™), Molecular Inversion Probe (MIP) technology from Affymetrix, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, AlphaScreen, SNPstream, genetic bit analysis (GBA), Multiplex minisequencing, SNaPshot, GOOD assay, Microarray miniseq, arrayed primer extension (APEX), Microarray primer extension, Tag arrays, Coded microspheres, Template-directed incorporation (TDI), colorimetric oligonucleotide ligation assay (OLA), sequence-coded OLA, microarray ligation, ligase chain reaction, padlock probes, invader assay, hybridization using at least one probe, hybridization using at least one fluorescently labeled probe, cloning and sequencing, the use of hybridization probes and quantitative real time polymerase chain reaction (QRT-PCR), nanopore sequencing, chips and combinations thereof. In some embodiments, detecting a nucleic acid amplification product comprises use of a real-time detection method (i.e., product is detected and/or continuously monitored during an amplification process). In some embodiments, detecting a nucleic acid amplification product comprises use of an endpoint detection method (i.e., product is detected after completing or stopping an amplification process). Nucleic acid detection methods may also employ the use of labeled nucleotides incorporated directly into a target sequence or into probes containing complementary sequences to a target. Such labels may be radioactive and/or fluorescent in nature and can be resolved in any of the manners discussed herein. In some embodiments, quantification of a nucleic acid amplification product may be achieved using one or more detection methods described below. In some embodiments, the detection method can be used in conjunction with a measurement of signal intensity, and/or generation of (or reference to) a standard curve and/or look-up table for quantification of a nucleic acid amplification product.

Detecting a nucleic acid amplification product can comprise use of molecular beacon technology. The term molecular beacon generally refers to a detectable molecule, where the detectable property of the molecule is detectable under certain conditions, thereby enabling the molecule to function as a specific and informative signal. Non-limiting examples of detectable properties include optical properties (e.g., fluorescence), electrical properties, magnetic properties, chemical properties and time or speed through an opening of known size. Molecular beacons for detecting nucleic acid molecules can be, for example, hair-pin shaped oligonucleotides containing a fluorophore on one end and a quenching dye on the opposite end. The loop of the hair-pin can contain a probe sequence that is complementary to a target sequence and the stem is formed by annealing of complementary arm sequences located on either side of the probe sequence. A fluorophore and a quenching molecule can be covalently linked at opposite ends of each arm. Under conditions that prevent the oligonucleotides from hybridizing to its complementary target or when the molecular beacon is free in solution, the fluorescent and quenching molecules are proximal to one another preventing FRET. When the molecular beacon encounters a target molecule (e.g., a nucleic acid amplification product), hybridization can occur, and the loop structure is converted to a stable more rigid conformation causing separation of the fluorophore and quencher molecules leading to fluorescence. Due to the specificity of the probe, the generation of fluorescence generally is exclusively due to the synthesis of the intended amplified product. In some instances, a molecular beacon probe sequence hybridizes to a sequence in an amplification product that is identical to or complementary to a sequence in a target nucleic acid. In some instances, a molecular beacon probe sequence hybridizes to a sequence in an amplification product that is not identical to or complementary to a sequence in a target nucleic acid (e.g., hybridizes to a sequence added to an amplification product by way of a tailed amplification primer or ligation). Molecular beacons are highly specific and can discern a single nucleotide polymorphism. Molecular beacons also can be synthesized with different colored fluorophores and different target sequences, enabling simultaneous detection of several products in the same reaction (e.g., in a multiplex reaction). For quantitative amplification processes, molecular beacons can specifically bind to the amplified target following each cycle of amplification, and because non-hybridized molecular beacons are dark, it is not necessary to isolate the probe-target hybrids to quantitatively determine the amount of amplified product. The resulting signal is proportional to the amount of amplified product. Detection using molecular beacons can be done in real time or as an end-point detection method.

Detecting a nucleic acid amplification product can comprise use of lateral flow. Use of lateral flow typically includes use of a lateral flow device including but not limited to dipstick assays and thin layer chromatographic plates with various appropriate coatings. Immobilized on the flow path are various binding reagents for the sample, binding partners or conjugates involving binding partners for the sample and signal producing systems.

Detecting a nucleic acid amplification product can comprise use of FRET which is an energy transfer mechanism between two chromophores: a donor and an acceptor molecule. Briefly, a donor fluorophore molecule is excited at a specific excitation wavelength. The subsequent emission from the donor molecule as it returns to its ground state may transfer excitation energy to the acceptor molecule through a long range dipole-dipole interaction. The emission intensity of the acceptor molecule can be monitored and is a function of the distance between the donor and the acceptor, the overlap of the donor emission spectrum and the acceptor absorption spectrum and the orientation of the donor emission dipole moment and the acceptor absorption dipole moment. FRET can be useful for quantifying molecular dynamics, for example, in DNA-DNA interactions as described for molecular beacons. For monitoring the production of a specific product, a probe can be labeled with a donor molecule on one end and an acceptor molecule on the other. Probe-target hybridization brings a change in the distance or orientation of the donor and acceptor and FRET change is observed.

Detecting a nucleic acid amplification product can comprise use of fluorescence polarization (FP). FP techniques are based on the principle that a fluorescently labeled compound when excited by linearly polarized light will emit fluorescence having a degree of polarization inversely related to its rate of rotation. Therefore, when a molecule such as a tracer-nucleic acid conjugate, for example, having a fluorescent label is excited with linearly polarized light, the emitted light remains highly polarized because the fluorophore is constrained from rotating between the time light is absorbed and emitted. When a free tracer compound (i.e., unbound to a nucleic acid) is excited by linearly polarized light, its rotation is much faster than the corresponding tracer-nucleic acid conjugate and the molecules are more randomly oriented, therefore, the emitted light is depolarized. Thus, fluorescence polarization provides a quantitative means for measuring the amount of tracer-nucleic acid conjugate produced in an amplification reaction.

Detecting a nucleic acid amplification product can comprise use of surface capture, accomplished for example by the immobilization of specific oligonucleotides to a surface producing a biosensor that is both highly sensitive and selective. Example surfaces that can be used for attaching the probe include gold and carbon. Detecting a nucleic acid amplification product can comprise use of 5′ to 3′ exonuclease hydrolysis probes (e.g., TAQMAN). TAQMAN probes, for example, are hydrolysis probes that can increase the specificity of a quantitative amplification method (e.g., quantitative PCR). The TAQMAN probe principle relies on 1) the 5′ to 3′ exonuclease activity of Taq polymerase to cleave a dual-labeled probe during hybridization to a complementary target sequence and 2) fluorophore-based detection. A resulting fluorescence signal permits quantitative measurements of the accumulation of amplification product during the exponential stages of amplification, and the TAQMAN probe can significantly increase the specificity of the detection.

Detecting a nucleic acid amplification product can comprise use of intercalating and/or binding dyes, including dyes that specifically stain nucleic acid (e.g., intercalating dyes exhibit enhanced fluorescence upon binding to DNA or RNA). Dyes can include DNA or RNA intercalating fluorophores, including but not limited to, SYTO® 82, acridine orange, ethidium bromide, Hoechst dyes, PicoGreen®, propidium iodide, SYBR® I (an asymmetrical cyanine dye), SYBR® II, TOTO (a thiaxole orange dimer) and YOYO (an oxazole yellow dimer). Detecting a nucleic acid amplification product can comprise use of absorbance methods (e.g., colorimetric, turbidity). In some embodiments, detection and/or quantitation of nucleic acid can be achieved by directly converting absorbance (e.g., UV absorbance measurements at 260 nm) to concentration. Direct measurement of nucleic acid can be converted to concentration using the Beer Lambert law which relates absorbance to concentration using the path length of the measurement and an extinction coefficient. Detecting a nucleic acid amplification product can comprise use of electrophoresis (e.g., gel electrophoresis, capillary electrophoresis) and/or use of mass spectrometry. Mass Spectrometry is an analytical technique that can be used to determine the structure and quantity of a nucleic acid and can be used to provide rapid analysis of complex mixtures. Following amplification, samples can be ionized, the resulting ions separated in electric and/or magnetic fields according to their mass-to-charge ratio, and a detector measures the mass-to-charge ratio of ions. Mass spectrometry methods include, for example, MALDI, MALDI-TOF, and electrospray. These methods may be combined with gas chromatography (GC/MS) and liquid chromatography (LC/MS). Mass spectrometry (e.g., matrix-assisted laser desorption/ionization mass spectrometry (MALDI MS)) can be high throughput due to high-speed signal acquisition and automated analysis off solid surfaces.

Detecting a nucleic acid amplification product can comprise use of nucleic acid sequencing. The entire sequence or a partial sequence of an amplification product can be determined, and the determined nucleotide sequence may be referred to as a read. For example, linear amplification products may be analyzed directly without further amplification (e.g., by using single-molecule sequencing methodology). In some embodiments, linear amplification products is subject to further amplification and then analyzed (e.g., using sequencing by ligation or pyrosequencing methodology). Non-limiting examples of sequencing methods include single-end sequencing, paired-end sequencing, reversible terminator-based sequencing, sequencing by ligation, pyrosequencing, sequencing by synthesis, single-molecule sequencing, multiplex sequencing, solid phase single nucleotide sequencing, and nanopore sequencing. Detecting a nucleic acid amplification product can comprise use of digital amplification (e.g., digital PCR). Systems for digital amplification and analysis of nucleic acids are available (e.g., Fluidigm® Corporation).

### Lysis Buffers

### Lytic Agents

As disclosed herein, the lytic agents can comprise a detergent. The detergent can comprise one or more of a cationic surfactant, an anionic surfactant, a non-ionic surfactant, and an amphoteric surfactant. The anionic surfactant can comprise NH₄⁺, K⁺, Na⁺, or Li⁺ as a counter ion. The cationic surfactant can comprise I⁻, Br⁻, or Cl⁻ as a counter ion.

The anionic surfactant can be selected from potassium laurate, triethanolamine stearate, ammonium lauryl sulfate, lithium dodecyl sulfate, sodium lauryl sulfate, sodium alkyl sulfate (C8-16), sodium dodecyl sulfate, alkyl polyoxyethylene sulfate, sodium alginate, dioctyl sodium sulfosuccinate, phosphatidyl glycerol, phosphatidyl inositol, phosphatidylserine, phosphatidic acid and salts thereof, glyceryl ester, sodium carboxymethylcellulose, bile acid and salts thereof, cholic acid, deoxycholic acid, glycocholic acid, taurocholic acid, glycodeoxycholic acid, alkyl sulfonate, aryl sulfonate, alkyl phosphate, alkyl sulfonate, stearic acid and salts thereof, calcium stearate, phosphate, sodium carboxymethyl cellulose, dioctyl sulfosuccinate, dialkyl ester of sodium sulfosuccinic acid, phospholipid and calcium carboxymethyl cellulose.

The cationic surfactant can be selected from quaternary ammonium compounds, benzalkonium chloride, cetyl trimethyl ammonium bromide, chitonic acid, lauryl dimethyl benzyl ammonium chloride, acyl carnitine hydrochloride, alkyl pyridinium halide, cetylpyridinium chloride, cationic lipids, polymethylmethacrylate trimethyl ammonium bromide, sulfonium compounds, polyvinylpyrrolidone-2-dimethylaminoethyl methacrylate dimethyl sulfate, hexadecyl trimethyl ammonium bromide, phosphonium compounds, quaternary ammonium compounds, benzyl-di(2-chloroethyl)ethyl ammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₈-dimethyl hydroxyethyl ammonium chloride, C₁₂₋₁₅-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecyldimethylbenzyl ammonium chloride monohydrate, dimethyl didecyl ammonium chloride, N-alkyl (C₁₂₋₁₄)dimethyl 1-naphthylmethyl ammonium chloride, trimethylammonium halide alkyl-trimethylammonium salts, dialkyl-dimethylammonium salts, lauryl trimethyl ammonium chloride, ethoxylated alkyamidoalkyldialkylammonium salts, ethoxylated trialkyl ammonium salts, dialkylbenzene dialkylammonium chloride, N-didecyldimethyl ammonium chloride, N-tetradecyldimethylbenzyl ammonium chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₈₋₁₆ trimethyl ammonium bromide, C₈₋₁₆ trimethyl ammonium chloride, C₁₅ trimethyl ammonium bromide, C₁₇trimethyl ammonium bromide, dodecylbenzyl triethyl ammonium chloride, polydiallyldimethylammonium chloride, dimethyl ammonium chloride, alkyldimethylammonium halogenide, tricetyl methyl ammonium chloride, decyltrimethylammonium bromide, dodecyltriethylammonium bromide, tetradecyltrimethylammonium bromide, methyl trioctylammonium chloride, POLYQUAT 10, tetrabutylammonium bromide, benzyl trimethylammonium bromide, choline ester, benzalkonium chloride, stearalkonium chloride, cetyl pyridinium bromide, cetyl pyridinium chloride, halide salts of quaternized polyoxyethylalkylamines, MIRAPOL Alkaquat, alkyl pyridinium salts, amine, amine salts, imide azolinium salts, protonated quaternary acrylamides, methylated quaternary polymers, cationic gua gum, benzalkonium chloride, dodecyl trimethyl ammonium bromide, triethanolamine, and poloxamine.

The non-ionic surfactant can be selected from polyoxyethylene fatty alcohol ether, polyoxyethylene sorbitan fatty acid ester, polyoxyethylene fatty acid ester, polyoxyethylene alkyl ether, polyoxyethylene castor oil derivatives, sorbitan ester, glyceryl ester, glycerol monostearate, polyethylene glycol, polypropylene glycol, polypropylene glycol ester, cetyl alcohol, cetostearyl alcohol, stearyl alcohol, aryl alkyl polyether alcohol, polyoxyethylene polyoxypropylene copolymers, poloxamer, poloxamine, methylcellulose, hydroxycellulose, hydroxymethylcellulose, hydroxyethylcellulose, hydroxypropylcellulose, hydroxypropylmethylcellulose, hydroxypropylmethylcellulose phthalate, noncrystalline cellulose, polysaccharides, starch, starch derivatives, hydroxyethyl starch, polyvinyl alcohol, triethanolamine stearate, amine oxide, dextran, glycerol, gum acacia, cholesterol, tragacanth, and polyvinylpyrrolidone. The non-ionic surfactant can be alkyl sulfates, alkyl sulfonates, fatty acid soaps, salts of hydrox-, hydroperoxy-, polyhydroxy-, epoxy-fatty acids, salts of mono- and polycarboxylic acids, prostanoic acid and prostaglandins, leukotrienes and lipoxines, alkyl phosphates, alkyl phosphonates, sodium-dialkyl sufosuccinate, n-alkyl ethoxylated sulfates, cholate and desoxycholate of bile salts, perfluorocarboxylic acids, fluoroacliphatic phosphonates, or fluoroaliphatic sulphates.

The lytic agents provided herein can be capable of acting as a denaturing agent. “Denaturing agent” or “denaturant,” as used herein, shall be given its ordinary meaning and include any compound or material which will cause a reversible unfolding of a protein. The strength of a denaturing agent or denaturant will be determined both by the properties and the concentration of the particular denaturing agent or denaturant. Suitable denaturing agents or denaturants include chaotropes, detergents, organic solvents, water miscible solvents, phospholipids, or a combination of two or more such agents. Suitable chaotropes include, but are not limited to, urea, guanidine, and sodium thiocyanate. Useful detergents may include, but are not limited to, strong detergents such as sodium dodecyl sulfate, or polyoxyethylene ethers (e.g. Tween or Triton detergents), Sarkosyl, mild non-ionic detergents (e.g., digitonin), mild cationic detergents (e.g., N->2,3-(Dioleyoxy)-propyl-N,N,N-trimethylammonium), mild ionic detergents (e.g. sodium cholate or sodium deoxycholate) or zwitterionic detergents including, but not limited to, sulfobetaines (Zwittergent), 3-(3-chlolamidopropyl)dimethylammonio-1-propane sulfate (CHAPS), and 3-(3-chlolamidopropyl)dimethylammonio-2-hydroxy-1-propane sulfonate (CHAPSO). Organic, water miscible solvents such as acetonitrile, lower alkanols (especially C₂-C₄ alkanols such as ethanol or isopropanol), or lower alkandiols (especially C₂-C₄ alkandiols such as ethylene-glycol) may be used as denaturants. Phospholipids can be naturally occurring phospholipids such as phosphatidylethanolamine, phosphatidylcholine, phosphatidylserine, and phosphatidylinositol or synthetic phospholipid derivatives or variants such as dihexanoylphosphatidylcholine or diheptanoylphosphatidylcholine.

Suitable surfactant levels can be from about 0.1% to about 25%, from about 0.25% to about 10%, or from about 0.5% to about 5% by weight of the total composition. In some embodiments, the surfactants are anionic surfactants, amphoteric surfactants, nonionic surfactants, zwitterionic surfactants, cationic surfactants, and mixtures thereof. In some embodiments, it can be advantageous to use anionic, amphoteric, nonionic and zwitterionic surfactants (and mixtures thereof).

Useful anionic surfactants herein include the water-soluble salts of alkyl sulphates and alkyl ether sulphates having from 10 to 18 carbon atoms in the alkyl radical and the water-soluble salts of sulphonated monoglycerides of fatty acids having from 10 to 18 carbon atoms. Sodium lauryl sulphate and sodium coconut monoglyceride sulphonates are examples of anionic surfactants of this type.

Suitable cationic surfactants can be broadly defined as derivatives of aliphatic quaternary ammonium compounds having one long alkyl chain containing from about 8 to 18 carbon atoms such as lauryl trimethylammonium chloride; cetyl pyridinium chloride; benzalkonium chloride; cetyl trimethylammonium bromide; di-isobutylphenoxyethyl-dimethylbenzylammonium chloride; coconut alkyltrimethyl-ammonium nitrite; cetyl pyridinium fluoride; etc. Certain cationic surfactants can also act as germicides in the compositions disclosed herein.

Suitable nonionic surfactants that can be used in the compositions, methods and kits of the present disclosure can be broadly defined as compounds produced by the condensation of alkylene oxide groups (hydrophilic in nature) with an organic hydrophobic compound which may be aliphatic and/or aromatic in nature. Examples of suitable nonionic surfactants include the poloxamers; sorbitan derivatives, such as sorbitan di-isostearate; ethylene oxide condensates of hydrogenated castor oil, such as PEG-30 hydrogenated castor oil; ethylene oxide condensates of aliphatic alcohols or alkyl phenols; products derived from the condensation of ethylene oxide with the reaction product of propylene oxide and ethylene diamine; long chain tertiary amine oxides; long chain tertiary phosphine oxides; long chain dialkyl sulphoxides and mixtures of such materials. These materials are useful for stabilizing foams without contributing to excess viscosity build for the consumer product composition.

Zwitterionic surfactants can be broadly described as derivatives of aliphatic quaternary ammonium, phosphonium, and sulphonium compounds, in which the aliphatic radicals can be straight chain or branched, and wherein one of the aliphatic substituents contains from about 8 to 18 carbon atoms and one contains an anionic water-solubilizing group, e.g., carboxy, sulphonate, sulphate, phosphate or phosphonate.

Exemplary anionic, single-chain surface active agents include alkyl sulfates, alkyl sulfonates, alkyl benzene sulfonates, and saturated or unsaturated fatty acids and their salts. Moieties comprising the polar head group in the cationic surfactant can include, for example, quaternary ammonium, pyridinium, sulfonium, and/or phosphonium groups. For example, the polar head group can include trimethylammonium. Exemplary cationic, single-chain surface active agents include alkyl trimethylammonium halides, alkyl trimethylammonium tosylates, and N-alkyl pyridinium halides.

Alkyl sulfates can include sodium octyl sulfate, sodium decyl sulfate, sodium dodecyl sulfate, and sodium tetra-decyl sulfate. Alkyl sulfonates can include sodium octyl sulfonate, sodium decyl sulfonate, and sodium dodecyl sulfonate. Alkyl benzene sulfonates can include sodium octyl benzene sulfonate, sodium decyl benzene sulfonate, and sodium dodecyl benzene sulfonate. Fatty acid salts can include sodium octanoate, sodium decanoate, sodium dodecanoate, and the sodium salt of oleic acid.

Alkyl trimethylammonium halides can include octyl trimethylammonium bromide, decyl trimethylammonium bromide, dodecyl trimethylammonium bromide, myristyl trimethylammonium bromide, and cetyl trimethylammonium bromide. Alkyl trimethylammonium tosylates can include octyl trimethylammonium tosylate, decyl trimethylammonium tosylate, dodecyl trimethylammonium tosylate, myristyl trimethylammonium tosylate, and cetyl trimethylammonium tosylate. For example, N-alkyl pyridinium halides can include decyl pyridinium chloride, dodecyl pyridinium chloride, cetyl pyridinium chloride, decyl pyridinium bromide, dodecyl pyridinium bromide, cetyl pyridinium bromide, decyl pyridinium iodide, dodecyl pyridinium iodide, cetyl pyridinium iodide.

The cationic surfactant can comprise at least one compound selected from dodecyltrimethylammonium bromide, tetradecyltrimethylammonium bromide, cetyltrimethylammonium bromide, cetyldimethylethylammonium bromide, (C1 to C30 alkyl)-trimethylammonium bromide, a (C1 to C30 alkyl)amine, a (C1 to C30 alkyl) imidazoline, ethoxylated amine, a quaternary compound, a quaternary ester, a (C1 to C30 alkyl)amine oxide, lauramine oxide, dicetyldimonium chloride, cetrimonium chloride, a primary polyethoxylated fatty amine salt, a secondary polyethoxylated fatty amine salt, a tertiary polyethoxylated fatty amine salt, a quaternary ammonium salt, a tetra(C1 to C30 alkyl)ammonium halide, a (C1 to C30 alkyl)amide-(C1 to C30-alkyl)ammonium halide, a tri(C1 to C30 alkyl)benzylammonium halide, a tri(C1 to C30 alkyl)hydroxy-(C1 to C30 alkyl)ammonium halide, a (C1 to C30 alkyl)pyridinium chloride, a (C1 to C30 alkyl)pyridinium bromide, and a amine oxide.

The anionic surfactant can comprise at least one compound selected from sodium dodecyl sulfate, a (C6 to C30 alkyl)benzene sulfonate, a C6 to C30 alpha olefin sulfonate, a paraffin sulfonate, a (C6 to C30 alkyl) ester sulfonate, a (C6 to C30 alkyl) sulfate, a (C6 to C30 alkyl alkoxy) sulfate, a (C6 to C30 alkyl) sulfonate, a (C6 to C30 alkyl alkoxy) carboxylate, a (C6 to C30 alkyl alkoxylated) sulfate, a mono(C1 to C30 alkyl)(ether) phosphate, a di(C6 to C30 alkyl)(ether) phosphate, a (C6 to C30 alkyl) sarcosinate, a sulfosuccinate, sodium bis(2-ethylhexyl) sulfosuccinate, ethoxylate 4-nonylphenyl ether glycolic acid, a (C1 to C30 alkyl) isethionate, taurate, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl phosphate, sodium tridecyl phosphate, sodium behenyl phosphate, sodium laureth-2 phosphate, sodium ceteth-3 phosphate, sodium trideceth-4 phosphate, sodium dilauryl phosphate, sodium ditridecyl phosphate, sodium ditrideceth-6 phosphate, sodium lauroyl sarcosinate, lauroyl sarcosine, cocoyl sarcosine, ammonium cocoyl sulfate, sodium cocoyl sulfate, sodium trideceth sulfate, sodium tridecyl sulfate, ammonium trideceth sulfate, ammonium tridecyl sulfate, sodium cocoyl isethionate, disodium laureth sulfosuccinate, sodium methyl oleoyl taurate, sodium laureth carboxylate, sodium trideceth carboxylate, sodium lauryl sulfate, potassium cocoyl sulfate, potassium lauryl sulfate, monoethanolamine cocoyl sulfate, sodium tridecyl benzene sulfonate, sodium dodecyl benzene sulfonate, and sodium dodecyl sulfate.

The non-ionic surfactant can comprise at least one compound selected from, a C6 to C18 alkyl alcohol, a (C6 to C18 alkyl) phenol, a (C6 to C18 alkyl) ethoxylate, a (C6 to C18 alkyl) phenol (C1 to C3 alkoxylate), a block oxy(C1 to C3 alkylene) condensate of a C6 to C18 alkyl phenol, an oxy(C₁ to C₃ alkylene) condensate of alkanol, an oxyethylene/oxypropylene block copolymer, an amine oxide, a phosphine oxide, an alkylamine oxide having 8 to 50 carbon atoms, a mono or di(C8 to C30) alkyl alkanolamide, a (C6 to C30 alkyl) polysaccharide, a sorbitan fatty acid ester, a polyoxyethylene sorbitan fatty acid ester, a polyoxyethylene sorbitol ester, a polyoxyethylene nonylphenyl ether, a polyoxyethylenic acid, a polyoxyethylene alcohol, a coco monoethanolamide, a coco diethanolamide, a coco diglycoside, a (C8 to C30 alkyl) polyglycoside, cocamidopropyl, lauramine oxide, polyoxyethylene (20) sorbitan monolaurate, an ethoxylated linear C8 to C30 alcohol, cetearyl alcohol, lanolin alcohol, stearic acid, glyceryl stearate, polyethylene glycol 100 stearate, 4-(1,1,3,3-tetramethylbutyl)phenyl polyethylene glycol, polyoxyethylene (10) cetyl ether, eicosaethylene glycol octadecyl ether, and HO(CH₂CH₂O)₂₀(CH₂CH(CH₃)O)₇₀(CH₂CH₂O)₂₀H.

### Reducing Agents

The lysis buffer and/or reagent composition (e.g., dried composition) can comprise one or more reducing agents. A “reducing agent” can be a compound or a group of compounds. As used herein, “reducing agent”, also known as “reductant,” “reducer,” or “reducing equivalent,” can refer to an element or compound that donates an electron to another species. In particular, a reducing agent is generally a compound that breaks disulfide bonds by reduction, thereby overcoming those tertiary protein folding and quaternary protein structures (oligomeric subunits) which are stabilized by disulfide bonds. Examples of a suitable reducing agent include, but are not limited to, 2-mercaptoethanol, DTT, TCEP, DTE, reduced glutathione, cysteamine, TBP, dithioerythriol, THPP, 2-mercaptoethylamin-HCl, DTBA, cysteine, cysteine-thioglycolate, salts of sulfurous acid, thioglycolic acid and HED. In some embodiments of the methods, compositions and kits provided herein, the lysis buffer and/or reagent composition (e.g., dried composition) does not comprise one or more reducing agents.

### Reagent Composition

The reagent compositions described herein (e.g., dried composition) can be provided in a “dry form,” or in a form not suspended in liquid medium. The “dry form” of the compositions can include dry powders, lyophilized compositions, spray-dried, or precipitated compositions. The “dry form” compositions can include one or more lyoprotectants, such as sugars and their corresponding sugar alcohols, such as sucrose, lactose, trehalose, dextran, erythritol, arabitol, xylitol, sorbitol, and mannitol; amino acids, such as arginine or histidine; lyotropic salts, such as magnesium sulfate; polyols, such as propylene glycol, glycerol, poly(ethylene glycol), or polypropylene glycol); and combinations thereof. Additional exemplary lyoprotectants include gelatin, dextrins, modified starch, and carboxymethyl cellulose. As used herein, the terms “lyophilization,” “lyophilized,” and “freeze-dried” refer to a process by which the material to be dried is first frozen and then the ice or frozen solvent is removed by sublimation in a vacuum environment. “Lyophilisate” refers to a lyphophilized substance.

The reagent composition (e.g., dried composition) can be frozen or lyophilized or spray dried. The reagent composition can be heat dried. The reagent composition can comprise one or more additives (e.g., an amino acid, a polymer, a sugar or sugar alcohol). The sugar or sugar alcohol can comprise sucrose, lactose, trehalose, dextran, erythritol, arabitol, xylitol, sorbitol, mannitol, or any combination thereof. The polymer can comprise polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof. Lyophilized reagents can include poly rA, EGTA, EDTA, Tween 80, and/or Tween 20.

The frozen or lyophilized or spray dried or heat dried composition or the aqueous composition for preparing the frozen or lyophilized or spray dried composition may comprise one or more of the following: (i) Non-aqueous solvents such as ethylene glycol, glycerol, dimethylsulphoxide, and dimethylformamide. (ii) Surfactants such as Tween 80, Brij 35, Brij 30, Lubrol-px, Triton X-10; Pluronic F127 (polyoxyethylene-polyoxypropylene copolymer) also known as poloxamer, poloxamine, and sodium dodecyl sulfate. (iii) Dissacharides such as trehalose, sucrose, lactose, and maltose. (iv) Polymers (which may have different MWs) such as polyethylene glycol, dextran, polyvinyl alcohol), hydroxypropyl methylcellulose, gelatin, polyvinylpyrrolidone, hydroxyethyl cellulose, Ficoll, and albumin. (v) Amino acids such as glycine, proline, 4-hydroxyproline, L-serine, glutamate, alanine, lysine, sarcosine, and gamma-aminobutyric acid.

As disclosed herein, the reagent composition (e.g., dried composition) can comprise one or more protectants and one or more amplification reagents.

### Protectants

Provided herein are methods and compositions comprising one or more protectants and/or one or more additives. The one or more additives can comprise Tween 20, Triton X-100, Tween 80, a non-ionic detergent (e.g., a non-ionic surfactant), or any combination thereof. The one or more protectants can comprise a cyclodextrin compound (e.g., a compound of formula I). Cyclodextrins (CD) can be employed for complexation with lytic agents (e.g., SDS). Cyclodextrins (CDs) can be cyclic oligosaccharides which resemble truncated cones with hydrophobic inner cavity and hydrophilic outer surface The most commonly used natural cyclodextrins include 6, 7, and 8 glucose units, named as α, β and γ-CD. Natural CDs have can have solubility. Chemical modified CDs such as hydroxypropyl derivatives improve solubility up to 50% in aqueous media. CAVASOL® is the trade name of WACKER's cyclodextrin derivatives, which covers a variety of α, β and γ-CD derivatives. β-CD can form a strong inclusion complex (more so than α-CD and β-CD) with sodium dodecyl sulfate (SDS) in a predominately 1:1 stoichiometry. The binding constant of β-CD to SDS can range from 2100 M⁻¹ to 2500 M⁻¹.

The protectants described herein (e.g., cyclodextrin compounds) can be employed with lysis buffers comprising lytic agents (e.g., SDS) for complexation, such as lysis buffers for Chlamydia trachomatis and/or Neisseria gonorrhoeae detection. SDS is one of the most commonly used reagents for Chlamydia cell lysis. SDS can solubilize the phospholipid and protein components of the cell membrane, leading to cell lysis and release of the cell contents. SDS can also binds to proteins fairly specifically which denatures proteins at high concentration range. SDS can have an inhibitory effect at a concentration greater than 0.01% (w/v) for PCR with Taq polymerase. Provided herein include composition in methods wherein CD-SDS complexation occurs in real-time assays for Chlamydia trachomatis and/or Neisseria gonorrhoeae detection. SDS can be used in lysis buffer for cell lysis/sample preparation. CDs can be incorporated in a dried (e.g., lyophilized, heat dried) pellet containing enzyme, and other assay components. After lysis step, a sample solution can be mixed with a dried (e.g., lyophilized, heat dried) pellet containing cyclodextrin to facilitate CD and SDS inclusion complexation. The methods and compositions provided herein can comprise Chlamydia cell lysis buffers containing SDS (e.g., RIPA buffer: 50 mM Tris (pH 7.5), 150 mM NaCl, 0.1% SDS, 0.5% sodium deoxycholate, 1% NP-40; 1% SDS buffer: 50 mM Tris (pH7.5), 150mMNaCl, 1% SDS). The one or more protectants can comprise (2-Hydroxypropyl)-p-cyclodextrin (Cat #332593-5G), which can comprise average Mw ˜1,380, and 0.6 molar substitution. The one or more protectants can comprise (2-Hydroxypropyl)-β-cyclodextrin (Cat #389145-5G), which can comprise average Mw ˜1,540, and 1.0 molar substitution dextrin. The one or more protectants can comprise (2-Hydroxypropyl)-γ-cyclodextrin (Cat #H125-5G-I), which can comprise a water solubility of 450 mg/mL. The one or more protectants can comprise α-Cyclodextrin (Cat #C4642-5G), which can comprise a water solubility of 50 mg/mL.

The molar ratio of the one or more protectants to the one or more amplification reagents (e.g., amplification reaction components) in the amplification reaction mixture can be between about 10:1 to about 1:10, e.g., about 2:1. The ratio can be, be about, be at least, or be at most, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values.

The one or more protectants can comprise a cyclodextrin compound, such as a cyclodextrin compound of formula (I):

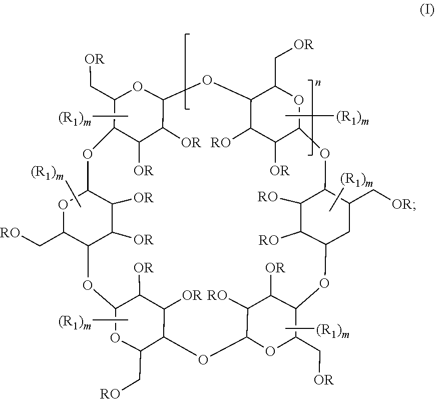

or a salt, ester, solvate or hydrate thereof, wherein,
each R is independently H, alkyl, alkenyl, alkynyl, cycloalkyl, heterocycloalkyl, aryl, or heteroaryl, each of which is optionally substituted; or —C(O)OR^{B}, —OC(O)R^{B}, —C(O)R^{B}, or —C(O)NR^{A}R^{B};
each R₁ is independently H, alkyl, alkenyl, alkynyl, cycloalkyl, aryl, heteroaryl, halogen, hydroxy, amino, —CN, —CF₃, —N₃, —NO₂, —OR^{B}, —SR^{B}, —SOR^{B} —SO₂R^{B}, —N(R^{B})S(O₂) —R^{B}, —N(R^{B}) S(O₂)NR^{A}R^{B}, —NR^{A}R^{B}, —C(O)OR^{B}, —OC(O)R^{B}, —C(O)R^{B}, —C(O)NR^{A}R^{B}, or —N(R^{B})C(O)R^{B}; each of which is optionally substituted;
each R^{A} is independently hydrogen, alkyl, alkenyl, alkynyl, cycloalkyl, heterocycloalkyl, aryl, or heteroaryl, each of which is optionally substituted;
each R^{B} is independently hydrogen, alkyl, alkenyl, alkynyl, cycloalkyl, heterocycloalkyl, aryl, or heteroaryl, each of which is optionally substituted;
n is 1,2,3,4,5,6,7,8,9, or 10;and
each m is independently 0, 1, 2, 3, 4, or 5.

In some embodiments, each R is independently H, optionally substituted alkyl, —C(O)OR^{B}, —OC(O)R^{B}, —C(O)R^{B}, or —C(O)NR^{A}R^{B}. In some embodiments, n is 1, 2, or 3. Each R can be independently H, methyl, ethyl, propyl, butyl, pentyl, hexyl, heptyl, or octyl; wherein each can be straight chain or branched.

The cyclodextrin compound can be 2-hydroxypropyl-α-cyclodextrin, 2-hydroxypropyl-β-cyclodextrin (2HPβCD), hydroxypropyl-β-cyclodextrin (HPβCD), methyl-β-cyclodextrin (MPCD), 2-Hydroxypropyl-γ-cyclodextrin, α-cyclodextrin, β-cyclodextrin, or γ-cyclodextrin, or a salt, ester, solvate or hydrate thereof.

The number of carbon atoms in a hydrocarbyl substituent can be indicated by the prefix “Cₓ-C_{y},” where x is the minimum and y is the maximum number of carbon atoms in the substituent. Likewise, a Cₓ chain means a hydrocarbyl chain containing x carbon atoms.

The prefix “halo” indicates that the substituent to which the prefix is attached is substituted with one or more independently selected halogen radicals. For example, “C₁-C₆haloalkyl” means a C₁-C₆alkyl substituent wherein at least one hydrogen radical is replaced with a halogen radical.

If a linking element in a depicted structure is “absent” or “a bond”, then the left element in the depicted structure is directly linked to the right element in the depicted structure. For example, if a chemical structure is depicted as X—(L)ₙ—Y wherein L is absent or n is 0, then the chemical structure is X—Y.

The term “alkyl” as used herein, refers to a saturated, straight- or branched-chain hydrocarbon radical. For example, “C₁-C₈ alkyl” contains from one to eight carbon atoms. Examples of alkyl radicals include, but are not limited to, methyl, ethyl, propyl, isopropyl, n-butyl, ieri-butyl, neopentyl, n-hexyl, heptyl, octyl radicals and the like.

The term “alkenyl” as used herein, denotes a straight- or branched-chain hydrocarbon radical containing one or more double bonds. For example, “C₂-C₈ alkenyl” contains from two to eight carbon atoms. Alkenyl groups include, but are not limited to, for example, ethenyl, propenyl, butenyl, 1-methyl-2-buten-1-yl, heptenyl, octenyl and the like.

The term “alkynyl” as used herein, denotes a straight- or branched-chain hydrocarbon radical containing one or more triple bonds. For example, “C₂-C₈ alkynyl” contains from two to eight carbon atoms. Representative alkynyl groups include, but are not limited to, for example, ethynyl, 1-propynyl, 1-butynyl, heptynyl, octynyl and the like.

The term “cycloalkyl” denotes a monovalent group derived from a monocyclic or polycyclic saturated carbocyclic ring compound. Examples of cycloalkyl include, but not limited to, cyclopropyl, cyclobutyl, cyclopentyl, cyclohexyl, bicyclo [2.2.1]heptyl, and bicyclo [2.2.2]octyl and the like. The terms “carbocycle” or “carbocyclic” or “carbocyclyl” refer to a saturated (e.g., “cycloalkyl”), partially saturated (e.g., “cycloalkenyl” or “cycloalkynyl”) or completely unsaturated (e.g., “aryl”) ring system containing zero heteroatom ring atom. A carbocyclyl may be, without limitation, a single ring, or two or more fused rings, or bridged or spiro rings. A carbocyclyl may contain, for example from 3 to 10 ring members (i.e., C₃-C₁₀carbocyclyl, such as C₃-C₁₀cycloalkyl). A substituted carbocyclyl may have either cis or trans geometry. Representative examples of carbocyclyl groups include, but are not limited to, cyclopropyl, cyclobutyl, cyclopentyl, cyclohexyl, cycloheptyl, cyclooctyl, cyclopentenyl, cyclopentadienyl, cyclohexadienyl, adamantyl, decahydro-naphthalenyl, octahydro-indenyl, cyclohexenyl, phenyl, naphthyl, fluorenyl, indanyl, 1,2,3,4-tetrahydro-naphthyl, indenyl, isoindenyl, bicyclodecanyl, anthracenyl, phenanthrene, benzonaphthenyl (also known as “phenalenyl”), decalinyl, and norpinanyl and the like. A carbocyclyl group can be attached to the parent molecular moiety through any substitutable carbon atom of the group.

The term “aryl” refers to an aromatic carbocyclyl containing from 6 to 14 carbon ring atoms. Non-limiting examples of aryls include phenyl, naphthalenyl, anthracenyl, and indenyl and the like. An aryl group can be connected to the parent molecular moiety through any substitutable carbon atom of the group.

The term “heteroaryl” means an aromatic heterocyclyl typically containing from 5 to 18 ring atoms, wherein at least one ring atom is a heteroatom. A heteroaryl may be a single ring, or two or more fused rings. Non-limiting examples of five-membered heteroaryls include imidazolyl; furanyl; thiophenyl (or thienyl or thiofuranyl); pyrazolyl; oxazolyl; isoxazolyl; thiazolyl; 1,2,3-, 1,2,4-, 1,2,5-, and 1,3,4-oxadiazolyl; and isothiazolyl. Non-limiting examples of six-membered heteroaryls include pyridinyl; pyrazinyl; pyrimidinyl; pyridazinyl; and 1,3,5-, 1,2,4-, and 1,2,3-triazinyl. Non-limiting examples of 6/5-membered fused ring heteroaryls include benzothiofuranyl, isobenzothiofuranyl, benzisoxazolyl, benzoxazolyl, purinyl, and anthranilyl. Non-limiting examples of 6/6-membered fused ring heteroaryls include quinolinyl; isoquinolinyl; and benzoxazinyl (including cinnolinyl and quinazolinyl).

The term “heterocycloalkyl” refers to a non-aromatic 3—, 4-, 5-, 6- or γ-membered ring or a bi- or tri-cyclic group fused system, where at least one of the ring atoms is a heteroatom, and where (i) each 5-membered ring has 0 to 1 double bonds and each 6-membered ring has 0 to 2 double bonds, (ii) the nitrogen and sulfur heteroatoms may optionally be oxidized, (iii) the nitrogen heteroatom may optionally be quaternized, and (iv) any of the above rings may be fused to a benzene ring. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl and the like.

The terms “heterocyclic” or “heterocycle” or “heterocyclyl” refer to a saturated (e.g., “heterocycloalkyl”), partially unsaturated (e.g., “heterocycloalkenyl” or “heterocycloalkynyl”) or completely unsaturated (e.g., “heteroaryl”) ring system, where at least one of the ring atoms is a heteroatom (i.e., nitrogen, oxygen or sulfur), with the remaining ring atoms being independently selected from carbon, nitrogen, oxygen and sulfur. A heterocyclyl group can be linked to the parent molecular moiety via any substitutable carbon or nitrogen atom in the group, provided that a stable molecule results. A heterocyclyl may be, without limitation, a single ring. Non-limiting examples of single-ring heterocyclyls include furanyl, dihydrofuranyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiodiazolyl, oxathiazolyl, oxadiazoly, pyranyl, dihydropyranyl, pyridinyl, piperidinyl, pyridazinyl, pyrimidinyl, pyrazinyl, piperazinyl, triazinyl, isoxazinyl, oxazolidinyl, isoxazolidinyl, oxathiazinyl, oxadiazinyl, morpholinyl, azepinyl, oxepinyl, thiepinyl, or diazepinyl. A heterocyclyl may also include, without limitation, two or more rings fused together, for example, naphthyridinyl, thiazolpyrimidinyl, thienopyrimidinyl, pyrimidopyrimidinyl, or pyridopyrimidinyl. A heterocyclyl may comprise one or more sulfur atoms as ring members; and in some cases, the sulfur atom(s) is oxidized to SO or SO₂. The nitrogen heteroatom(s) in a heterocyclyl may or may not be quatemized, and may or may not be oxidized to N-oxide. In addition, the nitrogen heteroatom(s) may or may not be N-protected.

The terms “optionally substituted”, “optionally substituted alkyl,” “optionally substituted “optionally substituted alkenyl,” “optionally substituted alkynyl”, “optionally substituted carbocyclic,” “optionally substituted aryl”, “optionally substituted heteroaryl,” “optionally substituted heterocyclic,” and any other optionally substituted group as used herein, refer to groups that are substituted or unsubstituted by independent replacement of one, two, or three or more of the hydrogen atoms thereon with typical substituents including, but not limited to: -alkyl, -alkenyl, -alkynyl, -aryl, -arylalkyl, -heteroaryl, -heteroarylalkyl, -heterocycloalkyl, -cycloalkyl, -carbocyclic, -heterocyclic, —F, —CI, —Br, —I, —OH, protected hydroxy, alkoxy, oxo, thiooxo, —NO₂, —CN, CF₃, N₃, —NH₂, protected amino, —NH alkyl, —NH alkenyl, —NH alkynyl, —NH cycloalkyl, —NH-aryl, —NH— heteroaryl, —NH-heterocyclic, -dialkylamino, -diarylamino, -diheteroarylamino, —O-alkyl, —O— alkenyl, —O-alkynyl, —β-cycloalkyl, —O-aryl, —O-heteroaryl, —O-heterocyclic, —C(O)— alkyl, —C(O)— alkenyl, —C(O)-alkynyl, —C(O)-cycloalkyl, —C(O)-aryl, —C(O)-heteroaryl, —C(O)-heterocycloalkyl, —CONH₂, —CONH-alkyl, —CONH-alkenyl, —CONH-alkynyl, —CONH-cycloalkyl, —CONH-aryl, —CONH-heteroaryl, —CONH-heterocycloalkyl, —OCO₂-alkyl, —OCO₂-alkenyl, —OCO₂-alkynyl, —OCO₂-cycloalkyl, —OCO₂-aryl, —OCO₂-heteroaryl, —OCO₂-heterocycloalkyl, —OCONH₂, —OCONH— alkyl, —OCONH-alkenyl, —OCONH-alkynyl, —OCONH-cycloalkyl, —OCONH-aryl, —OCONH— heteroaryl, —OCONH-heterocycloalkyl, —NHC(O)-alkyl, —NHC(O)-alkenyl, —NHC(O)-alkynyl, -NHC(O)-cycloalkyl, —NHC(O)-aryl, —NHC(O)-heteroaryl, —NHC(O)-heterocycloalkyl, —NHCO₂—alkyl, —NHCO₂-alkenyl, —NHCO₂-alkynyl, —NHCO₂-cycloalkyl, —NHCO₂-aryl, —NHCO₂-heteroaryl, —NHCO₂-heterocycloalkyl, —NHC(O)NH₂, —NHC(O)NH-alkyl, —NHC(O)NH-alkenyl, —NHC(O)NH— alkenyl, —NHC(O)NH-cycloalkyl, —NHC(O)NH-aryl, —NHC(O)NH-heteroaryl, —NHC(O)NH— heterocycloalkyl, NHC(S)NH₂, —NHC(S)NH-alkyl, —NHC(S)NH-alkenyl, —NHC(S)NH-alkynyl, -NHC(S)NH-cycloalkyl, —NHC(S)NH-aryl, —NHC(S)NH-heteroaryl, —NHC(S)NH-heterocycloalkyl, —NHC(NH)NH₂, —NHC(NH)NH-alkyl, —NHC(NH)NH-alkenyl, —NHC(NH)NH-alkenyl, -NHC(NH)NH-cycloalkyl, —NHC(NH)NH-aryl, —NHC(NH)NH-heteroaryl, —NHC(NH)NH— heterocycloalkyl, —NHC(NH)-alkyl, —NHC(NH)-alkenyl, —NHC(NH)-alkenyl, —NHC(NH)— cycloalkyl, —NHC(NH)-aryl, —NHC(NH)-heteroaryl, —NHC(NH)-heterocycloalkyl, —C(NH)NH— alkyl, —C(NH)NH-alkenyl, —C(NH)NH-alkynyl, —C(NH)NH-cycloalkyl, —C(NH)NH-aryl, —C(NH)NH-heteroaryl, —C(NH)NH-heterocycloalkyl, —S(O)— alkyl, —S(O)-alkenyl, —S(O)-alkynyl, —S(O)-cycloalkyl, —S(O)-aryl, —S(O)-heteroaryl, —S(O)-heterocycloalkyl, —SO₂NH₂, —SO₂NH-alkyl, —SO₂NH-alkenyl, —SO₂NH-alkynyl, —SO₂NH-cycloalkyl, —SO₂NH-aryl, —SO₂NH-heteroaryl, —SO₂NH-heterocycloalkyl, —NHSO₂-alkyl, —NHSO₂-alkenyl, —NHSO₂-alkynyl, —NHSO₂-cycloalkyl, —NHSO₂-aryl, —NHSO₂-heteroaryl, —NHSO₂-heterocycloalkyl, —CH₂NH₂, —CH₂SO₂CH₃, polyalkoxyalkyl, polyalkoxy, -methoxymethoxy, -methoxyethoxy, —SH, —S-alkyl, —S-alkenyl, —S—alkynyl, —S-cycloalkyl, —S-aryl, —S-heteroaryl, —S-heterocycloalkyl, or methylthiomethyl.

It is understood that the aryls, heteroaryls, carbocycles, heterocycles, alkyls, and the like can be further substituted.

The terms “halo” and “halogen,” as used herein, refer to an atom selected from fluorine, chlorine, bromine and iodine.

The term “leaving group,” or “LG”, as used herein, refers to any group that leaves in the course of a chemical reaction involving the group and includes but is not limited to halogen, brosylate, mesylate, tosylate, triflate, p-nitrobenzoate, phosphonate groups, for example.

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “hydroxy protecting group,” as used herein, refers to a labile chemical moiety which is known in the art to protect a hydroxy group against undesired reactions during synthetic procedures. After said synthetic procedure(s) the hydroxy protecting group as described herein may be selectively removed. Non-limiting examples of hydroxy protecting groups include benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl(trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, and the like. In some embodiments, hydroxy protecting groups are acetyl (Ac or —C(O)CH₃), benzoyl (Bz or —C(O)C₆H₅), and trimethylsilyl (TMS or —Si(CH₃)₃).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Examples of amino protecting groups include, but are not limited to, t-butoxycarbonyl, 9-fluorenylmethoxycarbonyl, benzyloxycarbonyl, and the like.

The term “protected amino,” as used herein, refers to an amino group protected with an amino protecting group as defined above.

The term “alkylamino” refers to a group having the structure —N(RₐR_{b}), where Rₐ and R_{b} are independent H or alkyl.

Examples of acceptable salts include, but are not limited to, nontoxic acid addition salts, or salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, or magnesium salts, and the like. Further acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “ester” can refer to esters of the compounds formed by the process disclosed herein which hydrolyze in vivo and include those that break down readily in the human body to leave the parent compound or a salt thereof. Suitable ester groups include, for example, those derived from acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present disclosure is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration unless the text so states; thus a carbon-carbon double bond depicted arbitrarily herein as trans may be cis, trans, or a mixture of the two in any proportion.

### Kits

Disclosed herein include kits for detecting a target nucleic acid sequence in a sample. In some embodiments, the kit comprises: (a) a lysis buffer comprising one or more lytic agents, wherein the one or more lytic agents are capable of lysing biological entities to release sample nucleic acids comprised therein, wherein the sample nucleic acids are suspected of comprising a target nucleic acid sequence; and (b) a dried composition comprising one or more protectants and one or more amplification reagents, wherein the one or more amplification reagents comprise one or more components for amplifying a target nucleic acid sequence under isothermal amplification conditions. In some embodiments, said components comprise: (i) a first primer and a second primer, wherein the first primer is capable of hybridizing to a sequence of a first strand of the target nucleic acid sequence, and the second primer is capable of hybridizing to a sequence of a second strand of the target nucleic acid sequence; and (ii) an enzyme having a hyperthermophile polymerase activity capable of generating a nucleic acid amplification product. In some embodiments, the lysis buffer and/or reagent composition comprises one or more reducing agents. The one or more reducing agents can comprise one or more of 2-mercaptoethanol, DTT, TCEP, DTE, reduced glutathione, cysteamine, TBP, dithioerythriol, THPP, 2-mercaptoethylamin-HCl, DTBA, cysteine, cysteine-thioglycolate, salts of sulfurous acid, thioglycolic acid and HED.

The kit can comprise: at least one component providing real-time detection activity for a nucleic acid amplification product. The real-time detection activity can be provided by a molecular beacon. The reagent composition (e.g., dried composition) can comprise a reverse transcriptase and/or a reverse transcription primer.

In some embodiments, the molar ratio of the one or more protectants to the one or more amplification reagents is between about 10:1 to about 1:10 (e.g., about 2:1). In some embodiments, the one or more additives comprise Tween 20, Triton X-100, Tween 80, a non-ionic detergent (e.g., a non-ionic surfactant), or any combination thereof. In some embodiments, the one or more protectants comprises a cyclodextrin compound of formula (I). In some embodiments, the one or more lytic reagents comprise about 0.001% (w/v) to about 1.0% (w/v) (e.g., about 0.2% (w/v)) of the treated sample. In some embodiments, the one or more lytic agents comprise a detergent. The detergent can comprise one or more of a cationic surfactant, an anionic surfactant, a non-ionic surfactant, and an amphoteric surfactant. In some embodiments, it can be advantageous that the one or more protectants are capable of sequestering the one or more lytic agents, thereby preventing the denaturing of the one or more amplification reagents by the one or more lytic agents.

Kits can comprise, for example, one or more polymerases and one or more primers, and optionally one or more reverse transcriptases and/or reverse transcription primers, as described herein. Where one target is amplified, a pair of primers (forward and reverse) can be included in the kit. Where multiple target sequences are amplified, a plurality of primer pairs can be included in the kit. A kit can include a control polynucleotide, and where multiple target sequences are amplified, a plurality of control polynucleotides can be included in the kit.

The enzyme having a hyperthermophile polymerase activity can have an amino acid sequence that is at least about 90% or 95% identical to the amino acid sequence of SEQ ID NO: 1 or a functional fragment thereof. For example, the enzyme having a hyperthermophile polymerase activity can comprise the amino acid sequence of SEQ ID NO: 1.

The nucleic acid amplification product can be about 20 to 40 bases long. The nucleic acid amplification product can comprise: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

The biological entities can comprise one or more of prokaryotic cells, eukaryotic cells, viral particles, exosomes, protoplasts, and microvesicles. The biological entities can comprise a virus, a bacteria, a fungi, a protozoa, portions thereof, or any combination thereof. The target nucleic acid sequence can be a nucleic acid sequence of a virus, bacteria, fungi, or protozoa. The sample nucleic acids can be derived from a virus, bacteria, fungi, or protozoa.

Kits can also comprise one or more of the components in any number of separate vessels, chambers, containers, packets, tubes, vials, microtiter plates and the like, or the components can be combined in various combinations in such containers. Components of the kit can, for example, be present in one or more containers. In some embodiments, all of the components are provided in one container. In some embodiments, the enzymes (e.g., polymerase(s) and/or reverse transcriptase(s)) can be provided in a separate container from the primers. The components can, for example, be lyophilized, heat dried, freeze dried, or in a stable buffer. In some embodiments, polymerase(s) and/or reverse transcriptase(s) are in lyophilized form or heat dried form in a single container, and the primers are either lyophilized, heat dried, freeze dried, or in buffer, in a different container. In some embodiments, polymerase(s) and/or reverse transcriptase(s), and the primers are, in lyophilized form or heat dried form, in a single container.

Kits can further comprise, for example, dNTPs used in the reaction, or modified nucleotides, vessels, cuvettes or other containers used for the reaction, or a vial of water or buffer for re-hydrating lyophilized or heat-dried components. The buffer used can, for example, be appropriate for both polymerase and primer annealing activity.

Kits can also comprise instructions for performing one or more methods described herein and/or a description of one or more components described herein. Instructions and/or descriptions can be in printed form and can be included in a kit insert. A kit also can include a written description of an internet location that provides such instructions or descriptions.

Kits can further comprise reagents used for detection methods, for example, reagents used for FRET, lateral flow devices, dipsticks, fluorescent dye, colloidal gold particles, latex particles, a molecular beacon, or polystyrene beads.

### EXAMPLES

Some aspects of the embodiments discussed above are disclosed in further detail in the following examples, which are not in any way intended to limit the scope of the present disclosure.

### Example 1

Effect of anionic detergent SDS and HP-(CD on Neisseria Gonorrhea DNA Amplification

This example provides SDS complexation analyses of the methods and compositions provided herein. In particular, this example demonstrates the effect of anionic detergent SDS and HP-βCD on Neisseria gonorrhea DNA Amplification. Anionic detergent SDS at >0.01% is inhibitory to APA amplification of DNA, but the presence of the protective agent hydroxypropyl β-CD (βCD) allowed amplification to proceed despite≤0.2% SDS. The kinetics of hydroxypropyl β-CD sequestration of SDS were thus sufficiently rapid at a molar ratio of 2:1 (˜14 mM: −7 mM) to prevent 9° Nₘ™ DNA polymerase inactivation by SDS.

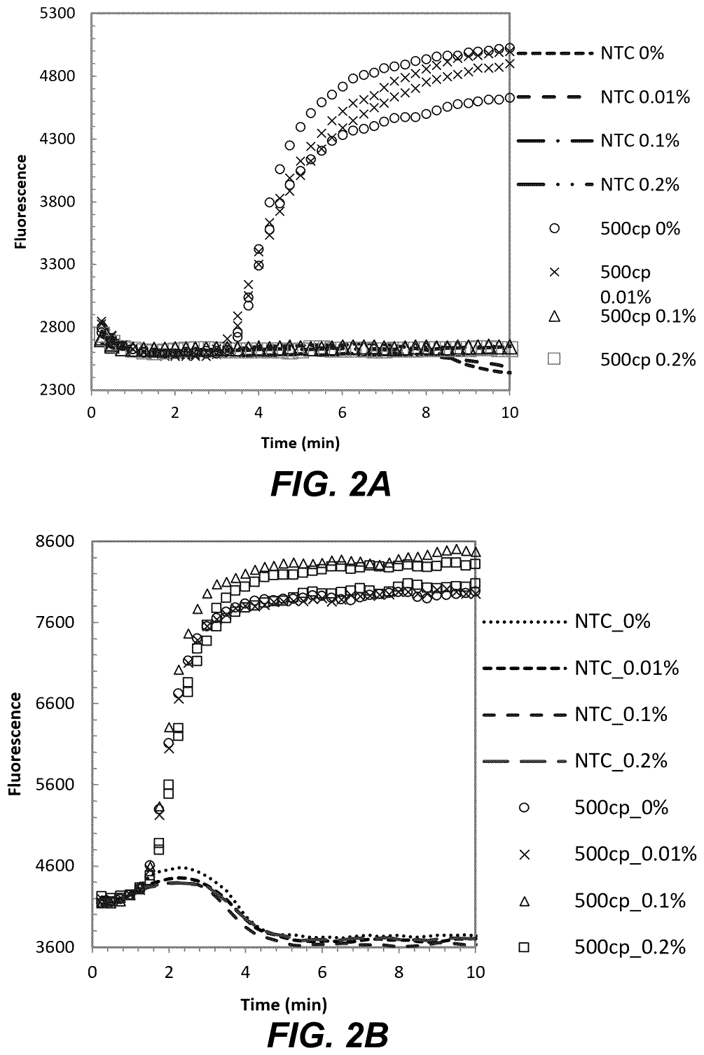

Standard APA reactions with or without 4% Sigma 389145 HPβCD containing “hot-start” CleanAmp™ deoxynucleotides (CAdNTP) and a sequence-specific molecular beacon detection probe were performed with lyophilized ((supplemented with dextran 40, Trehalose), FIG. 2B) or “wet” (no HPβCD, FIG. 2A) reagents including Tris pH 8.4, KCl, MgSO₄, and (NH₄)₂SO₄ Triton X-100, 9° Nₘ™ DNA polymerase, CAdNTP, primers and probe. For the lyophilized reactions, samples were prepared in 50 μl solution containing MgSO₄, and (NH₄)₂SO₄ with (shapes) or without (dashed) 500 copies of N. gonorrhea genomic DNA. After dried reagents were dissolved with the samples, various amounts of SDS were added, as indicated, and solutions were vortexed. For the wet reactions, 45 μl master mix containing all reaction components was added to 5 μl sample containing 1OX SDS (final concentration as indicated), with and without 500 copies of N. gonorrhea genomic DNA. Activation of CAdNTP by 2 minutes 90° C. was followed by APA at 68° C. for 10 minutes with detection of the NG amplification product by fluorescence collected every 15 seconds. The result showed inactivation by >0.01% SDS in the absence of βCD, but tolerance to at least 0.2% in its presence (FIGS. 2A-2B).

### Example 2

Effect of Cationic Detergent CTAB on Chlamydia trachomatis DNA Amplification

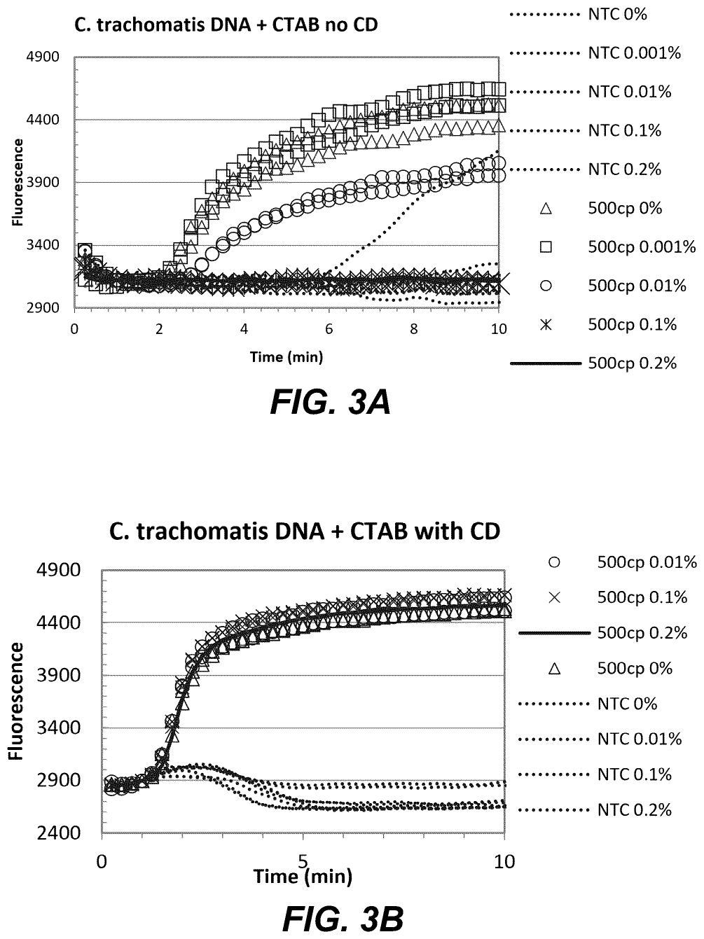

This example is similar to example 1 but provides a demonstration of the effect of cationic detergent CTAB on Chlamydia trachomatis DNA Amplification. APA reactions containing CAdNTP and sequence-specific molecular beacon detection probes were performed as above in lyophilized (with HP-βCD; FIG. 3B) or wet reactions (no HP-βCD; FIG. 3A) and various amounts of the cationic detergent CTAB, without (“NTC”) or with the addition of 500 copies of C. trachomatis total cell DNA. Activation of CAdNTP by heating 2 minutes at 90° C. was followed by APA at 68° C. with fluorescence collected every 15 seconds. The results showed complete inactivation by >0.01% CTAB in the absence of HP-βCD, but complete tolerance to at least 0.2% in its presence.

### Example 3

Lysis of Chlamydia trachomatis

This example demonstrates unexpected advantages of the methods and compositions provided herein over currently available nucleic acid detection methods and compositions. For example, even though the sample type of currently available methods can extend to human blood, which is easy to lyse, such methods may not provide efficient lysis nor amplification for hard-to-lyse pathogens.

Elementary body lysis (˜40/reaction) was probed in this example using an APA-based assay. Elementary bodies (Acrometrix) were added to either cold chlamydia transport media (SPD containing sucrose, phosphate buffer, and glutamic acid), TE (Tris-EDTA 10-1), or lysis buffer containing 0.4% SDS, pH 2.2. The samples in TE or SDS were heated 5 minutes at 95° C. (heat lysis) or 2 minutes at 78° C. (SDS lysis), respectively. Then 5 μl (containing 40 elementary bodies) was added to “wet” APA reaction mix as above (pH 7.6 to facilitate isothermal CAdNTP activation) containing 4% cyclodextrin HP-βCD for isothermal APA (68° C.) with fluorescence from a chlamydia-specific molecular beacon collected every 15 seconds.

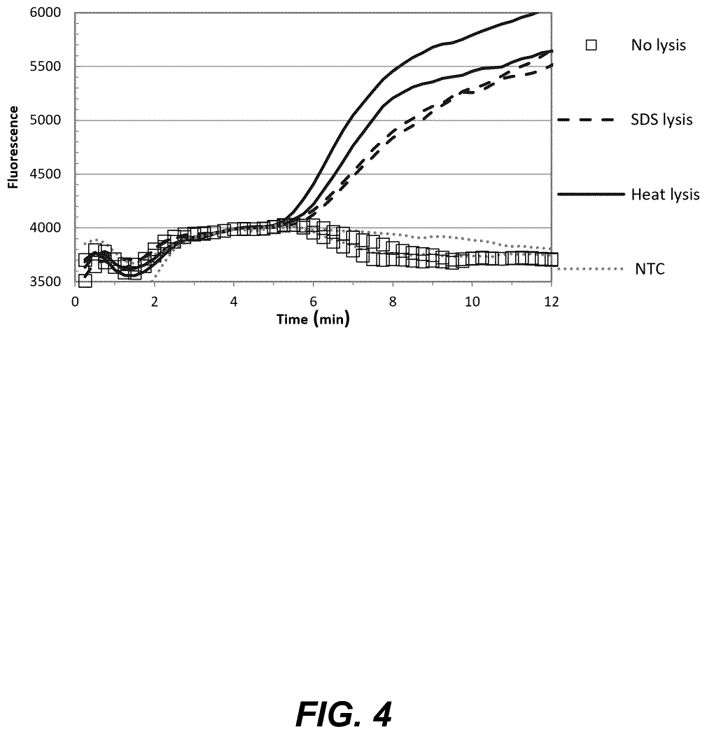

The SDS lysis (dashed) was as effective in liberating target DNA as the heat lysis (solid line). In the absence of lysis reagents, elementary bodies, which uniquely contain a rigid cross-linked protein cell wall, were not detectable (squares), like non-template controls (NTC dotted) (FIG. 4). Despite the presence of 0.4% ionic detergent to lyse the organisms, its persistence during APA, which would otherwise be inhibitory, had no impact on the 9° Nₘ™ DNA polymerase-mediated amplification due to the sequestration of the SDS by cyclodextrin.

### Example 4

Effect of hydroxypropyl-β-cyclodextrin (HPβCD) Protection on Flu B Virus Detection

This example demonstrates unexpected advantages of the methods and compositions provided herein over currently available nucleic acid detection methods and compositions. For example, unlike prior methods which do not disclose sensitivity of the assay to determine degree of protection, this example shows equivalent detection times +/− SDS with RNA target, with assay sensitivity determination. APA was performed with lyophilized reagents (including β-cyclodextrin, dNTPs, 35 units reverse transcriptase, KCl, EGTA (or EDTA), Tx-100, Tween 80, poly rA, Tris pH8.4, dextran 40k, 4% betaCD, trehalose, primers, probe, 9° N_{ml™)}

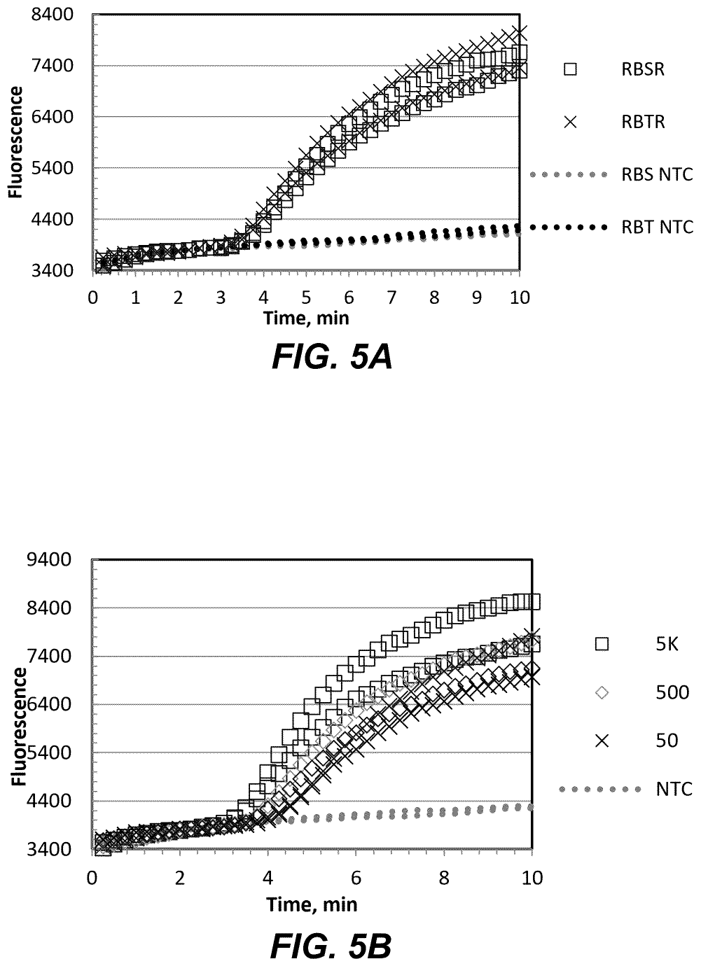

As shown in FIG. 5A, dried reagents preheated to 58° C. were dissolved without(NTC, dotted), or with the equivalent of 500 IU (infectious units) influenza B RNA by pipetting 50 ml lysis solution containing 0.2% SDS (RBSR) or 0.1% Triton X-100 (RBTR), MgSO₄, and (NH₄)₂SO₄ preheated to 58° C. for two minutes. After two minutes at 58° C. (to facilitate reverse transcription) the temperature was raised to 68° C. for APA and fluorescence detection.

As shown in FIG. 5B, APA demonstrated assay detection range (with SDS and virus) from 0 (NTC) to 5,000 IU of influenza B, showing 500 IU used in FIG. 5A was well within assay dynamic range, i.e., with sufficient assay sensitivity to imply virtually complete restoration of assay performance by HP-β-cyclodextrin in the presence of SDS under these conditions.

### Example 5

### Order-of-Addition Assay

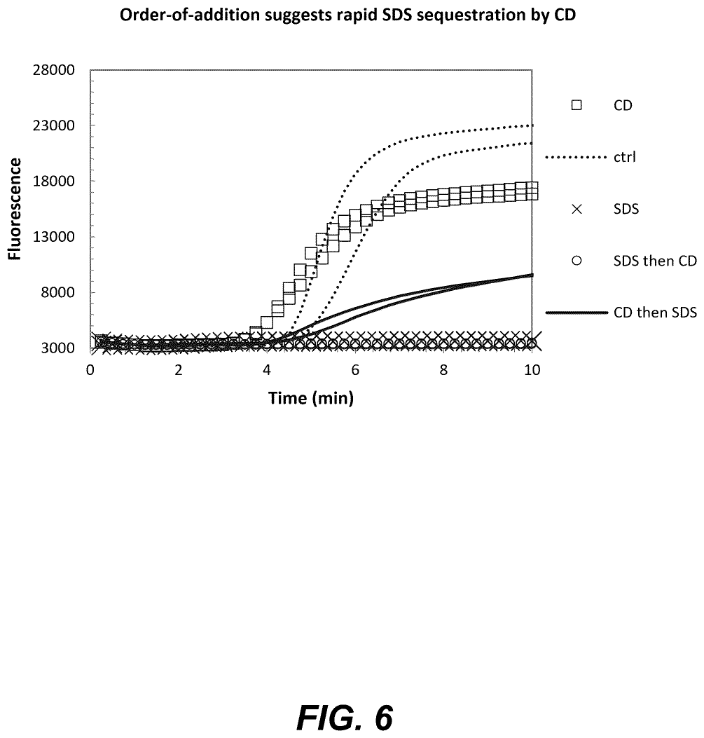

This example demonstrates unexpected advantages of the methods and compositions provided herein. For example, it was unexpectedly found that the kinetics of hydroxypropyl β-CD were sufficiently rapid at a molar ratio of 2:1 of hydroxypropyl β-CD and SDS, respectively. FIG. 6 depicts data related to an order-of-addition assay.

Order-of-addition assay: Detection time dependent on cyclodextrin addition prior to brief interaction of enzymes with SDS. Since SDS rapidly and irreversibly inactivated 9° Nₘ™ DNA polymerase, when the enzyme is instead simultaneously exposed to SDS and β-CD, the kinetics of complexation of the latter two substances must be very rapid, compared with the rate of interaction between SDS and the enzyme.

FIG. 6 depicts data related to detection of S. enterica DNA target (50,000 copies) by APA reactions in a wet “hot-start” format mediated by CleanAmp™ deoxynucleotides (CAdNTP) containing Tris pH7.6, KCl, (NH₄)₂SO₄, MgSO₄, Triton X-100, SYBRI, CAdNTP, 9° Nₘ™ DNA polymerase, with additions of 0.4% SDS (X), 4% hydroxypropyl β-CD (squares), SDS then immediately β-CD (circles), or β-CD then SDS (solid line) with mixing. Control (ctrl dotted line) was no additions. All samples included DNA target. Isothermal APA was at 68° C., collecting intercalating dye fluorescence every 15 seconds to monitor all exponential DNA amplification. Barrier to amplification from SDS (X, flat) was offset by prior addition of β-CD (solid line), restoring detection time, although with reduced plateau. Even a brief exposure to SDS prior to β-CD addition (circles, flat) was sufficient to inactivate the 9° Nₘ™ DNA polymerase enzyme, implying rapid kinetics of sequestration at this 2:1 molar ratio of β-CD:SDS. Negative controls for each condition without S. enterica DNA confirmed specificity of indicated amplification products (not shown).

### Example 6

### RNase A Inactivation Assays

Ionic detergents, such as sodium dodecyl sulfate (SDS) and/or cetyltrimethylammonium bromide (CTAB), can be employed as lytic agents in the methods and compositions provided herein, and can allow efficient lysis and also inhibit ribonucleases by denaturation, thus protecting the target RNA during the lysis step. However the subsequent direct, rapid molecular testing reaction, initiated by dissolution of a dried reaction mix directly by the lysed sample without separation or nucleic acid purification, can require rapid removal of the SDS (and/or CTAB), since it would also denature the enzymes reverse transcriptase and polymerase needed for the reaction. However, removal of some agents used to denature ribonuclease could thus enable its reactivation, i.e. restoration of its enzymatic activity. As shown herein, rapid removal of SDS (and/or CTAB) can be enabled by inclusion of a cyclodextrin in the dried reaction mix. This rapid removal may, in fact, also accelerate refolding of some denatured proteins. As demonstrated in this example, it was indeed shown that ribonuclease reactivation was very rapid in the presence of cyclodextrin, leaving the target RNA unprotected.

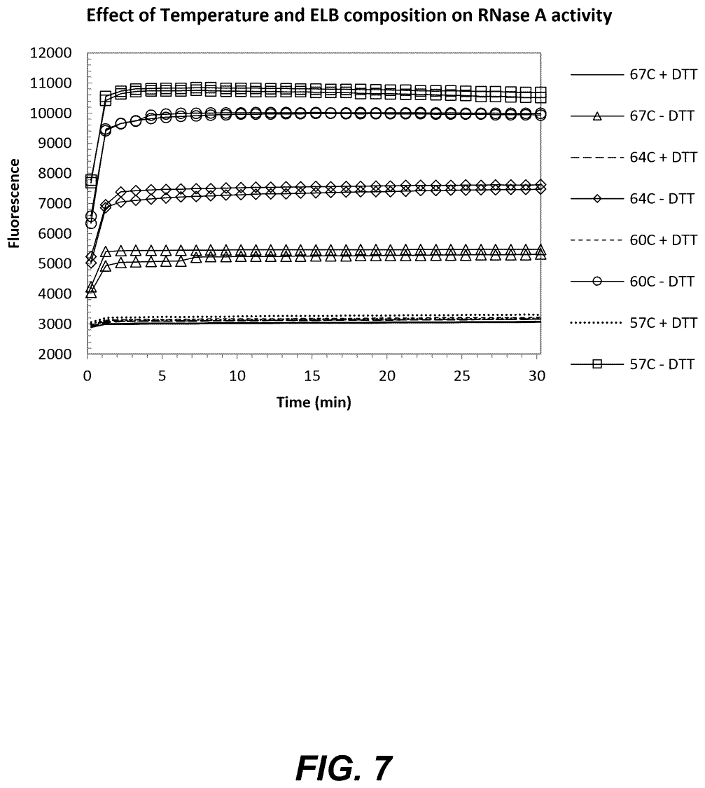

FIG. 7 depicts data related to the effect of DTT on the irreversibility of RNase A inactivation in the presence of CTAB at various temperatures. To simulate authentic reaction conditions, standard APA reaction reagents lacking 9° Nm™ DNA polymerase but containing 4% HP-βCD were prepared in a lyophilized format. The lyophilized reagents also contained the RNaseAlert® assay substrate, a synthetic quenched RNA probe which releases fluorescence in the presence of active RNase A. Reagents were preincubated 2 minutes and dissolved at the indicated temperatures with 50 ml solutions containing 20 ng RNase A (0.4 mg/ml) with (dotted lines) or without (shapes) 10 mM DTT which had also been preincubated at the indicated temperatures on a Bio-Rad CFx thermocycler with programmed temperature gradient. In all cases, immediate reversibility of presumed CTAB-mediated inactivation of RNase activity (without DTT, shapes) after CTAB sequestration by 4% HP-βCD was rendered irreversible by the inclusion of DTT, i.e., no restored activity was detected (flat lines).

### Example 7

### RNase A Inactivation Assays

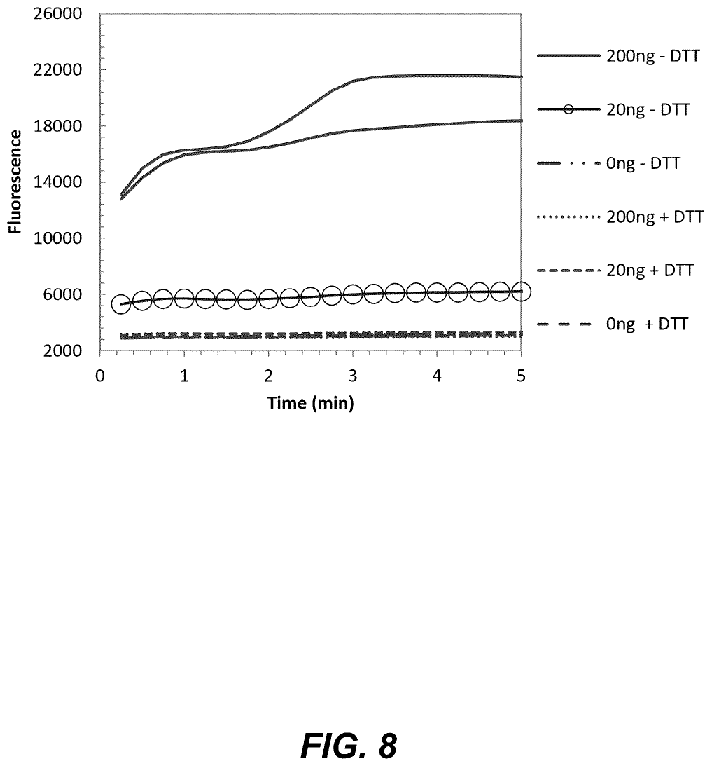

Currently available methods can only achieve irreversible ribonuclease inactivation at a maximum 200 ng/ml while the compositions and methods provided herein showed utility with 10 μg/ml to accommodate potentially larger amounts in patient samples. FIG. 8 depicts data related to effect of DTT and HP-βCD on RNase A inactivation. An RNase Alert assay was used to study the effect of DTT and 0.2% cationic detergent CTAB on RNase inactivation. RNase A was spiked into solutions containing 0.2% CTAB which were then mixed with lyophilized APA reagent pellets lacking enzymes but containing RNase Alert substrate and protective agent (4% HP-0 CD) after preincubation of all solutions at 68° C. followed by fluorescence data collection at 68° C. In the presence of 10 mM DTT (dotted and dashed lines; FIG. 8), RNase A was effectively irreversibly inactivated so that no activity was recovered upon sequestration of the detergent. With only detergent, however, activity was rapidly restored following sequestration (solid line 10 μg/ml, circles 1 μg/ml).

### Example 8

### Requirements for Irreversible RNase Inactivation

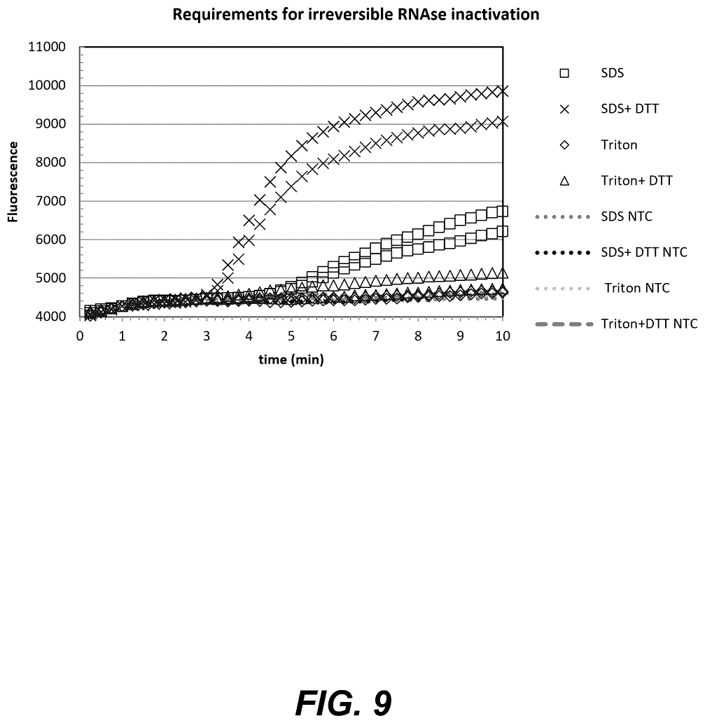

This example provides a demonstration that, unlike in some prior art, the combined action of DTT and SDS, but neither alone, enabled operationally irreversible inactivation of RNase A in a crude sample eluate prior to use of that undiluted sample to dissolve lyophilized APA reagents for APA without further treatment (FIG. 9). 500 ng RNase A (i.e., 10 μg/ml) was added to 50 ml APA running buffer with MgSO₄, and (NH₄)₂SO₄ containing the indicated additions (0.2% SDS, 10 mM DTT, 0.1% Triton X-100) without (dotted lines) or with (shapes) the equivalent of 2500 infectious units of purified Influenza B RNA and preheated 2 minutes at 58° C. (FIG. 9). The heated solution was then used to dissolve similarly preheated lyophilized APA reagents containing 9° Nm™ DNA polymerase, reverse transcriptase, and 4% βCD as used in Example 4. After a further two minutes at 58° C. (to facilitate reverse transcription with this set of primers), fluorescence was collected every 15 seconds after temperature adjustment to 68° C. to monitor APA. In this format, SDS alone (squares) did not prevent significant restoration of RNase A activity after SDS sequestration by 4% HP βCD (in the presence of SDS during the initial 2 minutes 58° C. incubation it was shown elsewhere there was no RNase activity). However, the combined action of DTT and SDS (X) allowed subsequent APA performance equivalent to positive control without RNase A (not shown). With Triton X-100, which does not impact RNase A activity, there was no RNA detection (diamond), with RNase A presumed active before and after dissolution of the APA reagents and initiation of reverse transcription. DTT and Triton, but no ionic detergent (triangle), did not protect RNA, again showing the inadequacy of DTT pretreatment to adequately protect RNA in this format. Assay calibration (not shown) was consistent with reduction in RNA detectability by >99% (FIG. 9). Importantly, this example shows that the prior art RNase inactivation (DTT without ionic detergent) is useless in protecting RNA for detection in this POC assay system using purified RNase A as a representative RNase. Moreover, ionic detergent alone was ineffective. While currently available methods can only achieve irreversible ribonuclease inactivation at a maximum 200 ng/ml, the compositions and methods provided herein show utility with 10 μg/ml to accommodate potentially larger amounts in patient samples. Thus, as shown here, simultaneous treatment with ionic detergent and reducing agent is required for successful detection under these conditions (e.g., with purified RNase A).

### Example 9

Protection of RNA from Nasal Swab Ribonucleases

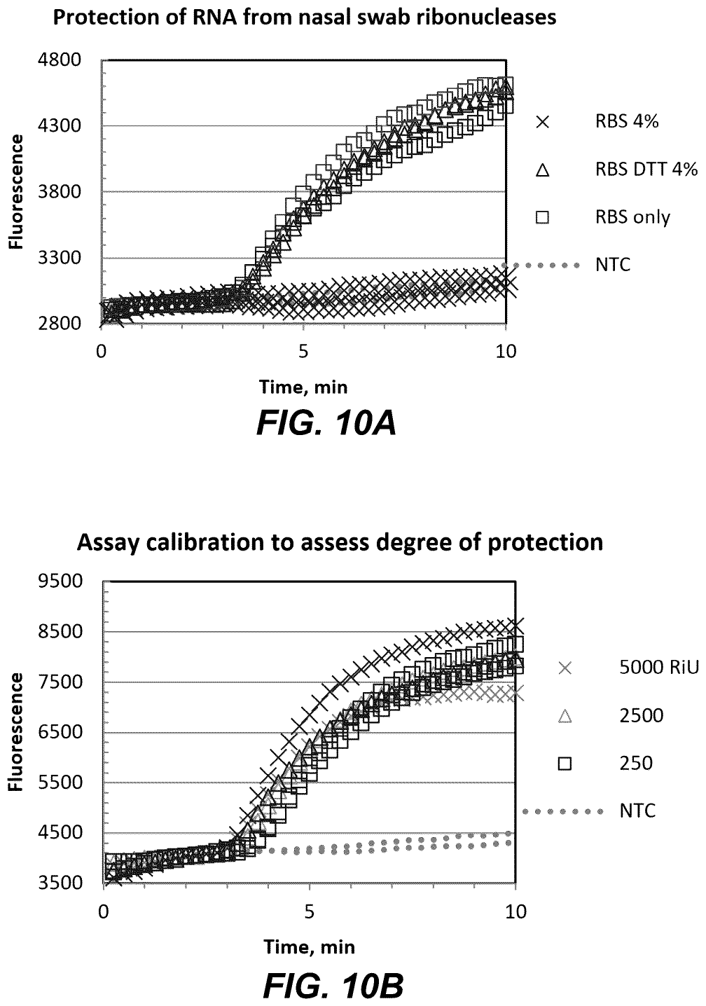

This example provides a demonstration of protection of RNA from nasal swab ribonucleases according to the methods and compositions provided herein. In FIG. 10A, nasal swab eluate (4% of swab contents, an amount likely encountered routinely during patient testing) was heated 2 minutes at 58° C. in a 50 ml solution containing 0.2% SDS (X) or 0.2% SDS+10 mM DTT (triangle). After adjustment to 4% βCD to sequester the SDS, influenza B RNA was added and incubation at 58° C. was continued. To monitor recovered ribonuclease activity during the second incubation (after SDS sequestration), 5 ml (containing the equivalent of 2500 infectious units of RNA) was tested by standard APA (incorporating standard RNase protection measures), i.e. lyophilized reagents and RBS DTT were preincubated at 58° C. and then reverse-transcription APA proceeded as in Example 4 Without DTT, there was no detection (X), while addition of DTT restored detection (triangle), implying recovered RNase activity without DTT vs. control (no nasal eluate, squares). NTC contained nasal eluate but no RNA. Assay calibration (FIG. 10B) showed that the recovered RNase activity was sufficient to eliminate >95% of the RNA detectability during the 2-minute incubation at 58° C. Thus, as shown here, simultaneous treatment with ionic detergent and reducing agent is required for successful detection under these conditions. Consistent with the data shown above, a potential risk to detectability of influenza RNA is addressed by the disclosed compositions and methods by showing SDS+ DTT-dependent irreversibility of inactivation of ribonucleases in human nasal cavity (e.g., those which would be encountered during customer use).

### Example 10

### RNase A Activity Assays

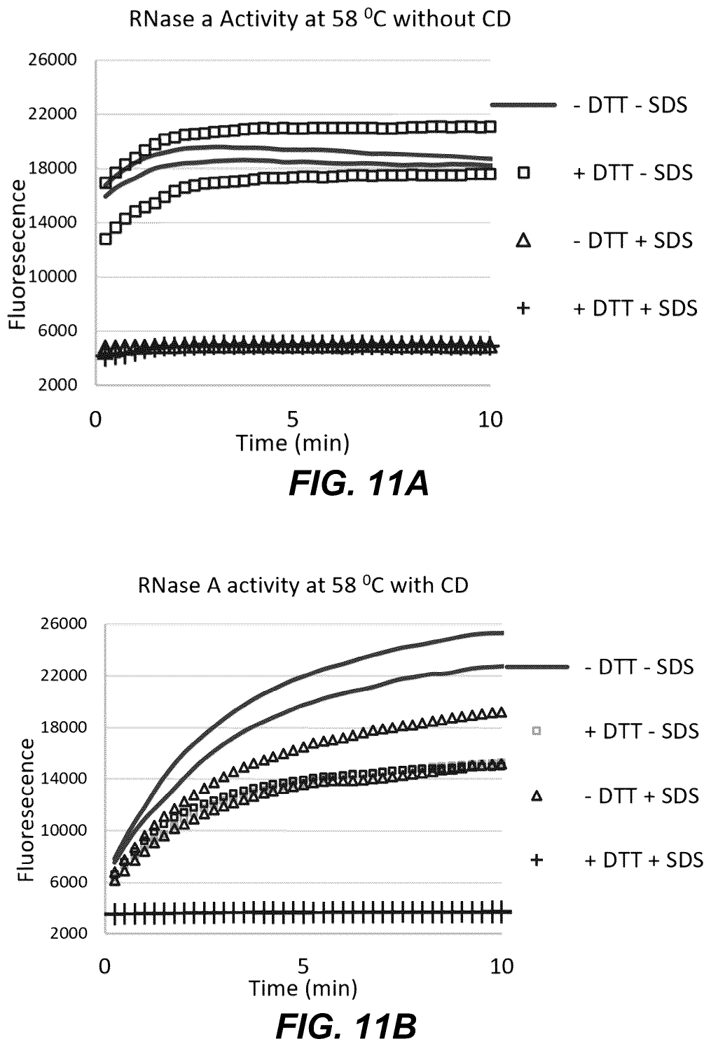

This example demonstrates unexpected advantages of the methods and compositions provided herein over currently available nucleic acid detection methods and compositions. 2 ng RNase A was incubated 2 minutes at 58° C. in 50 ml solutions containing either 10 mM DTT+0.2% SDS (+), 0.2% SDS (triangle), 10 mM DTT (square), or neither (solid line) and then the RNase Alert assay was used to study the recovery of any RNase activity upon sequestration of SDS after dissolving lyophilized APA reagents without enzymes but containing RNase Alert substrate and protective agent (4% βCD) with continued incubation and fluorescence data collection at 58° C. In the presence of RNase activity, the RNase alert indicator substrate releases detectable fluorescence. As shown in FIG. 11B, only the combination of DTT and SDS (+) prevented subsequent RNase recovery, consistent with its irreversible inactivation, while neither DTT (squares) or SDS (triangle) alone was adequate for preventing virtually full recovery of enzyme activity, nearly equivalent to control (solid line). FIG. 11A depicts recovery of RNase A activity after identical pretreatment without addition of βCD, in this case monitoring activity in 5 ml of the solution by transfer to an RNase alert “wet” reaction format. In this format, relevant conclusions are only drawn from the samples without SDS, since SDS would continue to inactivate RNAse A during the RNase alert assay, but the results did show that pretreatment with DTT alone (squares) also did not impact recovery of RNase A activity without subsequent βCD addition. Thus, as shown here, simultaneous treatment with ionic detergent and reducing agent is required for successful irreversible inactivation under the conditions described herein. Further, as demonstrated in this example, it was indeed shown that ribonuclease reactivation was very rapid in the presence of cyclodextrin, leaving the target RNA unprotected. Without being bound by any particular theory, this may be related to the impact of cyclodextrin and other APA additives on the refolding process. This example also provides further confirmation that the prior art and currently available methods (e.g, employing a reducing agent but no denaturant) are inadequate under these conditions, with an independent, commercially available RNase assay.

### Example 11

Influenza B assay

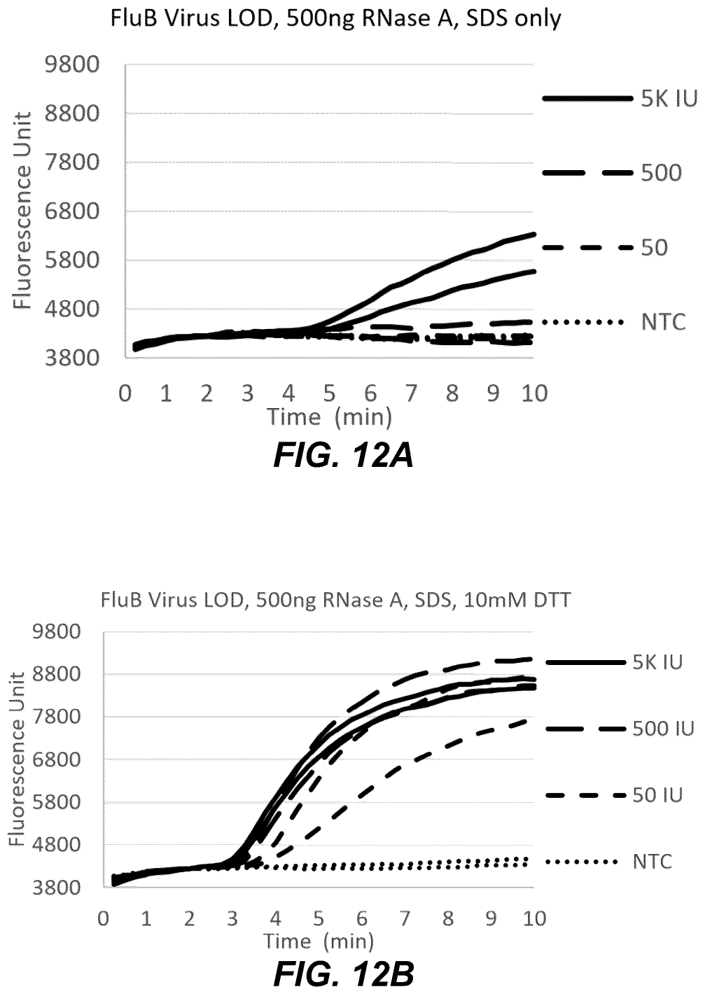

This example demonstrates unexpected advantages of the methods and compositions provided herein. FIGS. 12A-12B depict data related to RNase A inactivation in an influenza B assay like the one in Example 4, with DTT in ELB (Elution/Lysis Buffer). This example (FIG. 12A) demonstrates recovery of inhibitory levels of RNAse A (10 μg/ml) activity after heating 2 minutes 58° C. in ELB containing 0.2% SDS without (NTC dotted) or with (solid and dashed lines) various amounts of Influenza B virus. After incubation, lyophilized APA reagents co-incubated 2 minutes 58° C., containing β-CD (4% final) were dissolved with the heated solutions, and FAM fluorescence collected every 15 seconds to monitor APA. In FIG. 12B, the same conditions prevailed, except RNAse A was preincubated with the addition of 10 mM DTT to the 0.2% SDS, and this allowed performance equivalent to a no RNAse A control (not shown). The inclusion of DTT enables unimpeded operation of this POC influenza detection system in a mock sample containing very high level of purified RNase A (10 mg/ml). Moreover, while currently available methods can only achieve irreversible ribonuclease inactivation at a maximum 200 ng/ml, the compositions and methods provided herein show utility with 10 μg/ml to accommodate potentially larger amounts in patient samples.

### Example 12

### Rapid Irreversibility of Nasal Ribonuclease Inactivation

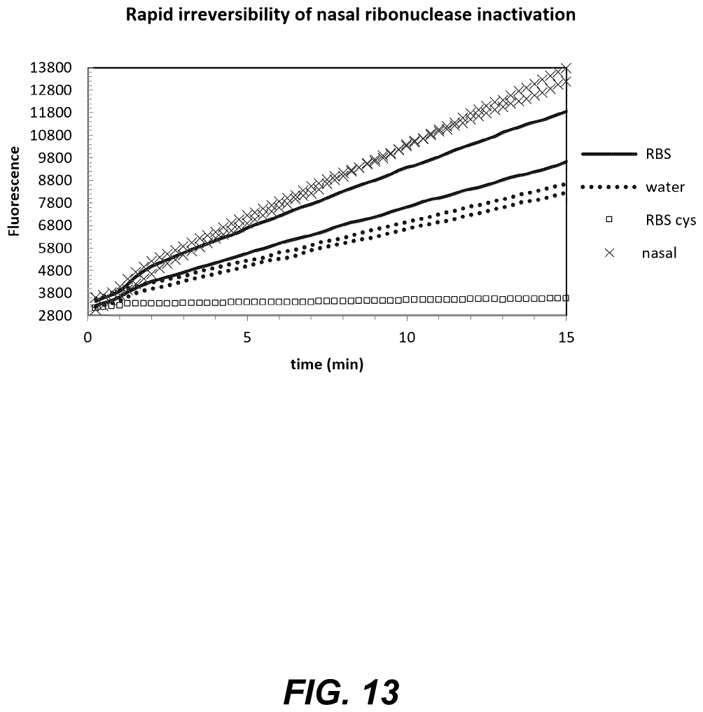

This example demonstrates unexpected advantages of the methods and compositions provided herein over currently available nucleic acid detection methods and compositions. FIG. 13 depicts data related to the rapid irreversibility of nasal ribonuclease inactivation. 5 μl concentrated nasal swab eluate in TE, an amount equivalent to 2% on a typical nasal swab, was mixed with 45 μl of either lysis buffer containing 0.2% SDS (RBS, solid line), RBS containing 40 mM cysteine (squares), or water (dotted line) all preheated to 60° C. 5 μl was immediately diluted into 45 μl 4% β-cyclodextrin to sequester detergent. The recovered RNase activity was determined with an RNase alert assay; as control for the assay, an equivalent amount of nasal swab eluate was directly added without prior treatment (X). The result was consistent with the idea that RNase inactivation mediated by the combined action of SDS and cysteine as reducing agent was operationally immediately irreversible (squares). In the presence of only SDS (solid line), RNase inactivation was virtually completely reversible after SDS sequestration by the cyclodextrin, i.e., indistinguishable from controls (dotted line or X). Unexpectedly, the effectively irreversible inactivation of ribonucleases thus observed during the practice of this invention was virtually instantaneous (monitored by directly testing ribonuclease activity), unlike prior art methods based upon heat. Also shown in this example is that the inactivation properties described for RNase A are also attributable to ribonucleases naturally occurring in nasal exudates.

### Example 13

### Direct Lysis for Isothermal Flu B Virus Detection in the Presence and Absence of Clinical Matrix

This example provides a demonstration of equivalent detection with and without inclusion of a nasal swab sample utilizing the methods disclosed herein.

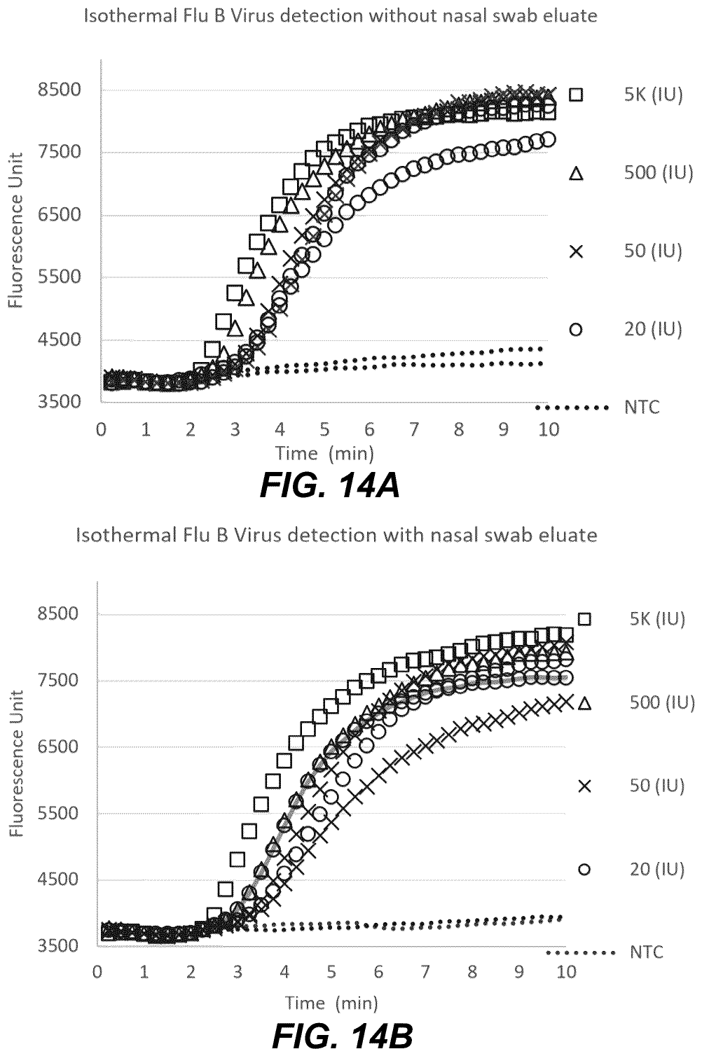

Live influenza B virus was lysed 2 minutes at 68° C. in 50 l μsolution containing 0.2% SDS, which was then used to dissolve lyophilized APA reagents containing HP-βCD at the same temperature, for isothermal reverse-transcription and APA as above with reverse transcriptase; 9dN+ Poly rA+ BSA without (FIG. 14A) and with (FIG. 14B) an amount of nasal swab eluate representing 4% of the amount on a typical nasal swab sample (containing ribonuclease activity) from a presumed negative individual included during lysis. Detection of amplified influenza B was with a fluorescein-labeled molecular beacon, with little or no difference with or without nasal “matrix” ribonucleases (FIGS. 14A-14B).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for detecting a target nucleic acid sequence in a sample, comprising:
(a) contacting a sample comprising biological entities with a lysis buffer to generate a treated sample, wherein the lysis buffer comprises one or more lytic agents capable of lysing the biological entities to release sample nucleic acids comprised therein, and wherein the sample nucleic acids are suspected of comprising a target nucleic acid sequence;
(b) contacting a reagent composition with the treated sample to generate an amplification reaction mixture, wherein the reagent composition comprises one or more protectants and one or more amplification reagents;
(c) amplifying the target nucleic acid sequence in the amplification reaction mixture, thereby generating a nucleic acid amplification product; and
(d) detecting the nucleic acid amplification product, wherein the detecting is performed in less than about 20 minutes from the time the reagent composition is contacted with the treated sample.

2. The method of claim 1, wherein the lysis buffer and/or reagent composition comprises one or more reducing agents.

3. (canceled)

4. (canceled)

5. (canceled)

6. The method of claim 1, wherein the one or more amplification reagents comprise a reverse transcriptase and/or an enzyme having a hyperthermophile polymerase activity.

7. (canceled)

8. (canceled)

9. The method of claim 1, wherein the reagent composition is lyophilized, heat-dried, and/or comprises one or more additives.

10. (canceled)

11. The method of claim 1, wherein the one or more protectants comprises a cyclodextrin compound of formula (I): 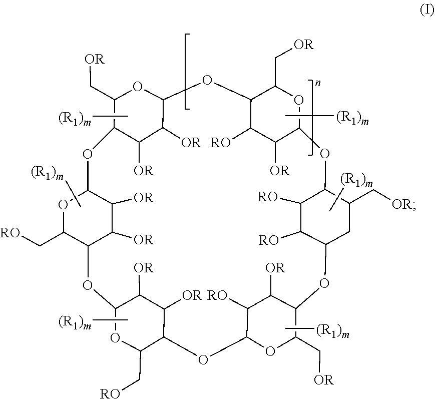
or a salt, ester, solvate or hydrate thereof, wherein,
each R is independently H, alkyl, alkenyl, alkynyl, cycloalkyl, heterocycloalkyl, aryl, or heteroaryl, each of which is optionally substituted; or —C(O)OR^{B}, —OC(O)R^{B}, —C(O)R^{B}, or —C(O)NR^{A}R^{B};
each R₁ is independently H, alkyl, alkenyl, alkynyl, cycloalkyl, aryl, heteroaryl, halogen, hydroxy, amino, —CN, —CF₃, —N₃, —NO₂, —OR^{B}, —SR^{B}, —SOR^{B}, —SO₂R^{B}, —N(R^{B})S(O₂) —R^{B}, —N(R^{B}) S(O₂)NR^{A}R^{B}, —NR^{A}R^{B}, —C(O)OR^{B}, —OC(O)R^{B}, —C(O)R^{B}, —C(O)NR^{A}R^{B}, or —N(R^{B})C(O)R^{B}; each of which is optionally substituted;
each R^{A} is independently hydrogen, alkyl, alkenyl, alkynyl, cycloalkyl, heterocycloalkyl, aryl, or heteroaryl, each of which is optionally substituted;
each R^{B} is independently hydrogen, alkyl, alkenyl, alkynyl, cycloalkyl, heterocycloalkyl, aryl, or heteroaryl, each of which is optionally substituted;
n is 1, 2, 3, 4, 5, 6, 7, 8, 9, or 10; and
each m is independently 0, 1, 2, 3, 4, or 5.

12. (canceled)

13. (canceled)

14. The method of claim 1, wherein the one or more lytic reagents comprise:
about 0.001% (w/v) to about 1.0 (w/v) of the treated sample.

15. (canceled)

16. The method of claim 1, wherein amplifying the target nucleic acid sequence comprises:
amplifying a target nucleic acid sequence comprising a first strand and a second strand complementary to each other in an isothermal amplification condition, wherein the amplifying comprises contacting a nucleic acid comprising the target nucleic acid sequence with:
i) a first primer and a second primer, wherein the first primer is capable of hybridizing to a sequence of the first strand of the target nucleic acid sequence, and the second primer is capable of hybridizing to a sequence of the second strand of the target nucleic acid sequence; and
ii) an enzyme having a hyperthermophile polymerase activity, thereby generating a nucleic acid amplification product, wherein the nucleic acid amplification product comprises:
(1) the sequence of the first primer, and the reverse complement thereof,
(2) the sequence of the second primer, and the reverse complement thereof, and
(3) a spacer sequence flanked by (1) the sequence of the first primer and the reverse complement thereof and (2) the sequence of the second primer and the reverse complement thereof, wherein the spacer sequence is 1 to 10 bases long.

17. (canceled)

18. (canceled)

19. (canceled)

20. (canceled)

21. The method of claim 1, wherein amplifying the target nucleic acid sequence comprises:
(c1) contacting sample ribonucleic acids with a reverse transcriptase and/or a reverse transcription primer to generate a cDNA;
(c2) contacting the cDNA with an enzyme having a hyperthermophile polymerase activity to generate a double-stranded DNA (dsDNA), wherein the dsDNA comprises a target nucleic acid sequence, and wherein the target nucleic acid sequence comprises a first strand and a second strand complementary to each other;
(c3) amplifying the target nucleic acid sequence under an isothermal amplification condition, wherein the amplifying comprises contacting the dsDNA with:
(i) a first primer and a second primer, wherein the first primer is capable of hybridizing to a sequence of the first strand of the target nucleic acid sequence, and the second primer is capable of hybridizing to a sequence of the second strand of the target nucleic acid sequence; and
(ii) the enzyme having a hyperthermophile polymerase activity, thereby generating a nucleic acid amplification product, wherein the nucleic acid amplification product comprises:
(1) the sequence of the first primer, and the reverse complement thereof,
(2) the sequence of the second primer, and the reverse complement thereof, and
(3) a spacer sequence flanked by (1) the sequence of the first primer and the reverse complement thereof and (2) the sequence of the second primer and the reverse complement thereof, wherein the spacer sequence is 1 to 10 bases long.

22. (canceled)

23. (canceled)

24. The method of claim 1, wherein the enzyme having a hyperthermophile polymerase activity has an amino acid sequence that is at least about 90% identical to the amino acid sequence of SEQ ID NO: 1 or a functional fragment thereof.

25. The method of claim 1, wherein amplifying the target nucleic acid sequence is performed at a constant temperature of about 55° C. to about 75° C., or at a constant temperature of about 65° C.

26. The method of claim 21, wherein:
the first primer, the second primer, and/or the reverse transcription primer is about 8 to 17 bases long;
the nucleic acid amplification product is about 20 to 40 bases long; and/or
the spacer sequence comprises a portion of the target nucleic acid sequence.

27. The method of claim 1, further comprising contacting the nucleic acid amplification product with a signal-generating oligonucleotide capable of hybridizing to the amplification product, wherein the signal-generating oligonucleotide comprises a fluorophore, a quencher, or both.

28. (canceled)

29. (canceled)

30. (canceled)

31. (canceled)

32. (canceled)

33. (canceled)

34. The method of claim 2, wherein the sample comprises a plurality of nucleases, wherein the nucleases comprise ribonucleases and/or deoxyribonucleases, and wherein:
the one or more lytic agents are capable of denaturing the ribonucleases to generate denatured ribonucleases, wherein the one or more reducing agents are capable of reducing the ribonucleases to generate reduced ribonucleases, and wherein the one or more reducing agents and the one or more lytic agents are capable of generating reduced-denatured ribonucleases; and/or
the one or more lytic agents are capable of denaturing the deoxyribonucleases to generate denatured deoxyribonucleases, wherein the one or more reducing agents are capable of reducing the deoxyribonucleases to generate reduced deoxyribonucleases, and wherein the one or more reducing agents and the one or more lytic agents are capable of generating reduced-denatured deoxyribonucleases.

35. The method of claim 34, wherein:
denatured ribonucleases and/or reduced-denatured ribonucleases are capable of renaturing upon passage of time and/or upon reduced physical interaction with the one or more lytic agents, thereby generating renatured ribonucleases; and/or
denatured deoxyribonucleases and/or reduced-denatured deoxyribonucleases are capable of renaturing upon passage of time and/or upon reduced physical interaction with the one or more lytic agents, thereby generating renatured deoxyribonucleases.

36. The method of claim 34, wherein denatured deoxyribonucleases, reduced deoxyribonucleases, reduced-denatured deoxyribonucleases, denatured ribonucleases, reduced ribonucleases, and/or reduced-denatured ribonucleases are enzymatically inactive, and wherein renatured ribonucleases and/or renatured deoxyribonucleases are enzymatically active.

37. The method of claim 34, wherein:
reduced-denatured ribonucleases renature at least about 1.1-fold more slowly than denatured ribonucleases; and/or
reduced-denatured deoxyribonucleases renature at least about 1.1-fold more slowly than denatured deoxyribonucleases.

38. The method of claim 1, wherein:
about 1.1-fold more sample ribonucleic acids are employed by the reverse transcriptase as template to generate cDNA as compared to a comparable method wherein the lysis buffer does not comprise one or more reducing agents; and/or
about 1.1-fold more sample deoxyribonucleic acids are employed by enzyme having a hyperthermophile polymerase activity as template to generate a nucleic acid amplification product to a comparable method wherein the lysis buffer does not comprise one or more reducing agents.

39. The method of claim 1, wherein the amplification reaction mixture comprises an at least 1.1-fold lower nuclease activity ten minutes after step (b) and/or step (c) as compared to a comparable method wherein the lysis buffer does not comprise one or more reducing agents.

40. (canceled)

41. (canceled)

42. (canceled)

43. (canceled)

44. (canceled)

45. (canceled)

46. (canceled)

47. A kit for detecting a target nucleic acid sequence in a sample, the kit comprising:
(a) a lysis buffer comprising one or more lytic agents capable of lysing biological entities to release sample nucleic acids comprised therein, wherein the sample nucleic acids are suspected of comprising a target nucleic acid sequence; and
(b) a reagent composition comprising one or more protectants and one or more amplification reagents comprising one or more components for amplifying the target nucleic acid sequence under isothermal amplification conditions, wherein said one or more components for amplifying comprise:
(i) a first primer and a second primer, wherein the first primer is capable of hybridizing to a sequence of a first strand of the target nucleic acid sequence, and the second primer is capable of hybridizing to a sequence of a second strand of the target nucleic acid sequence; and
(ii) an enzyme having a hyperthermophile polymerase activity capable of generating a nucleic acid amplification product.

48. The kit of claim 47, wherein the lysis buffer and/or reagent composition comprises one or more reducing agents.

49-60. (canceled)
